(19) United States
(12) Patent Application Publication
(10) Pub. No.: US 2024/0019435 A1
(43) Pub. Date: Jan. 18, 2024

(54) COMPOSITIONS, DEVICES AND METHODS COMPRISING MICROBE-TARGETING MOLECULES FOR DIAGNOSING AND TREATING INFECTIOUS DISEASE

(71) Applicant: MIRAKI INNOVATION THINK TANK LLC, Cambridge, MA (US)

(72) Inventors: Nisha Veronica VARMA, Brookline, MA (US); Keith CRAWFORD, Westwood, MA (US); Gregory T. MARTIN, Boston, MA (US); Goossen Jan Bernard BOER, Brookline, MA (US); Rainuka GUPTA, Newton, MA (US); Zhiqian ZHOU, Boston, MA (US); Christopher VELIS, Lexington, MA (US); Andrew D. CONNERTY, Boxford, MA (US)

(73) Assignee: MIRAKI INNOVATION THINK TANK LLC, Cambridge, MA (US)

(21) Appl. No.: 18/038,869

(22) PCT Filed: Sep. 24, 2021
(86) PCT No.: PCT/US21/51908

§ 371 Date: May 25, 2023

Related U.S. Application Data
(60) Provisional application No. 63/083,465, filed on Sep. 25, 2020.

(51) Int. Cl. G01N 33/569; G01N 33/543; A61M 1/36; C07K 14/47

(52) U.S. Cl. CPC G01N 33/569; G01N 33/54386; A61M 1/362; C07K 14/4726; G01N 2469/10

(57) ABSTRACT

The present invention provides biological molecules for use in detecting, identifying and/or removing microbes and microbial components; diagnostic, therapeutic and filtration devices comprising the biological molecules; and systems and methods for treating fluids using the biological molecules and devices of the invention.

### SEQUENCE LISTING

A sequence listing in electronic (ASCII text file) format is filed with this application and incorporated herein by reference. The name of the ASCII text file is “2021_1712A_ST25.txt”; the file was created on Sep. 24, 2021; the size of the file is 50 KB.

### FIELD OF THE INVENTION

The present disclosure relates to (i) biological molecules for use in detecting, identifying and/or removing microbes and microbial components in a sample or a target area, such as bodily fluids (e.g. blood and tissues), food, water, and environmental surfaces; (ii) diagnostic, therapeutic and filtration devices comprising the biological molecules, and to methods of using such devices in diagnosis, treatment and filtration; and (iii) systems and methods for treating fluids, including pathogen filtration from fluids, using the biological molecules and devices of the invention.

### BACKGROUND

There is a persistent need for affordable and highly sensitive tools that can be used to rapidly and accurately detect, identify and/or isolate microbes and microbial components in a sample. Such tools can be incorporated into assay devices for diagnostic applications, systems for removing pathogens, and therapeutic devices for treatment of subjects, to name only a few relevant applications.

For example, sepsis is a life-threatening condition that results from microbial infections (e.g., bacterial, viral, parasitic, or fungal) and the body's associated response causing damage to tissues. Sepsis is a major cause of death in American intensive care units. While microbes can directly damage tissues, resulting inflammatory responses can cause further damage and lead to septic shock and death. Early detection of infection and accurate identification of the infecting microbes are keys to successful treatment as different microbes are most susceptible to different treatments. Patients in septic shock should be treated as soon as possible to derive optimal benefit from antimicrobial therapies.

Rapid testing for, and identification of, microbes also has applications in food safety, environmental sampling, and other areas in addition to healthcare. However, means of capturing and rapidly and accurately identifying pathogens and other microbes directly from fluids (e.g., body fluids) are lacking. Furthermore, there are insufficient means for rapidly filtering fluids to selectively remove specific target microbes or classes of microbes where the ability to perform such filtration could be used to treat infections in a variety of fluids and to remove microbes and pathogens from water or other food or environmental fluids.

To meet some of these needs, a variety of biosensor products have been commercially developed and released. A specific example of a biosensor platform currently in use is the CANARY® biosensor technology of PathSensors, Inc. This platform, based on the work of Rider et al. (Science 2003, 301:213-215), enables reliable identification of specific airborne and liquid-based pathogens. The biological backbone of the CANARY® biosensor is comprised of a genetically-engineered B cell expressing an extracellularly bound, antigen-specific antibody that can bind its cognate antigen or pathogenic agent. In this system, when an antigen-containing sample interacts with the antibody on the extracellular surface of the biosensor, an intracellular signaling cascade is activated resulting in the release of Ca²⁺ within the B cells. In the CANARY® system, the B cells express aequorin, a Ca²⁺-sensitive photoprotein, which results in cell luminescence in the presence of elevated intracellular Ca²⁺ levels. Thus, the luminescence can be used to indicate antigen binding.

The CANARY® system can be used to efficiently identify a number of specific antigens, including those from bacteria, viruses, and toxins. However, expansion of the antigen test repertoire is complex and costly. Different antigen- or pathogen-specific biosensors must be constructed to recognized each and every selected antigen, which requires multiple steps including production of hybridoma cell lines, cloning of nucleic acid sequences encoding the antibodies, and expressing cloned antibodies as transmembrane proteins on the surface of a B cell line genetically engineered to luminesce upon binding of the cognate antigen (e.g., a pathogen) by the antibody. Thus, the diagnostic applications of the system remain limited. Furthermore, incorporation of this system into therapeutic devices would be difficult, if not impossible.

Thus, there remains a need for the development of universal and near-universal tools that can be adapted for use in multiple diagnostic and testing platforms across a broad range of environmental and pathogenic agents, and that can also be incorporated into devices and systems for removing such agents and into therapeutic devices for treating such agents. The present invention is directed to these and other important goals.

### BRIEF SUMMARY

The present invention is generally directed to (i) microbe-targeting molecules (MTMs) and engineered MTMs that have the shared characteristic of binding to one or more microbe-associated molecular patterns, (iii) diagnostic, therapeutic and filtration devices comprising the MTMs, and to methods of using such devices in diagnosis, treatment and filtration, and (iii) systems and methods for treating fluids, including pathogen filtration from fluids, using the MTMs and devices of the invention.

### MTMs

As summarized above, the invention is directed, in part, to “microbe-targeting molecules” (MTMs) and/or engineered MTMs that have the shared characteristic of binding to one or more microbe-associated molecular patterns (MAMPs), and to the use of such compositions in the treatment and/or prevention of infectious disease.

The important basis of the MTMs used in the compositions and devices of the invention, and the related methods, is that these constructs contact and bind microbes and microbial components based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

As used herein, “MTM” and “engineered MTM” refers to any of the molecules described herein (or described in patents or patent application incorporated by reference) that can bind to a microbe or microbe component. Unless the context indicates otherwise, the term “MTM” is used to describe all MTMs of the invention, both naturally-occurring and engineered forms of these constructs.

Given that the MTMs of the invention are defined based on their binding activity, it will be apparent that both naturally-occurring and engineered MTMs will comprise at least one microbe-binding domain, i.e. a domain that recognizes and binds to one or more MAMPs (including, at least two, at least three, at least four, at least five, or more) as described herein. A microbe-binding domain can be a naturally-occurring or a synthetic molecule. In some aspects, a microbe-binding domain can be a recombinant molecule. In addition to the microbe-binding domain, the MTMs of the invention will typically have one or more additional domains that may include, but are not limited to, an oligomerization domain, a signal domain, an anchor domain, a collagen-like domain, a fibrinogen-like domain, an immunoglobulin domain, and an immunoglobulin-like domain.

In a first embodiment, the present invention is directed to collectin-based MTMs. The collectin-based MTMs are either naturally-occurring collectin proteins or engineered MTM fusion proteins that comprise at least one collectin microbe-binding domain and at least one additional domain.

The naturally-occurring collectin protein may be any one of (i) mannose-binding lectin (MBL), (ii) surfactant protein A (SP-A), (iii) surfactant protein D (SP-D), (iv) collectin liver 1 (CL-L1), (v) collectin placenta 1 (CL-P1), (vi) conglutinin collectin of 43 kDa (CL-43), (vii) collectin of 46 kDa (CL-46), (viii) collectin kidney 1 (CL-K1), (ix) conglutinin, and (x) a sequence variant having at least 85% sequence identity to any one of (i)-(ix).

In one aspect of this embodiment, the collectin is (i) a naturally-occurring MBL, (ii) a truncated form of naturally-occurring MBL, (iii) an engineered form of MBL, or (iv) a sequence variant having at least 85% sequence identity to any one of (i), (ii) or (iii).

In a specific aspect of this embodiment, the collectin is a naturally-occurring MBL as set forth in SEQ ID NO:1 or a sequence variant having at least 85% sequence identity with SEQ ID NO:1 that retains the activity of the native protein.

In another specific aspect of this embodiment, the collectin is a truncated form of naturally-occurring MBL as set forth in any one of SEQ ID NOs:2-5 or a sequence variant having at least 85% sequence identity with any one of SEQ ID NOs:2-5 that retains the activity of the native protein.

The collectin microbe-binding domain of the collectin-based engineered MTMs comprises a carbohydrate recognition domain (CRD) of a collectin. The collectin may be any one of (i) mannose-binding lectin (MBL), (ii) surfactant protein A (SP-A), (iii) surfactant protein D (SP-D), (iv) collectin liver 1 (CL-L1), (v) collectin placenta 1 (CL-P1), (vi) conglutinin collectin of 43 kDa (CL-43), (vii) collectin of 46 kDa (CL-46), (viii) collectin kidney 1 (CL-K1), (ix) conglutinin, and (x) a sequence variant having at least 85% sequence identity to any one of (i)-(ix).

The at least one additional domain of the collectin-based engineered MTMs may be one or more of (xi) a collectin cysteine-rich domain, (xii) a collectin collagen-like domain, (xiii) a collectin coiled-coil neck domain, (xiv) a ficolin short N-terminal domain, (xv) a ficolin collagen-like domain, (xvi) a Toll-like receptor (TLR) transmembrane helix, (xvii) a TLR C-terminal cytoplasmic signaling domain, (xviii) an oligomerization domain, (xix) a signal domain, (xx) an anchor domain, (xxi) a collagen-like domain, (xxii) a fibrinogen-like domain, (xxiii) an immunoglobulin domain, (xxiv) an immunoglobulin-like domain, and (xxv) a sequence variant having at least 85% sequence identity to any one of (xi)-(xxiv).

In certain aspects of this embodiment, the at least one additional domain is an immunoglobulin domain. For example, the immunoglobulin domain may comprise the amino acid sequence of SEQ ID NO:12 or a sequence variant thereof having at least 85% sequence identity to SEQ ID NO:12.

In certain aspects of this embodiment, the collectin microbe-binding domain comprises the CRD of MBL or a sequence variant thereof having at least 85% sequence identity to the CRD of MBL. For example, the CRD of MBL may comprise the amino acid sequence of any one of SEQ ID NOs:1, 2, 3, 4, and 5 or a sequence variant thereof having at least 85% sequence identity to any one of SEQ ID NOs:1, 2, 3, 4, and 5.

In certain aspects of this embodiment, the MTM comprises the CRD of MBL or a sequence variant thereof having at least 85% sequence identity to the CRD of MBL and an immunoglobulin domain. For example, the CRD of MBL may comprise the amino acid sequence of any one of SEQ ID NOs:1, 2, 3, 4, and 5 or a sequence variant thereof having at least 85% sequence identity to any one of SEQ ID NOs:1, 2, 3, 4, and 5, and an immunoglobulin domain comprising the amino acid sequence of SEQ ID NO12: or a sequence variant thereof having at least 85% sequence identity to SEQ ID NO:12.

In certain aspects of this embodiment, the MTMs are collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9. FcMBL MTMs comprise a mannose-binding ligand (MBL) linked to the Fc domain of human IgG (Fc).

In a second embodiment, the invention is directed to a composition comprising one or more of the MTMs of the invention. In certain aspects of this embodiment, the composition comprises at least one naturally-occurring MTM. In certain other aspects of this embodiment, the composition comprises one collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9. In certain other aspects of this embodiment, the composition comprises at least two collectin-based engineered MTMs, wherein one of the collectin-based engineered MTMs is an FcMBL of SEQ ID NO:6, 7, 8 or 9, or a sequence variant thereof having at least 85% sequence identity to SEQ ID NO:6, 7, 8 or 9. In certain other aspects of this embodiment, the composition comprises one naturally-occurring collectin MTM (e.g. MBL) and one collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9. In certain other aspects of this embodiment, the composition comprises one naturally-occurring collectin MTM (e.g. MBL) and at least two collectin-based engineered MTMs, wherein one of the collectin-based engineered MTMs is an FcMBL of SEQ ID NO:6, 7, 8 or 9, or a sequence variant thereof having at least 85% sequence identity to SEQ ID NO:6, 7, 8 or 9.

In certain aspects of this embodiment, the composition further comprises a carrier or diluent, such as an aqueous solution comprising sodium acetate having a pH of about 3.2

In a third embodiment, the invention is directed to a composition according to any of the preceding aspects or embodiments, further comprising a therapeutic agent, for example, one or more antimicrobial agents.

In a fourth embodiment, the invention is directed to a composition according to any of the preceding aspects or embodiments, wherein at least a portion of the composition is encapsulated in at least one liposome.

In a fifth embodiment, the invention is directed to a composition according to any of the preceding aspects or embodiments, wherein the composition comprises an aerosol, a rinse, a spray, a cream, a powder, or an ointment that can be administered nasally, orally, and/or ocularly.

In a sixth embodiment, the invention is directed polynucleotides comprising nucleotide sequences encoding the MTMs of the invention, as well as complementary strands thereof; cloning and expression vectors comprising the polynucleotides; cells containing the polynucleotides and/or the cloning and expression vectors; and methods of producing the MTMs of the invention using the cells containing the polynucleotides and/or expression vectors.

Non-limiting examples of polynucleotides comprising nucleotide sequences encoding MTMs of the invention include those encoding full-length human MBL (SEQ ID NO:1), mature human MBL without the signal sequence (e.g. SEQ ID NO:2), a truncated human MBL that retains microbe surface-binding (e.g. SEQ ID NO:3), the carbohydrate recognition domain (CRD) of human MBL (e.g. SEQ ID NO:4), the neck and carbohydrate recognition domain of human MBL (e.g. SEQ ID NO:5), and the FcMBLs of SEQ ID NOs: 6, 7, 8 and 9, and sequence variants thereof having at least 85% sequence identity to any of these polynucleotides.

Suitable cells containing the polynucleotides and/or the cloning and expression vectors may be mammalian or non-mammalian cells, including, but not limited to, E. coli and insect cells. The methods of producing the MTMs defined herein comprise culturing the cells under conditions promoting expression of the MTMs encoded by the polynucleotides and expression vectors, and recovering the MTMs from the cells or cell cultures.

### Diagnostic Devices

The MTMs and compositions of the invention may also be used in diagnostic devices (and related methods) to detect and/or identify microbes and microbial components in a sample.

Thus, and in a seventh embodiment, the invention is directed to a diagnostic device comprising one or more MTMs of the invention. The diagnostic device may be used, for example, in the detection of a microbe or a microbial component in a sample. The diagnostic devices of the invention or at least a component thereof will be coated with MTMs or otherwise display MTMs on a surface of the device or component thereof.

The diagnostic devices of the invention include, but are not limited to, the following: dipsticks, test strips, and any other sample collection devices known in the art. At least one surface of the device is coated with MTMs of the invention, or otherwise display MTMs such that the MTMs are exposed to a sample, such as a biological sample, under conditions permitting binding of microbes or microbial components in the sample by the MTMs.

Alternatively, or in addition, the diagnostic devices of the invention comprise at least one component that is coated with MTMs of the invention, or otherwise display MTMs such that the MTMs are exposed to a sample, such as a biological sample, under conditions permitting binding of microbes or microbial components in the sample by the MTMs. Such components include, but are not limited to, supports (e.g. graphene), beads (e.g. gold particles), particles (including nanoparticles, microparticles, polymer microbeads, magnetic microbeads, and the like), filters, fibers, screens, mesh, tubes, hollow fibers, scaffolds, plates, channels, magnetic materials, medical apparatuses (e.g., needles or catheters) or implants, filtration devices or membranes, cartridges (e.g. hollow fiber cartridges), microfluidic devices, mixing elements (e.g., spiral mixers), and other substrates commonly utilized in assay formats, and any combinations thereof.

In some aspects, the support is a magnetic support. In some aspects, the magnetic support is a superparamagnetic support. In some aspects, the magnetic support comprises a magnetic bead, a superparamagnetic bead, or a magnetic microbead. In some aspects, the support is a gold, silver, or graphene, for example, for use in SPR and LSPR.

The diagnostic devices of the invention may be used in a wide variety of diagnostic applications including, but not limited to, methods of detecting the presence of a microbe or microbial component in the bodily fluid of a subject. Such methods include contacting a bodily fluid of the subject with a diagnostic device of the invention under conditions that permit binding of microbes or microbial components by MTMs displayed by the diagnostic device, thus detecting microbes or microbial components in the bodily fluid of the subject. In one aspect, the microbe is a bacteria. In another aspect, the microbe is a virus. In further aspect, the microbe is a fungus. Optionally, such methods can include one or more of the following additional steps: (i) quantifying the amount of microbe or microbial component in the bodily fluid; (ii) identifying the microbe in the bodily fluid. Suitable means for identifying the microbe are discussed below.

The invention is thus directed to diagnostic devices comprising at least one component coated with, or otherwise displaying, one or more microbe-targeting molecules (MTMs). In certain aspects, the device is a dipstick or a test strip. In certain aspects, the component is selected from the group consisting of a supports (e.g. graphene), beads (e.g. gold particles), particles (including nanoparticles, microparticles, polymer microbeads, magnetic microbeads, and the like), filters, fibers, screens, mesh, tubes, hollow fibers, scaffolds, plates, channels, magnetic materials, medical apparatuses (e.g., needles or catheters) or implants, filtration devices or membranes, cartridges (e.g. hollow fiber cartridges), microfluidic devices, mixing elements (e.g., spiral mixers), and any combinations thereof. In certain aspects, the support is graphene. In certain aspects, the particle is one or more of nanoparticles, microparticles, polymer microbeads, and magnetic microbeads. In certain aspects, the support is a filter. In certain aspects, the diagnostic device comprises a first MTM and a second MTM, wherein the first and second MTMs have different binding specificities, and wherein the first MTM is affixed to the component in a first predetermined pattern and the second MTM is affixed to the component in a second predetermined pattern.

The invention is also directed to a method of detecting a microbe in a sample, comprising contacting a sample suspected of containing a microbe with a diagnostic device of the invention under conditions permitting binding of a microbe or a component of a microbe by MTMs displayed by the at least one component of the diagnostic device, thereby detecting a microbe in a sample.

The invention is also directed to a method of detecting a microbial infection in a subject, comprising contacting a biological sample of a subject suspected of having a microbial infection with a diagnostic device of the invention under conditions permitting binding of microbes or microbial components by MTMs displayed by the at least one component of the diagnostic device, thereby detecting a microbial infection in the subject.

The invention is also directed to a method of diagnosing a microbial infection in a subject, comprising contacting a biological sample of a subject suspected of having a microbial infection with a diagnostic device of the invention under conditions permitting binding of microbes or microbial components by MTMs displayed by the at least one component of the diagnostic device, thereby diagnosing a microbial infection in the subject.

In each of these methods, the methods may further comprise identifying the microbe bound by the MTMs.

In each of these methods, the sample may be a biological sample. For example, the biological sample may be blood.

In each of these methods, the diagnostic device may comprise two or more MTMs having different binding specificities.

In specific, non-limiting examples of a diagnostic device of the invention, the one or more MTMs may be a collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9.

In selected aspects of these examples, the device further comprises at least one naturally-occurring MTM.

### Therapeutic Devices

The MTMs and compositions of the invention may further be used in therapeutic devices (and related methods) to treat microbial infections and diseases and related conditions in a subject.

Thus, and in an eighth embodiment, the invention is directed to a therapeutic device comprising one or more MTMs of the invention. The therapeutic device may be used, for example, in the treatment of a microbial infection, disease or related condition in a subject. The therapeutic devices of the invention or at least a component thereof will be coated with MTMs or otherwise display MTMs on a surface of the device or component thereof.

The therapeutic devices of the invention include, but are not limited to, the following: oxygenation devices, extracorporeal devices (e.g. ECMO devices), blood pump devices, heart-lung devices, dialysis devices, drainage devices, blood transfusion devices, infusion devices, temperature management devices, pressure management devices, plasma separators, hemoperfusion cartridges, adsorbent devices, monitoring devices, cytokine reduction systems, pathogen reduction systems, PAMP reduction systems, respiration devices, ventilation devices, and catheters or tubes used in a medical procedure.

At least one surface of the therapeutic device is coated with MTMs of the invention, or otherwise displays MTMs such that the MTMs are exposed to a sample, such as a biological sample, under conditions permitting binding of microbes or microbial components in the sample by the MTMs.

Alternatively, or in addition, the therapeutic devices of the invention comprise at least one component that is coated with MTMs of the invention, or otherwise display MTMs such that the MTMs are exposed to a sample, such as a biological sample, under conditions permitting binding of microbes or microbial components in the sample by the MTMs. Such components include, but are not limited to, supports (e.g. graphene), beads (e.g. gold particles), particles (including nanoparticles, microparticles, polymer microbeads, magnetic microbeads, and the like), filters, fibers, screens, mesh, tubes, hollow fibers, scaffolds, plates, channels, magnetic materials, medical apparatuses (e.g., needles or catheters) or implants, dipsticks or test strips, filtration devices or membranes, cartridges (e.g. hollow fiber cartridges), microfluidic devices, mixing elements (e.g., spiral mixers), extracorporeal devices, and other substrates commonly utilized in therapeutic applications, and any combinations thereof. In some aspects, the therapeutic device or component thereof is a solid substrate, such as a filter or cartridge.

The therapeutic devices of the invention may be used in a wide variety of therapeutic applications including, but not limited to, methods of treating microbial infections in a subject. Such methods include contacting a bodily fluid of the subject with a therapeutic device of the invention under conditions that permit binding of microbes by MTMs displayed by the therapeutic device, thus reducing the amount of microbes in the bodily fluid of the subject. In one aspect, the microbial infection is a bacterial infection. In another aspect, the microbial infection is a viral infection. In further aspect, the microbial infection is a fungal infection. Such methods can be used to treat infectious diseases.

The therapeutic devices of the invention may be used in therapeutic applications that remove microbial components from a subject. Such subjects do not have an active microbial infection, but may be suffering from the effects of the continued presence of microbial components. For example, clearing residual PAMPs (e.g. DAMPs) from the blood could decrease the amount of organ damage caused by microbial components through inflammation. Such methods include contacting a bodily fluid of the subject with a therapeutic device of the invention under conditions that permit binding of microbial components by MTMs displayed by the therapeutic device, thus reducing the amount of microbial components in the bodily fluid of the subject. Such methods can also be used to “scrub” non-self substances from the blood, with the “clean” blood being returned to the subject or donating for use in a different subject or assayed. In such applications, the therapeutic devices could be the filter of a hemodialysis device and/or even simply the tubing or flow path that conveys the blood through the device.

It should be understood that the therapeutic devices of the invention may also be used in therapeutic applications that are not directed to the capture of microbes or microbial components. For example, sterile inflammation is a type of pathogen-free inflammation caused by mechanical trauma, ischemia, stress or environmental conditions such as ultra-violet radiation. These damaging factors induce the secretion of molecular agents collectively termed danger-associated molecular patterns (DAMPs). DAMPs are recognized by immune receptors, such as toll-like receptors (TLRs) and NOD-like receptor family, pyrin domain containing 3 (NLRP3), expressed by sentinel cells of the immune system. The therapeutic devices of the invention can be used to reduce and/or remove DAMPs from the blood of a subject. Such devices include a filter having at least one surface coated with or otherwise displaying MTMs of the invention. Methods using these devices include contacting a bodily fluid of the subject with a therapeutic device of the invention under conditions that permit binding of DAMPs by MTMs displayed by the therapeutic device, thus reducing the amount of DAMPs in the bodily fluid of the subject.

The invention is thus directed to therapeutic devices comprising at least one component coated with, or otherwise displaying, one or more MTMs. In certain aspects, the device is a filtration device, an oxygenation device, an extracorporeal device, a dialysis device, an infusion device, a drainage device, or a catheter or tube used in a surgical procedure. In certain aspects, the component is selected from the group consisting of a supports (e.g. graphene), beads (e.g. gold particles), particles (including nanoparticles, microparticles, polymer microbeads, magnetic microbeads, and the like), filters, fibers, screens, mesh, tubes, hollow fibers, scaffolds, plates, channels, magnetic materials, medical apparatuses (e.g., needles or catheters) or implants, filtration devices or membranes, cartridges (e.g. hollow fiber cartridges), microfluidic devices, mixing elements (e.g., spiral mixers), and any combinations thereof. In certain aspects, the component is a filter.

The invention is also directed to a method of treating a microbial infection in a subject, comprising contacting a bodily fluid of a subject having a microbial infection with a therapeutic device of the invention under conditions permitting binding of microbes by MTMs displayed by the at least one component of the therapeutic device, thereby treating a microbial infection in the subject.

The invention is also directed to a method of reducing microbes or microbial components in a bodily fluid of a subject, comprising contacting a bodily fluid of a subject with a therapeutic device of the invention under conditions permitting binding of microbes or microbial components by MTMs displayed by the at least one component of the therapeutic device, thereby reducing microbes or microbial components in a bodily fluid of the subject.

In each of these methods, the bodily fluid may be blood.

In each of these methods, the therapeutic device may comprise two or more MTMs having different binding specificities.

In specific, non-limiting, examples of a therapeutic device of the invention, the one or more MTMs may be a collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9.

In selected aspects of any of these examples, the device further comprises at least one naturally-occurring MTM.

### Filtration Devices

The MTMs and compositions of the invention may further be used in filtration devices (and related methods).

Thus, and in a ninth embodiment, the invention is directed to a filtration device comprising one or more MTMs of the invention. The filtration devices of the invention can be used to remove microbes and microbial components from a fluid.

The filtration devices of the invention will be coated with MTMs or otherwise display MTMs on a surface of the device. Thus, at least one surface of the device, such as a filter, is coated with MTMs of the invention, or otherwise displays MTMs such that the MTMs are exposed to a fluid under conditions permitting binding of microbes or microbial components in the fluid by the MTMs.

The filtration devices of the invention include those comprising a paper filter, e.g., cellulose, or a membrane filter, such as regenerated cellulose, cellulose acetate, nylon, PTFE, polypropylene, polyester, polyethersulfone, polyarylethersulfone, polycarbonate, and polyvinylpyrolidone. The filter may be coated with MTMs.

Alternatively, or in addition, the filtration devices of the invention comprise at least one component that is coated with MTMs of the invention, or otherwise display MTMs such that the MTMs are exposed to a sample, such as a biological sample, under conditions permitting binding of microbes or microbial components in the sample by the MTMs. Such components include, but are not limited to, supports (e.g. graphene), beads (e.g. gold particles), particles (including nanoparticles, microparticles, polymer microbeads, magnetic microbeads, and the like), fibers, screens, mesh, tubes, hollow fibers, scaffolds, plates, channels, magnetic materials, medical apparatuses (e.g., needles or catheters) or implants, dipsticks or test strips, filtration devices or membranes, cartridges (e.g. hollow fiber cartridges), microfluidic devices, mixing elements (e.g., spiral mixers), extracorporeal devices, and other substrates commonly utilized in therapeutic applications, and any combinations thereof. In some aspects, the therapeutic device or component thereof is a solid substrate, such as a filter or cartridge.

The filtration devices of the invention may be used in a wide variety of applications including, but not limited to, methods of removing microbes or microbial components from a fluid. Such methods include contacting a bodily fluid of the subject with a filtration device of the invention under conditions that permit binding of microbes by MTMs displayed by the filtration device, thus reducing the amount of microbes in the bodily fluid of the subject. In one aspect, the microbial infection is a bacterial infection. In another aspect, the microbial infection is a viral infection. In further aspect, the microbial infection is a fungal infection. Such methods can be used to treat infectious diseases.

The filtration devices of the invention may also be used in non-biological applications. For example, the filtration devices of the invention may be used in a method that removes microbes or microbial components from an agricultural product, a food or beverage, an environmental sample, a pharmaceutical sample, etc.

The invention is thus directed to filtration devices comprising at least one component coated with, or otherwise displaying, one or more MTMs. In certain aspects, the device comprises a paper filter, e.g., cellulose, or a membrane filter, such as regenerated cellulose, cellulose acetate, nylon, PTFE, polypropylene, polyester, polyethersulfone, polyarylethersulfone, polycarbonate, and polyvinylpyrolidone. The filter may be coated with MTMs.

The invention is also directed to a method of filtering a fluid, comprising contacting a fluid with a filtration device of the invention under conditions permitting binding of microbes in the fluid by MTMs displayed by the at least one component of the filtration device, thereby filtering a fluid.

The invention is also directed to a method of reducing microbes or microbial components in a fluid, comprising contacting a fluid with a filtration device of the invention under conditions permitting binding of microbes or microbial components by MTMs displayed by the at least one component of the filtration device, thereby reducing microbes or microbial components in a fluid.

In each of these methods, the fluid may be blood.

In each of these methods, the filtration device may comprise two or more MTMs having different binding specificities.

In specific, non-limiting, examples of a filtration device of the invention, the one or more MTMs may be a collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9.

In selected aspects of any of these examples, the device further comprises at least one naturally-occurring MTM.

### Systems and Methods for Treating Fluids

The therapeutic and filtration devices of the invention may be used in practical applications, such as systems and methods for treating fluids, including pathogen filtration from fluids.

Thus, and in a tenth embodiment, the invention is directed to a system comprising at least one filtration device, as defined herein, and optionally at least one therapeutic device, as defined herein, wherein the system is configured to receive a fluid from a fluid source. In the avoidance of doubt, the systems of the invention include (i) a system comprising at least one filtration device, and (ii) a system comprising at least one filtration device and at least one therapeutic device.

In certain aspects of this embodiment, the at least one therapeutic device is an oxygenation device, an ECMO device, a blood pump device, a heart-lung device, a dialysis device, a drainage device, a blood transfusion device, an infusion device, a temperature management device, a pressure management device, a filtration device, a plasma separator, a hemoperfusion cartridge, an adsorbent device, a monitoring device, a cytokine reduction system, a pathogen reduction system, a PAMP reduction system, a respiration device, a ventilation device, or catheters or tubes used in a medical procedure.

In certain aspects of this embodiment, the therapeutic device and the filtration device form a flow pathway, where the filtration device is in series with the therapeutic device.

In certain aspects of this embodiment, the therapeutic device and the filtration device form a flow pathway, where the filtration device is in parallel with the therapeutic devices.

In certain aspects of this embodiment, the therapeutic devices comprises the filtration device.

In certain aspects of this embodiment, the therapeutic device comprises the filtration device, where the filtration device comprises a coating on a component of the therapeutic device.

In certain aspects of this embodiment, the therapeutic device comprises the filtration device, where the filtration device comprises one or more substrates.

In certain aspects of this embodiment, the fluid source is at a higher pressure than a fluid deposit.

In certain aspects of this embodiment, the fluid source is a patient or a fluid storage system.

In certain aspects of this embodiment, the fluid source is an artery or a vein of the patient.

In certain aspects of this embodiment, the fluid is blood and the fluid source is the artery of the patient and the fluid deposit is a vein of the patient.

In certain aspects of this embodiment, the fluid is blood and the fluid source is the vein of the patient and the fluid deposit is the vein of the patient.

In certain aspects of this embodiment, the fluid deposit is a patient, a different patient, or a filtered fluid storage system.

In certain aspects of this embodiment, the system further comprises a pump configured to move the fluid from the fluid source to the system and to move the fluid from the system to a fluid deposit.

In certain aspects of this embodiment, the system further comprises an additive configured to attach the MTM to a pathogen or fragment thereof.

In certain aspects of this embodiment, the filtration device comprises one or more substrates. The MTM may attach to the substrates though a covalent linking process. As non-limiting examples, the substrates comprise one or more of a bead, a plate, a fiber, a hollow fiber, a filter, a tube, and a membrane. The substrates may comprise a coating configured to reduce thrombosis.

In certain aspects of this embodiment, the system further comprises a temperature control system configured to control the temperature of the fluid.

In certain aspects of this embodiment, the system further comprises a flow-control system configured to control a flow rate of the fluid within the system.

In certain aspects of this embodiment, the fluid is one or more of mucous, phlegm, saliva, sputum, blood, plasma, serum, serum derivatives, bile, sweat, amniotic fluid, menstrual fluid, mammary fluid, peritoneal fluid, interstitial fluid, urine, semen, synovial fluid, interocular fluid, a joint fluid, an articular fluid, or cerebrospinal fluid.

In a ninth embodiment, the invention is directed to methods for using the systems of the invention, as defined herein, in the removal of microbes and microbial components from a fluid.

For example, the invention includes a method for filtering and/or treating a fluid comprising: providing a fluid from a fluid source to a system of the invention, filtering and/or treating the fluid in the system, and providing the filtered and/or treated fluid to a fluid deposit.

In certain aspects of this example, treating the fluid in the system comprises filtering the fluid of one or more microbes or microbial components using a filtration device.

In certain aspects of this example, treating the fluid in the system further comprises providing a therapy to the fluid using at least one therapeutic device.

In certain aspects of this example, providing a therapy to a fluid comprises one or more of oxygenating the fluid, removing carbon dioxide from the fluid, adding an agent such as a drug to the fluid and infusing the fluid to a subject, or removing the fluid from a subject.

In certain aspects of this example, the method further comprises analyzing the fluid. For example, the analyzing of the fluid may comprise detecting and/or identifying one or more microbes or microbial components present in the fluid.

In certain aspects of this example, the method further comprises regulating a temperature of the fluid.

In certain aspects of this example, the method further comprises regulating a flow rate of the fluid through the system.

As another example, the invention includes a method for filtering and/or treating a fluid comprising: removing a fluid from a fluid source, providing the fluid to a system, filtering and/or treating the fluid in the system, and providing the filtered and/or treated fluid to a fluid deposit.

In certain aspects of this example, treating the fluid in the system comprises filtering the fluid of one or more microbes or microbial components using a filtration device.

In certain aspects of this example, treating the fluid in the system further comprises providing a therapy to the fluid using at least one therapeutic device.

In certain aspects of this example, providing a therapy to a fluid comprises one or more of oxygenating the fluid, removing carbon dioxide from the fluid, adding an agent such as a drug to the fluid and infusing the fluid to a subject, or removing the fluid from a subject.

In certain aspects of this example, the method further comprises analyzing the fluid. For example, the analyzing of the fluid comprises detecting and/or identifying one or more microbes or microbial components present in the fluid.

In certain aspects of this example, the method further comprises regulating a temperature of the fluid.

In certain aspects of this example, the method further comprises regulating a flow rate of the fluid through the system.

### BRIEF DESCRIPTION OF THE DRAWINGS

FIG. 1. Diagram showing the structure of an exemplary collectin (MBL).

FIG. 2. An exemplary filtration system 101 of the invention is shown. The system includes one or more cartridges 103 comprising one or more MTMs of the invention.

FIG. 3. An exemplary system 201 of the invention is shown. The system includes a filtration device 203 comprising one or more substrate-bound MTMs with target-binding domains and a one or more therapeutic devices 231. The system is configured in a flow pathway, shown as a circuit, where the filtration device 203 and therapeutic device 231 are in series. The system can be configured such that the fluid can flow through the filtration device 203 first or the therapeutic device 231 first.

FIG. 4. An exemplary system 301 of the invention is shown. The system includes a filtration device 303 comprising one or more substrate-bound MTMs with target-binding domains and one or more therapeutic devices 331. The system is configured as a flow pathway, shown as a circuit, similar to system 201 of FIG. 3, however the filtration device 303 and therapeutic device 331 are in parallel.

FIG. 5 illustrates a first view of an exemplary cartridge filtration device of the invention.

FIG. 6 illustrates a second view of an exemplary cartridge filtration device of the invention.

### DETAILED DESCRIPTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

As summarized above, the present invention is generally directed to (i) microbe-targeting molecules (MTMs) and engineered MTMs that have the shared characteristic of binding to one or more microbe-associated molecular patterns (MAMPs), (ii) polynucleotides, cloning and expression vectors, and cells comprising the same, (iii) diagnostic, therapeutic and filtration devices comprising the MTMs, and to methods of using such devices in diagnosis, treatment and filtration, and (iv) systems and methods for treating fluids, including pathogen filtration from fluids, using the MTMs and devices of the invention.

The diagnostic devices of the invention can be used, for example, in the detection and/or identification of microbes in a biological sample. The therapeutic devices of the invention can be used, for example, in the treatment of microbial infections and diseases and related conditions in a subject. The filtration devices of the invention can be used, for example, to isolate microbes and microbial components from a sample. The systems and methods for treating fluids can be used, for example, in biological and non-biological applications.

The important characteristic of the MTMs used in the devices, systems and methods of the invention is that these constructs contact and bind microbes and microbial components in a sample based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

As used herein, “MTM” and “engineered MTM” refers to any of the molecules described herein (or described in patents or patent application incorporated by reference) that can bind to a microbe or microbe component. Unless the context indicates otherwise, the term “MTM” is used to describe all MTMs of the invention, both naturally-occurring and engineered forms of these constructs. The terms “microbe-targeting molecule” and “microbe-binding molecule” are used interchangeably herein.

### MAMPs

Before discussing the MTMs of the invention, it will be helpful to understand the molecules to which the MTMs bind. As indicated above, each of the MTMs of the invention binds to at least one microbe-associated molecular pattern (MAMP). Some MTMs bind at least two, at least three, at least four, at least five, or more than five MAMPs.

As used herein and throughout the specification, the term “microbe-associated molecular patterns” or “MAMPs” refers to molecules, components or motifs associated with or secreted or released by microbes or groups of microbes (whole and/or lysed and/or disrupted) that are generally recognized by corresponding pattern recognition receptors (PRRs) of the MTM microbe-binding domains defined herein. In some aspects, the MAMPs encompass molecules associated with cellular components released during cell damage or lysis. Examples of MAMPs include, but are not limited to, microbial carbohydrates (e.g., lipopolysaccharide or LPS, mannose), endotoxins, microbial nucleic acids (e.g., bacterial, fungal or viral DNA or RNA; e.g., nucleic acids comprising a CpG site), microbial peptides (e.g., flagellin), peptidoglycans, lipoteichoic acids, N-formylmethionine, lipoproteins, lipids, phospholipids or their precursors (e.g., phosphochloline), and fungal glucans.

In some aspects, microbe components comprise cell wall or membrane components known as pathogen-associated molecular patterns (PAMPs) including lipopolysaccharide (LPS) endotoxin, lipoteichoic acid, and attached or released outer membrane vesicles. In some aspects, a microbe comprises a host cell membrane and a pathogen component or a PAMP.

In some aspects, microbe components comprise damage-associated molecular patterns (DAMPs), also known as danger-associated molecular patterns, danger signals, and alarmin. These biomolecules can initiate and sustain a non-infectious inflammatory response in a subject, in contrast to PAMPs which initiate and sustain an infectious pathogen-induced inflammatory response. Upon release from damaged or dying cells, DAMPs activate the innate immune system through binding to pattern recognition receptors (PRRs). DAMPs include portions of nuclear and cytosolic proteins, ECM (extracellular matrix), mitochondria, granules, ER (endoplasmic reticulum), and plasma membrane.

In some aspects, MAMPs include carbohydrate recognition domain (CRD)-binding motifs. As used herein, the term “carbohydrate recognition domain (CRD)-binding motifs” refers to molecules or motifs that are bound by a molecule or composition comprising a CRD (i.e. CRDs recognize and bind to CRD-binding motifs). As used herein, the term “carbohydrate recognition domain” or “CRD” refers to one or more regions, at least a portion of which, can bind to carbohydrates on a surface of microbes or pathogens. In some aspects, the CRD can be derived from a lectin, as described herein. In some aspects, the CRD can be derived from a mannan-binding lectin (MBL). Accordingly, in some aspects, MAMPs are molecules, components or motifs associated with microbes or groups of microbes that are recognized by lectin-based MTMs (collectin-based MTMs) described herein that have a CRD domain. In one embodiment, MAMPs are molecules, components, or motifs associated with microbes or groups of microbes that are recognized by mannan-binding lectin (MBL).

In some aspects, MAMPs are molecules, components or motifs associated with microbes or groups of microbes that are recognized by a C-reactive protein (CRP)-based MTMs (collectin-based MTMs).

For clarity, MAMPs as used herein includes microbe components such as MAMPs, PAMPs and DAMPs as defined above.

When necessary, and unless otherwise detectable without pre-treatment, MAMPs can be exposed, released or generated from microbes in a sample by various sample pretreatment methods. In some aspects, the MAMPs can be exposed, released or generated by lysing or killing at least a portion of the microbes in the sample. Without limitations, any means known or available to the practitioner for lysing or killing microbe cells can be used. Exemplary methods for lysing or killing the cells include, but are not limited to, physical, mechanical, chemical, radiation, biological, and the like. Accordingly, pre-treatment for lysing and/or killing the microbe cells can include application of one or more of ultrasound waves, vortexing, centrifugation, vibration, magnetic field, radiation (e.g., light, UV, Vis, IR, X-ray, and the like), change in temperature, flash-freezing, change in ionic strength, change in pH, incubation with chemicals (e.g. antimicrobial agents), enzymatic degradation, and the like.

### Microbes

As used herein, the term “microbe”, and the plural “microbes”, generally refers to microorganism(s), including bacteria, virus, fungi, parasites, protozoan, archaea, protists, e.g., algae, and a combination thereof. The term “microbe” encompasses both live and dead microbes. The term “microbe” also includes pathogenic microbes or pathogens, e.g., bacteria causing diseases such as sepsis, plague, tuberculosis and anthrax; protozoa causing diseases such as malaria, sleeping sickness and toxoplasmosis; and fungi causing diseases such as ringworm, candidiasis or histoplasmosis.

In some aspects, the microbe is a human pathogen, in other words a microbe that causes at least one disease in a human.

In some aspects, the microbe is a Gram-positive bacterial species, a Gram-negative bacterial species, a Mycobacterium, a fungus, a parasite, protozoa, or a virus. In some aspects, the Gram-positive bacterial species comprises bacteria from the class Bacilli. In some aspects, the Gram-negative bacterial species comprises bacteria from the class Gammaproteobacteria. In some aspects, the Mycobacterium comprises bacteria from the class Actinobacteria. In some aspects, the fungus comprises fungus from the class Saccharomycetes.

In some aspects, the microbe is Staphylococcus aureus, Streptococcus pyogenes, Klebsiella pneumoniae, Pseudomonas aeruginosa, Mycobacterium tuberculosis, Candida albicans, or Escherichia coli. In some aspects, the microbe is S. aureus strain 3518, S. pyogenes strain 011014, K. pneumoniae strain 631, E. coli strain 41949, P. aeruginosa strain 41504, C. albicans strain 1311, or M. tuberculosis strain H37Rv.

In some aspects, the microbe is Bartonella henselae, Borrelia burgdorferi, Campylobacter jejuni, Campylobacter fetus, Chlamydia trachomatis, Chlamydia pneumoniae, Chylamydia psittaci, Simkania negevensis, Escherichia coli (e.g., 0157:H7 and K88), Ehrlichia chafeensis, Clostridium botulinum, Clostridium perfringens, Clostridium tetani, Enterococcus faecalis, Haemophilius influenzae, Haemophilius ducreyi, Coccidioides immitis, Bordetella pertussis, Coxiella burnetii, Ureaplasma urealyticum, Mycoplasma genitalium, Trichomatis vaginalis, Helicobacter pylori, Helicobacter hepaticus, Legionella pneumophila, Mycobacterium tuberculosis, Mycobacterium bovis, Mycobacterium africanum, Mycobacterium leprae, Mycobacterium asiaticum, Mycobacterium avium, Mycobacterium celatum, Mycobacterium celonae, Mycobacterium fortuitum, Mycobacterium genavense, Mycobacterium haemophilum, Mycobacterium intracellulare, Mycobacterium kansasii, Mycobacterium malmoense, Mycobacterium marinum, Mycobacterium scrofulaceum, Mycobacterium simiae, Mycobacterium szulgai, Mycobacterium ulcerans, Mycobacterium xenopi, Corynebacterium diptheriae, Rhodococcus equi, Rickettsia aeschlimannii, Rickettsia africae, Rickettsia conorii, Arcanobacterium haemolyticum, Bacillus anthracia, Bacillus cereus, Lysteria monocytogenes, Yersinia pestis, Yersinia enterocolitica, Shigella dysenteriae, Neisseria meningitides, Neisseria gonorrhoeae, Streptococcus bovis, Streptococcus hemolyticus, Streptococcus mutans, Streptococcus pyogenes, Streptococcus pneumoniae, Staphylococcus aureus, Staphylococcus epidermidis, Staphylococcus pneumoniae, Staphylococcus saprophyticus, Vibrio cholerae, Vibrio parahaemolyticus, Salmonella typhi, Salmonella paratyphi, Salmonella enteritidis, Treponema pallidum, Human rhinovirus, Human coronavirus such as SARS-CoV-2, Dengue virus, Filoviruses (e.g., Marburg and Ebola viruses), Hantavirus, Rift Valley virus, Hepatitis B, C, and E, Human Immunodeficiency Virus (e.g., HIV-1, HIV-2), HHV-8, Human papillomavirus, Herpes virus (e.g., HV-I and HV-II), Human T-cell lymphotrophic viruses (e.g., HTLV-I and HTLV-II), Bovine leukemia virus, Influenza virus, Guanarito virus, Lassa virus, Measles virus, Rubella virus, Mumps virus, Chickenpox (Varicella virus), Monkey pox, Epstein Bahr virus, Norwalk (and Norwalk-like) viruses, Rotavirus, Parvovirus B19, Hantaan virus, Sin Nombre virus, Venezuelan equine encephalitis, Sabia virus, West Nile virus, Yellow Fever virus, causative agents of transmissible spongiform encephalopathies, Creutzfeldt-Jakob disease agent, variant Creutzfeldt-Jakob disease agent, Candida, Cryptcooccus, Cryptosporidium, Giardia lamblia, Microsporidia, Plasmodium vivax, Pneumocystis carinii, Toxoplasma gondii, Trichophyton mentagrophytes, Enterocytozoon bieneusi, Cyclospora cayetanensis, Encephalitozoon hellem, or Encephalitozoon cuniculi, among other viruses, bacteria, archaea, protozoa, and fungi. In yet other aspects, the microbe is a bioterror agent (e.g., B. anthracis, and smallpox).

As used herein, “microbe component” and “microbial component” refer to any part of a microbe such as cell wall components, cell membrane components, cell envelope components, cytosolic components, intracellular components, nucleic acid (DNA or RNA), or organelles in the case of eukaryotic microbes. In some aspects, the microbial component comprises a component from a Gram-positive bacterial species, a Gram-negative bacterial species, a Mycobacterium, a fungus, a parasite, a virus, or any microbe described herein or known in the art.

### Sample

The MTMs defined herein can be used to detect the MAMP of a microbe or a microbial component in a sample. A sample can include but is not limited to, a patient sample, an animal or animal model sample, an agricultural sample, a food and beverage sample, an environmental sample, a pharmaceutical sample, a biological sample, and a non-biological sample. A biological sample can include but is not limited to, cells, tissue, peripheral blood, and a bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The biological sample can be collected from any source, including, e.g., human or animal suspected of being infected or contaminated by a microbe(s). Biological fluids can include a bodily fluid and may be collected in any clinically acceptable manner. Biological fluids can include, but are not limited to, mucous, phlegm, saliva, sputum, blood, plasma, serum, serum derivatives, bile, sweat, amniotic fluid, menstrual fluid, mammary fluid, peritoneal fluid, interstitial fluid, urine, semen, synovial fluid, interocular fluid, a joint fluid, an articular fluid, and cerebrospinal fluid (CSF). A fluid may also be a fine needle aspirate or biopsied tissue. Blood fluids can be obtained by standard phlebotomy procedures and may be separated into components such as plasma for analysis. Centrifugation can be used to separate out fluid components to obtain plasma, buffy coat, erythrocytes, cells, pathogens and other components.

In some aspects, the sample, such as a fluid, may be purified before introduction to a device or a system of the invention. For example, filtration or centrifugation to remove particulates and chemical interference may be used. Various filtration media for removal of particles includes filter paper, such as cellulose and membrane filters, such as regenerated cellulose, cellulose acetate, nylon, PTFE, polypropylene, polyester, polyethersulfone, polyarylethersulfone, polycarbonate, and polyvinylpyrolidone.

Environmental samples include, but are not limited to, air samples, liquid and fluid samples, and dry samples. Suitable air samples include, but are not limited to, an aerosol, an atmospheric sample, and a ventilator discharge. Suitable dry samples include, but are not limited to, soil. Environmental fluids include, for example, saturated soil water, groundwater, surface water, unsaturated soil water; and fluids from industrialized processes such as waste water. Agricultural fluids can include, for example, crop fluids, such as grain and forage products, such as soybeans, wheat, and corn.

Pharmaceutical samples include, but are not limited to, drug material samples and therapeutic fluid samples, for example, for quality control or detection of endotoxins. Suitable therapeutic fluids include, but are not limited to, a dialysis fluid.

### Subject

The present invention, including the devices, systems and methods, may be used in conjunction with a subject. For example, the therapeutic devices may be used in the treatment of a subject, and the systems of the invention may be used to filter pathogens from the blood of a subject. As used herein, a “subject” is a human, a non-human primate, bird, horse, cow, goat, sheep, a companion animal, such as a dog, cat or rodent, or other mammal. The subject may be a patient undergoing treatment for a medical condition.

### MTMs

As summarized above, the invention is directed to diagnostic, therapeutic and filtration devices, and methods of using the devices, where the devices comprise MTMs that bind to one or more MAMPs.

The diagnostic devices of the invention can be used, for example, in the detection and/or identification of microbes and microbe components in a sample. The therapeutic devices of the invention can be used, for example, in the treatment of microbial infections and diseases and related conditions in a subject. The filtration devices of the invention can be used, for example, to isolate microbes and microbial components from a sample.

MTMs distinguish and bind microbes and microbial components from a sample based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

As will be apparent, while the “MTMs” of the invention include naturally-occurring molecules and proteins, the “engineered MTMs” of the invention are those have been manipulated in some manner by the hand of man. As used herein and throughout the specification, the term “engineered MTM” includes any non-naturally-occurring MTM. Engineered MTMs of the invention retain the binding specificity to a MAMP of the wild-type (i.e. naturally-occurring) molecule on which the engineered MTM is based.

The MTMs of the invention are defined based on their binding activity, therefore both naturally-occurring and engineered MTMs will comprise at least one microbe-binding domain, i.e. a domain that recognizes and binds to one or more MAMPs (including, at least two, at least three, at least four, at least five, or more) as described herein. A microbe-binding domain can be a naturally-occurring or a synthetic molecule. In some aspects, a microbe-binding domain can be a recombinant molecule.

Acceptable microbe-binding domains for use in the MTMs of the invention are limited only in their ability to recognize and bind at least one MAMP. In some aspects, the microbe-binding domain may comprise some or all of a peptide; polypeptide; protein; peptidomimetic; antibody; antibody fragment; antigen-binding fragment of an antibody; carbohydrate-binding protein; lectin; glycoprotein; glycoprotein-binding molecule; amino acid; carbohydrate (including mono-; di-; tri- and poly-saccharides); lipid; steroid; hormone; lipid-binding molecule; cofactor; nucleoside; nucleotide; nucleic acid; DNA; RNA; analogues and derivatives of nucleic acids; peptidoglycan; lipopolysaccharide; small molecule; endotoxin; bacterial lipopolysaccharide; and any combination thereof.

In particular aspects, the microbe-binding domain can be a microbe-binding domain of a lectin. An exemplary lectin is mannan binding lectin (MBL) or other mannan binding molecules. Non-limiting examples of acceptable microbe-binding domains also include microbe-binding domains from toll-like receptors, nucleotide oligomerization domain-containing (NOD) proteins, complement receptors, collectins, ficolins, pentraxins such as serum amyloid and C-reactive protein, lipid transferases, peptidoglycan recognition proteins (PGRs), and any combinations thereof. In some aspects, microbe-binding domains can be microbe-binding molecules described in the International Patent Application No. WO 2013/012924, the contents of which are incorporated by reference in their entirety.

The MTMs of the invention will typically have one or more domains in addition to a microbe-binding domain. Such domains include, but are not limited to, an oligomerization domain, a signal domain, an anchor domain, a collagen-like domain, a fibrinogen-like domain, an immunoglobulin domain, and an immunoglobulin-like domain.

Engineered MTMs of the invention include, but are not limited to, MTMs identical to a naturally-occurring MTM but having at least one amino acid change in comparison to the wild-type molecule on which they are based. Such “sequence-variant engineered MTMs” have at least 51, 52, 53, 54, 55, 56, 57, 58, 59, 60, 60, 61, 62, 63, 64, 65, 66, 67, 68, 69, 70, 71, 72, 73, 74, 76, 77, 78, 79, 80, 81, 82, 83, 84, 85, 86, 87, 88, 89, 90, 91, 92, 93, 94, 95, 96, 97, 98 or 99 sequence identity, though in all cases less than 100% sequence identity, to the wild-type molecule on which they are based. The changes may be any combination of additions, insertions, deletions and substitutions, where the altered amino acids may be naturally-occurring or non-naturally-occurring amino acids, and conservative or non-conservative changes.

Engineered MTMs of the invention also include, but are not limited to, MTMs that comprise domains from two or more different MTMs, i.e. fusion proteins. Such “domain-variant engineered MTMs” have domains from 2, 3, 4, 5 or more different proteins. For example, MTMs can be a fusion protein comprising a microbe-binding domain and an oligomerization domain, or a fusion protein comprising a microbe-binding domain and a signal domain, or a fusion protein comprising a microbe-binding domain, an oligomerization domain, and signal domain, to name a few examples. In each case, the domains within a domain-variant engineered MTM are from at least two different proteins. Other examples of such MTMs include fusion proteins comprising at least the microbe-binding domain of a lectin and at least a part of a second protein or peptide, e.g., but not limited, to an Fc portion of an immunoglobulin.

Engineered MTMs of the invention further include, but are not limited to, MTMs that comprise domains from two or more different MTMs, wherein at least one of the domains is a sequence variant of the wild-type domain upon which it is based, i.e. having at least one amino acid change in comparison to the wild-type molecule on which it is based. These “sequence- and domain-variant engineered MTMs” have domains from 2, 3, 4, 5 or more different proteins, and at least one of the domains has at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 sequence identity, though in all cases less than 100% sequence identity, to the wild-type domain on which it is based. The changes may be any combination of additions, insertions, deletions and substitutions, where the altered amino acids may be naturally-occurring or non-naturally-occurring amino acids, and conservative or non-conservative changes.

### Collectin-Based MTMs

The MTMs of the invention include collectin-based MTMs. These MTMs are either naturally-occurring collectin proteins or engineered MTM fusion proteins that comprise at least one collectin microbe-binding domain, such as the lectin carbohydrate-recognition domain (CRD), and at least one additional domain

Collectins (collagen-containing C-type lectins) are a family of collagenous calcium-dependent lectins that function in defense, thus playing an important role in the innate immune system. They are soluble molecules comprising pattern recognition receptors (PRRs) within the microbe-binding domain that recognize and bind to particular oligosaccharide structures or lipids displayed on the surface of microbes, i.e. MAMPs of oligosaccharide origin. Upon binding of collectins to a microbe, clearance of the microbe is achieved via aggregation, complement activation, opsonization, and activation of phagocytosis.

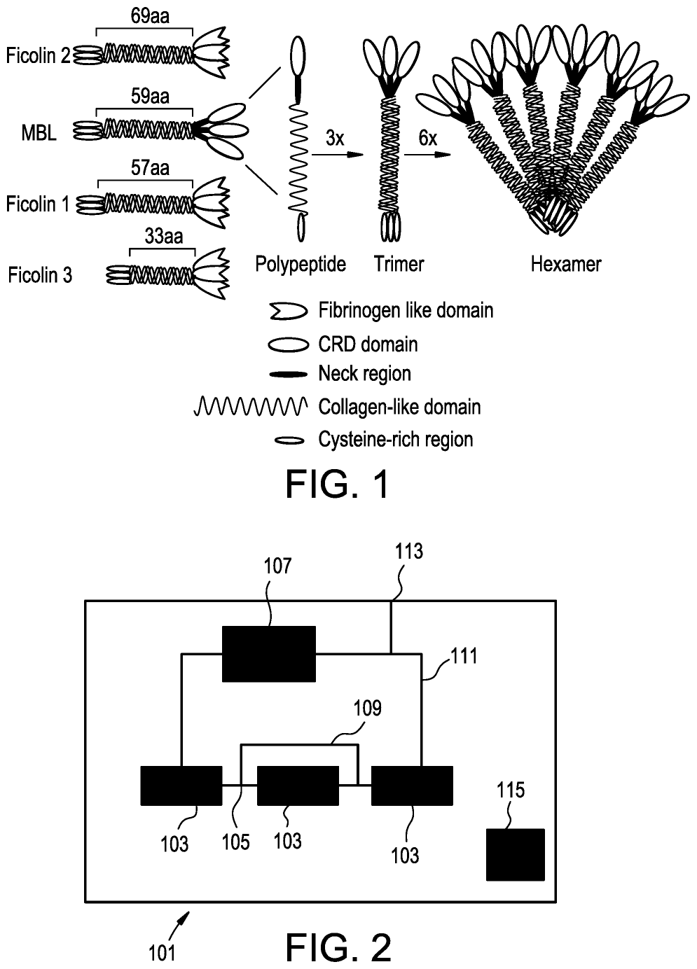

Members of the family have a common structure, characterized by four parts or domains arranged in the following N- to C-terminal arrangement: (i) a cysteine-rich domain, (ii) a collagen-like domain, (iii) a coiled-coil neck domain, and (iv) a microbe-binding domain which includes a C-type lectin domain, also termed the carbohydrate recognition domain (CRD). The functional form of the molecule is a trimer made up of three identical chains. MAMP recognition is mediated by the CRD in presence of calcium. See FIG. 1.

There are currently nine recognized members of the family: (i) mannose-binding lectin (MBL; mannose-binding lectin; e.g. SEQ ID NO:1), (ii) surfactant protein A (SP-A; SEQ ID NO:13), (iii) surfactant protein D (SP-D; SEQ ID NO:14), (iv) collectin liver 1 (CL-L1; SEQ ID NO:15), (v) collectin placenta 1 (CL-P1; SEQ ID NO:16), (vi) conglutinin collectin of 43 kDa (CL-43; SEQ ID NO:17), (vii) collectin of 46 kDa (CL-46; SEQ ID NO:18), (viii) collectin kidney 1 (CL-K1; SEQ ID NO:19), and (ix) conglutinin (SEQ ID NO:20). Each of these proteins is an MTM of the invention.

MBL (Homo sapiens; GenBank: AAH69338.1; SEQ ID NO: 1):
MSLFPSLPLL LLSMVAASYS ETVTCEDAQK TCPAVIACSS PGINGFPGKD
GRDGTKGEKG EPGQGLRGLQ GPPGKLGPPG NPGPSGSPGP KGQKGDPGKS
PDGDSSLAAS ERKALQTEMA RIKKWLTFSL GKQVGNKFFL INGEIMTFEK
VKALCVKFQA SVATPRNAAE NGAIQNLIKE EAFLGITDEK TEGQFVDLTG
NRLTYTNWNE GEPNNAGSDE DCVLLLKNGQ WNDVPCSTSH LAVCEFPI
Surfactant protein A
(SP-A; Homo sapiens; GenBank: AAA36632.1; SEQ ID NO: 13):
MTFKNNTTLS LFRLENSFPL HKGYITTREL HQLALANLIF MENCADLHFP
PQMRKCFPIP GTSCLFCPKT KTLFPVLHLA NQYLCFLCQH KHCFPQTRFP
DSQPSQATSR CVLSIKFTSS IYINSHNYTL ILTCIIVRVL CPPVGCKSYK
DRGHIHLAHP SVPSTCHGSY QLTSTQYIVW ISKYDSNFQH PKLVYSPTEP
AQVEHMQCFL CMCLQREERE ALLLLPRVTI LTRLSAESTD ERDGDSEPVN
AVCRTALAFV PHESNVMLGI HNLLIWLL
Surfactant protein D
(SP-D; Homo sapiens; GenBank: AAB59450.1; SEQ ID NO: 14):
MLLFLLSALV LLTQPLGYLE AEMKTYSHRT MPSACTLVMC SSVESGLPGR
DGRDGREGPR GEKGDPGLPG AAGQAGMPGQ AGPVGPKGDN GSVGEPGPKG
DTGPSGPPGP PGVPGPAGRE GALGKQGNIG PQGKPGPKGE AGPKGEVGAP
GMQGSAGARG LAGPKGERGV PGERGVPGNT GAAGSAGAMG PQGSPGARGP
PGLKGDKGIP GDKGAKGESG LPDVASLRQQ VEALQGQVQH LQAAFSQYKK
VELFPNGQSV GEKIFKTAGF VKPFTEAQLL CTQAGGQLAS PRSAAENAAL
QQLVVAKNEA AFLSMTDSKT EGKFTYPTGE SLVYSNWAPG EPNDDGGSED
CVEIFTNGKW NDRACGEKRL VVCEF
Collectin liver 1 (CL-L1; also known as collectin-10; Homo sapiens;
NCBI Reference Sequence: NP_006429.2; SEQ ID NO: 15):
MNGFASLLRR NQFILLVLFL LQIQSLGLDI DSRPTAEVCA THTISPGPKG
DDGEKGDPGE EGKHGKVGRM GPKGIKGELG DMGDQGNIGK TGPIGKKGDK
GEKGLLGIPG EKGKAGTVCD CGRYRKFVGQ LDISIARLKT SMKFVKNVIA
GIRETEEKFY YIVQEEKNYR ESLTHCRIRG GMLAMPKDEA ANTLIADYVA
KSGFFRVFIG VNDLEREGQY MFTDNTPLQN YSNWNEGEPS DPYGHEDCVE
MLSSGRWNDT ECHLTMYFVC EFIKKKK
Collectin placenta 1
(CL-P1; Homo sapiens; GenBank: BAB72147.1; SEQ ID NO: 16):
MKDDFAEEEE VQSFGYKRFG IQEGTQCTKC KNNWALKFSI ILLYILCALL
TITVAILGYK VVEKMDNVTG GMETSRQTYD DKLTAVESDL KKLGDQTGKK
AISTNSELST FRSDILDLRQ QLREITEKTS KNKDTLEKLQ ASGDALVDRQ
SQLKETLENN SFLITTVNKT LQAYNGYVIN LQQDTSVLQG NLQNQMYSHN
VVIMNLNNLN LTQVQQRNLI TNLQRSVDDT SQAIQRIKND FQNLQQVFLQ
AKKDTDWLKE KVQSLQTLAA NNSALAKANN DTLEDMNSQL NSFTGQMENI
TTISQANEQN LKDLQDLHKD AENRTAIKEN QLEERFQLFE TDIVNIISNI
SYTAHHLRTL TSNLNEVRTT CTDTLTKHTD DLTSLNNTLA NIRLDSVSLR
MQQDLMRSRL DTEVANLSVI MEEMKLVDSK HGQLIKNFTI LQGPPGPRGP
RGDRGSQGPP GPTGNKGQKG EKGEPGPPGP AGERGPIGPA GPPGERGGKG
SKGSQGPKGS RGSPGKPGPQ GPSGDPGPPG PPGKEGLPGP QGPPGFQGLQ
GTVGEPGVPG PRGLPGLPGV PGMPGPKGPP GPPGPSGAVV PLALQNEPTP
APEDNGCPPH WKNFTDKCYY FSVEKEIFED AKLFCEDKSS HLVFINTREE
QQWIKKQMVG RESHWIGLTD SERENEWKWL DGTSPDYKNW KAGQPDNWGH
GHGPGEDCAG LIYAGQWNDF QCEDVNNFIC EKDRETVLSS AL
Conglutinin collectin
(CL-43; Bos taurus; GenBank: AAI16148; SEQ ID NO: 17):
MLPLPLSILL LLTQSQSFLG EEMDVYSEKT LTDPCTVVVC APPADSLRGH
DGRDGKEGPQ GEKGDPGPPG MPGPAGREGP SGRQGSMGPP GTPGPKGEPG
PEGGVGAPGM PGSPGPTGLK GERGTPGPGG AIGPQGPSGA MGPPGLKGDR
GDPGEKGAKG ETSVLEVDTL RQRMRNLEGE VQRLQNIVTQ YRKAVLEPDG
QAVGEKIFKT AGAVKSYSDA EQLCREAKGQ LASPRSSAEN EAVTQLVRAK
NKHAYLSMND ISKEGKFTYP TGGSLDYSNW APGEPNNRAK DEGPENCLEI
YSDGNWNDIE CREERLVICE F
Collectin of 46 kDa
(CL-46; Bos taurus; GenBank: AAM34742; SEQ ID NO: 18):
MLLLPLSVLL LLTQPWRSLG AEMKIYSQKT LANGCTLVVC RPPEGGLPGR
DGQDGREGPQ GEKGDPGSPG PAGRAGRPGP AGPIGPKGDN GSAGEPGPKG
DTGPPGPPGM PGPAGREGPS GKQGSMGPPG TPGPKGDTGP KGGMGAPGMQ
GSPGPAGLKG ERGAPGELGA PGSAGVAGPA GAIGPQGPSG ARGPPGLKGD
RGDPGERGAK GESGLADVNA LKQRVTILEG QLQRLQNAFS RYKKAVLEPD
GQAVGKKIFK TAGAVKSYSD AQQLCREAKG QLASPRSAAE NEAVAQLVRA
KNNDAFLSMN DISTEGKFTY PTGESLVYSN WASGEPNNNN AGQPENCVQI
YREGKWNDVP CSEPLLVICE F
Collectin kidney 1 (CL-K1; also known as collectin-11;
Homo sapiens; GenBank: BAF43301.1; SEQ ID NO: 19):
MRGNLALVGV LISLAFLSLL PSGHPQPAGD DACSVQILVP GLKGDAGEKG
DKGAPGRPGR VGPTGEKGDM GDKGQKGSVG RHGKIGPIGS KGEKGDSGDI
GPPGPNGEPG LPCECSQLRK AIGEMDNQVS QLTSELKFIK NAVAGVRETE
SKIYLLVKEE KRYADAQLSC QGRGGTLSMP KDEAANGLMA AYLAQAGLAR
VFIGINDLEK EGAFVYSDHS PMRTENKWRS GEPNNAYDEE DCVEMVASGG
WNDVACHTTM YFMCEFDKEN M
Conglutinin (Bos taurus; GenBank: BAA04983; SEQ ID NO: 20):
MLLLPLSVLL LLTQPWRSLG AEMTTESQKI LANACTLVMC SPLESGLPGH
DGQDGRECPH GEKGDPGSPG PAGRAGRPGW VGPIGPKGDN GFVGEPGPKG
DTGPRGPPGM PGPAGREGPS GKQGSMGPPG TPGPKGETGP KGGVGAPGIQ
GFPGPSGLKG EKGAPGETGA PGRAGVTGPS GAIGPQGPSG ARGPPGLKGD
RGDPGETGAK GESGLAEVNA LKQRVTILDG HLRRFQNAFS QYKKAVLEPD
GQAVGEKIFK TAGAVKSYSD AEQLCREAKG QLASPRSSAE NEAVTQMVRA
QEKNAYLSMN DISTEGRFTY PTGEILVYSN WADGEPNNSD EGQPENCVEI
FPDGKWNDVP CSKQLLVICE F

The MTMs of the invention also include other collectin-based molecules that bind to one or more MAMPs, e.g. those MTMs comprising at least a portion (e.g. domain) of a lectin-based molecule in the case of an engineered MTM. As used herein, the term “collectin-based molecule” refers to a molecule comprising a microbe-binding domain derived from a collectin, such as a lectin. The term “lectin” as used herein refers to any molecule including proteins, natural or genetically modified (e.g., recombinant), that interacts specifically with saccharides (e.g., carbohydrates). The term “lectin” as used herein can also refer to lectins derived from any species, including, but not limited to, plants, animals (e.g. mammals, such as human), insects and microorganisms, having a desired carbohydrate binding specificity. Examples of plant lectins include, but are not limited to, the Leguminosae lectin family, such as ConA, soybean agglutinin, peanut lectin, lentil lectin, and Galanthus nivalis agglutinin (GNA) from the Galanthus (snowdrop) plant. Other examples of plant lectins are the Gramineae and Solanaceae families of lectins. Examples of animal lectins include, but are not limited to, any known lectin of the major groups S-type lectins, C-type lectins, P-type lectins, and I-type lectins, and galectins. In some aspects, the carbohydrate recognition domain can be derived from a C-type lectin, or a fragment thereof. C-type lectin can include any carbohydrate-binding protein that requires calcium for binding (e.g., MBL). In some aspects, the C-type lectin can include, but are not limited to, collectin, DC-SIGN, and fragments thereof. Without wishing to be bound by theory, DC-SIGN can generally bind various microbes by recognizing high-mannose-containing glycoproteins on their envelopes and/or function as a receptor for several viruses such as HIV and Hepatitis C.

Collectin-based engineered MTMs of the invention are MTMs that comprise at least a microbe-binding domain of a collectin. These MTMs may also include one or more of the other domains of a collectin, e.g. a cysteine-rich domain, a collagen-like domain, and/or a coiled-coil neck domain, as well as one or more domains not typically found in a collectin, such as an oligomerization domain, a signal domain, an anchor domain, a collagen-like domain, a fibrinogen-like domain, an immunoglobulin domain, and/or an immunoglobulin-like domain. When a collectin-based engineered MTM has each of the domains of a wild-type collectin, the MTM will be a sequence-variant engineered MTM as defined above. When a collectin-based engineered MTM has fewer that all of the domains of a wild-type collectin, the MTM will be a domain-variant engineered MTM or a sequence- and domain-variant engineered MTM as defined above.

Collectin-based engineered MTMs comprise a microbe-binding domain derived from at least one carbohydrate-binding protein selected from the group consisting of: MBL; SP-A; SP-D; CL-L1, CL-P1; CL-34; CL-46; CL-K1, conglutinin; maltose-binding protein; arabinose-binding protein; glucose-binding protein; Galanthus nivalis agglutinin; peanut lectin; lentil lectin; DC-SIGN; and C-reactive protein; and any combinations thereof.

### MBL

In particular aspects and embodiments of the invention, the MTM may be MBL, whether full-length human MBL (SEQ ID NO:1), mature human MBL without the signal sequence (e.g. SEQ ID NO:2), a truncated human MBL that retains microbe surface-binding (e.g. SEQ ID NO:3), the carbohydrate recognition domain (CRD) of human MBL (e.g. SEQ ID NO:4), or the neck and carbohydrate recognition domain of human MBL (e.g. SEQ ID NO:5), whether used alone or in combination with a second protein in the form of a fusion protein, such as a FcMBL protein as defined herein.

The amino acid sequence of full-length human MBL (SEQ ID NO:1; GenBank: AAH69338.1) is:

MSLFPSLPLL LLSMVAASYS ETVTCEDAQK TCPAVIACSS
PGINGFPGKD GRDGTKGEKG EPGQGLRGLQ GPPGKLGPPG
NPGPSGSPGP KGQKGDPGKS PDGDSSLAAS ERKALQTEMA
RIKKWLTFSL GKQVGNKFFL INGEIMTFEK VKALCVKFQA
SVATPRNAAE NGAIQNLIKE EAFLGITDEK TEGQFVDLTG
NRLTYTNWNE GEPNNAGSDE DCVLLLKNGQ WNDVPCSTSH
LAVCEFPI

The amino acid sequence of mature human MBL without the signal sequence (SEQ ID NO:2) is:

ETVTCEDAQK TCPAVIACSS PGINGFPGKD GRDGTKGEKG
EPGQGLRGLQ GPPGKLGPPG NPGPSGSPGP KGQKGDPGKS
PDGDSSLAAS ERKALQTEMA RIKKWLTFSL GKQVGNKFFL
INGEIMTFEK VKALCVKFQA SVATPRNAAE NGAIQNLIKE
EAFLGITDEK TEGQFVDLTG NRLTYTNWNE GEPNNAGSDE
DCVLLLKNGQ WNDVPCSTSH LAVCEFPI

The amino acid sequence of a truncated MBL that retains microbe surface-binding (SEQ ID NO:3) is:

AASERKALQT EMARIKKWLT FSLGKQVGNK FFLINGEIMT
FEKVKALCVK FQASVATPRN AAENGAIQNL IKEEAFLGIT
DEKTEGQFVD LTGNRLTYTN WNEGEPNNAG SDEDCVLLLK
NGQWNDVPCS TSHLAVCEFP I

The amino acid sequence of the carbohydrate recognition domain (CRD) of human MBL (SEQ ID NO:4) is:

VGNKFFLING EIMTFEKVKA LCVKFQASVA TPRNAAENGA
IQNLIKEEAF LGITDEKTEG QFVDLIGNRL TYTNWNEGEP
NNAGSDEDCV LLLKNGQWND VPCSTSHLAV CEFPI

The amino acid sequence of the neck and carbohydrate recognition domain of MBL (SEQ ID NO:5) is:

PDGDSSLAAS ERKALQTEMA RIKKWLTFSL GKQVGNKFFL
TNGEIMTFEK VKALCVKFQA SVATPRNAAE NGAIQNLIKE
EAFLGITDEK TEGQFVDLTG NRLTYTNWNE GEPNNAGSDE
DCVLLLKNGQ WNDVPCSTSH LAVCEFPI

The truncated forms of the naturally-occurring protein include portions of any one of SEQ ID NOs:1-5 lacking 1, 2, 3, 4, 5, 6, 7, 8, 9, 10, 15, 20, 25, 30, 35, 40, 45, 50, 55, 60, 65, 70, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:1-5 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

As to particularly useful truncated forms of the protein, one example is the full-length amino acid sequence of the carbohydrate recognition domain (CRD) of MBL, shown in SEQ ID NO:4. In addition, suitable CRDs include CRDs having an amino acid sequence of about 10 to about 110 amino acid residues, or about 50 to about 100 amino acid residues, of SEQ ID NO:4. In some aspects, the microbe-binding domain can have an amino acid sequence of at least about at least about 10, at least about 15, at least about 20, at least about 30, at least about 40, at least about 50, at least about 60, at least about 70, at least about 80, at least about 90, at least about 100, at least about 110 amino acid residues or more, of SEQ ID NO:4. Accordingly, in some aspects, the carbohydrate recognition domain of an engineered MBL protein can comprise SEQ ID NO:4. In some aspects, the carbohydrate recognition domain of an engineered MBL protein can comprise a fragment of SEQ ID NO:4 as defined above. Also, exemplary amino acid sequences of such fragments include, but are not limited to, ND, EZN (where Z is any amino acid, e.g., P), NEGEPNNAGS (SEQ ID NO:10) or a fragment thereof comprising EPN, GSDEDCVLL or a fragment thereof comprising E, and LLLKNGQWNDVPCST (SEQ ID NO:11) or a fragment thereof comprising ND. Modifications to such CRD fragments, e.g., by conservative substitution (i.e., where an amino acid is replace by an amino acid within the same class of amino acids, where the classes are: aliphatic amino acids (G, A, L, V, I); hydroxyl or sulfur/selenium-containing amino acids (S, C, U, T, M); aromatic amino acids (F, Y, W); basic amino acids (H, K, R); and acidic amino acids (D, E, N, Q)), are also within the scope described herein. In some aspects, the MBL or a fragment thereof used in the microbe surface-binding domain of the engineered MBLs described herein can be a wild-type molecule or a recombinant molecule.

The sequence variants of the naturally-occurring protein and the truncated forms thereof (e.g. SEQ ID NOs:1-5) include proteins having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:1-5, or truncated forms thereof, that retain the ability to reduce platelet activation in blood of the protein upon which they are based.

### Engineered MTMs Comprising MBL

In other particular aspects and embodiments of the invention, the MTM is an engineered MTM comprising MBL, as defined above, and a second protein in the form of a fusion protein. An exemplary fusion protein comprises some or all of naturally-occurring MBL, such as the carbohydrate recognition domain (CRD) of MBL, and a portion of an immunoglobulin, such as the Fc domain. In use, the Fc domain dimerizes and strengthens the avidity and affinity of the binding by MBLs to monomeric sugars. In some aspects, the N-terminus of fusion proteins can further comprise an oligopeptide linker adapted to bind a solid substrate and orient the CRD of the MBL domain away from a substrate to which it is immobilized. As discussed above, engineered forms of MBLs are known in the art and include each of the forms of MBL disclosed in U.S. Pat. No. 9,150,631, U.S. Patent Pub. 2016/0311877, U.S. Patent Pub. 2019/0077850, U.S. Pat. Nos. 9,593,160, 10,435,457, U.S. Patent Pub. 2015/0173883 and International Application Publication No. WO 2011/090954, the entire disclosures of which are hereby incorporated by reference in their entirety.

FcMBL is a specific engineered form of MBL of the invention that comprises the neck and CRD domains of MBL linked to an IgG Fc domain. Proline 81 of mature MBL (SEQ ID NO:2) is a convenient N-terminal point at which to begin the sequence of this engineered construct. For example, the neck and CRD domains (SEQ ID NO:5) of MBL are fused downstream (C-terminal) to the Fc domain of human IgG (Fcγ). The Fc domain may include the CH2-CH3 interface of the IgG Fc domain, which contains the binding sites for a number of Fc receptors including Staphylococcal protein A. In use, the Fc domain dimerizes and strengthens the avidity and affinity of the binding by MBLs to monomeric sugars. FcMBL is described in detail in U.S. Pat. No. 9,150,631, the entire disclosure of which is hereby incorporated by reference in its entirety.

Specific examples of FcMBLs that may be used in each of the aspects and embodiments of the invention include, but are not limited to, proteins where the neck and CRD domains of MBL are linked to an Fc component of human IgG1, with examples of the resulting constructs set forth in SEQ ID NOs:6, 7 and 9, and proteins where the CRD domain alone of MBL is linked to an Fc component of human IgG1, with an example of the resulting construct set forth in SEQ ID NO:8.

FcMBL.81 (SEQ ID NO: 6):
EPKSSDKTHT CPPCPAPELL GGPSVELFPP KPKDTLMISR TPEVTCVVVD
VSHEDPEVKF NWYVDGVEVH NAKTKPREEQ YNSTYRVVSV LTVLHQDWLN
GKEYKCKVSN KALPAPIEKT ISKAKGQPRE PQVYTLPPSR DELTKNQVSL
TCLVKGFYPS DIAVEWESNG QPENNYKTTP PVLDSDGSFF LYSKLTVDKS
RWQQGNVFSC SVMHEALHNH YTQKSLSLSP GAPDGDSSLA ASERKALQTE
MARIKKWLTF SLGKQVGNKF FLINGEINIT FEKVKALCVK FQASVATPRN
AAENGAIQNL IKEEAFLGIT DEKTEGQFVD LTGNRLTYTN WNEGEPNNAG
SDEDCVLLLK NGQWNDVPCS TSHLAVCEFP I
AKT-FcMBL (SEQ ID NO: 7):
AKTEPKSSDK THTCPPCPAP ELLGGPSVFL FPPKPKDTLM ISRTPEVTCV
VVDVSHEDPE VKFNWYVDGV EVHNAKTKPR EEQYNSTYRV VSVLTVLHQD
WLNGKEYKCK VSNKALPAPI EKTISKAKGQ PREPQVYTLP PSRDELTKNQ
VSLTCLVKGF YPSDIAVEWE SNGQPENNYK TTPPVLDSDG SFFLYSKLTV
DKSRWQQGNV FSCSVMHEAL HNHYTQKSLS LSPGAPDGDS SLAASERKAL
QTEMARIKKW LTFSLGKQVG NKFFLINGEI MTFEKVKALC VKFQASVATP
RNAAENGAIQ NLIKEEAFLG ITDEKTEGQF VDLIGNRLTY TNWNEGEPNN
AGSDEDCVLL LKNGQWNDVP CSTSHLAVCE FPI
FcMBL.111 (SEQ ID NO: 8):
EPKSSDKTHT CPPCPAPELL GGPSVFLFPP KPKDTLMISR TPEVTCVVVD
VSHEDPEVKF NWYVDGVEVH NAKTKPREEQ YNSTYRVVSV LTVLHQDWLN
GKEYKCKVSN KALPAPIEKT ISKAKGQPRE PQVYTLPPSR DELTKNQVSL
TCLVKGFYPS DIAVEWESNG QPENNYKTTP PVLDSDGSFF LYSKLTVDKS
RWQQGNVFSC SVMHEALHNH YTQKSLSLSP GATSKQVGNK FFLINGEEVI
TFEKVKALCV KFQASVATPR NAAENGAIQN LIKEEAFLGI TDEKTEGQFV
DLTGNRLTYT NWNEGEPNNA GSDEDCVLLL KNGQWNDVPC STSHLAVCEF
PI
FcMBL (SEQ ID NO: 9):
AKTEPKSSDK THTCPPCPAP ELLGGPSVFL FPPKPKDTLM ISRTPEVTCV
VVDVSHEDPE VKENWYVDGV EVHNAKTKPR EEQYDSTYRV VSVLIVLHQD
WINGKEYKCK VSNKALPAPI EKTISKAKGQ PREPQVYTLP PSRDELTKNQ
VSLTCLVKGF YPSDIAVEWE SNGQPENNYK TTPPVLDSDG SFFLYSKLTV
DKSRWQQGNV FSCSVMHEAL HNHYTQKSLS LSPGAPDGDS SLAASERKAL
QTEMARIKKW LTESLGKOVG NKFFLINGEI MTFEKVKALC VKFQASVATP
RNAAENGAIQ NLIKEEAFLG ITDEKTEGQF VDLTGNRLTY TNWNEGEPNN
AGSDEDCVLL LKNGQWNDVP CSTSHLAVCE FPI

In SEQ ID NO:9, the residues with a single underscore correspond to the Fc portion, the residues with a double underscore correspond to the MBL neck, and those residues without underscore correspond to the MBL carbohydrate-binding domain.

Various genetically engineered versions of the MTM (e.g., FcMBL) are described in International Application Pub. Nos. WO 2011/090954 and WO 2013/012924, as well as U.S. Pat. Nos. 9,150,631 and 9,593,160, the contents of each of which are incorporated herein by reference in their entireties. Lectins and other mannose binding molecules are also described in, for example, U.S. Pat. Nos. 9,150,631 and 9,632,085, and PCT application publication nos. WO/2011/090954, WO2013012924 and WO/2013/130875, the contents of all of which are incorporated herein by reference in their entireties.

In aspects where the MTM is FcMBL, the Fc region or a fragment thereof can comprise at least one mutation, e.g., to modify the performance of the engineered MBL. For example, in some aspects, the half-life of the engineered MBL described herein can be increased, e.g., by mutating the lysine (K) at the residue 232 to alanine (A) as shown in the Fc domain sequence provided in SEQ ID NO:12. Other mutations, e.g., located at the interface between the CH2 and CH3 domains shown in Hinton et al (2004) J Biol Chem. 279:6213-6216 and Vaccaro C. et al. (2005) Nat Biotechnol. 23: 1283-1288, can be also used to increase the half-life of the IgG1 and thus the engineered MBL.

SEQ ID NO: 12:
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GA

In some aspects and embodiments, the FcMBL of the invention comprises or consists of an amino acid sequence having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:6-9, that retain the active of protein upon which they are based.

The exemplary MBL sequences provided herein are not construed to be limiting. For example, while the exemplary sequences provided herein are derived from a human species, amino acid sequences of the same carbohydrate recognition domain in plants and other animal species such as mice, rats, porcine, bovine, feline, and canine are known in the art and within the scope described herein.

### Other Collectins

In addition to the aspects and embodiments of the invention defined above that comprise the collectin MBL (whether naturally-occurring or engineered), the present invention encompasses use of any other collectin in an MTM that binds MAMPs, when used in conjunction with the methods and compositions of the invention. Thus, any of the following additional collectins may also be used in the aspects and embodiments of the invention as defined herein: (i) surfactant protein A (SP-A; SEQ ID NO:13), (ii) surfactant protein D (SP-D; SEQ ID NO:14), (iii) collectin liver 1 (CL-L1; SEQ ID NO:15), (iv) collectin placenta 1 (CL-P1; SEQ ID NO:16), (v) conglutinin collectin of 43 kDa (CL-43; SEQ ID NO:17), (vi) collectin of 46 kDa (CL-46; SEQ ID NO:18), (vii) collectin kidney 1 (CL-K1; SEQ ID NO:19), and (viii) conglutinin (SEQ ID NO:20).

As with MBL, both naturally-occurring collectins and engineered forms of the proteins may be used in the invention. Engineered forms of the proteins include, but are not limited to, truncated forms of the naturally-occurring proteins, sequence variants of the naturally-occurring proteins, sequence variants of the truncated forms of the proteins, fusion proteins comprising the naturally-occurring protein, fusion proteins comprising the truncated forms of the proteins, and fusion proteins comprising the sequence variants.

The truncated forms of the naturally-occurring collectins include portions of any one of SEQ ID NOs:13-20 lacking 1, 2, 3, 4, 5, 6, 7, 8, 9, 10, 15, 20, 25, 30, 35, 40, 45, 50, 55, 60, 65, 70, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:13-20 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

The sequence variants of the naturally-occurring protein and the truncated forms thereof (e.g. SEQ ID NOs:13-20) include proteins having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:13-20, or truncated forms thereof, that retain the activity of the protein upon which they are based.

### Labels

The MTMs of the present invention may be labeled to allow them to be detected after binding to microbes or microbial components from a sample. The identity of the detectable label is limited only in that it can be discerned by the human eye or via a detector in the context of the detection device. Suitable detectable labels include colored or fluorescent particles, such a Europium particles or colloidal gold. Other acceptable labels include latex, which may itself be tagged with colored or fluorescent dyes, and magnetic or paramagnetic components. A further detectable label is a plasmonic fluor, wherein instead of assaying for a color change, one detects fluorescence. Ultrabright fluorescent nanolabels can also be used to improve the limit of detection in the detection devices of the invention, compared with conventional fluorophores.

Other detectable labels include, but are not limited to, an enzyme (e.g., peroxidase, alkaline phosphatase, glucose oxidase), a metal (e.g., gold for electron microscopy applications), a fluorescent marker (e.g., for immunofluorescence and flow cytometry applications, including CYE dyes, fluorescein isothiocyanate, rhodamine, phycoerytherin, phycocyanin, allophycocyanin, o-phthaldehyde and fluorescamine), a fluorescence-emitting metals (e.g., ¹⁵²Eu), a radioactive marker (e.g., radioisotopes for diagnostic purposes, including ³H, ¹³¹I, ³⁵S, ¹⁴C, and ¹²⁵I), a chemiluminescent marker (e.g., luminol, luciferin, isoluminol, theromatic acridinium ester, imidazole, acridinium salt and oxalate ester), and a protein tag (e.g., biotin, phycobiliprotein, c-Myc, HA, VSV-G, HSV, FLAG, V5, or HIS).

### Compositions Comprising MTMs

The invention also includes compositions comprising one or more of types of MTMs defined herein, i.e. both naturally-occurring MTMs and engineered MTMs. As indicated above, particular MTMs can be defined based on (i) structural terms (e.g. based on the components of the MTM; the amino acid sequence of the MTM; the nucleic acid sequence of the MTM; etc.), (ii) functional terms (e.g. the identity of the MAMP bound by the PRR portion of the microbe-binding domain; the affinity or avidity of binding to the MAMP; etc.), or (iii) both structural and functional terms. When a composition is defined as comprising two or more types of MTMs, it should be understood that “types” of MTMs in the composition differ based on structural and/or functional terms from each other. When there is more than one type of MTM in a composition, the composition is said to comprise a mixture of different types of MTMs within the composition.

An advantage of the present invention is the composition can be customized based on the desired use, e.g., diagnosis and/or therapy and/or filtration. As a non-limiting example, the composition can be selected based on the organisms endemic to a particular area.

The compositions may comprise different types of MTMs within one category of MTMs, as defined herein, or the compositions may comprise different types of MTMs within two or more different categories of MTMs, as defined herein. Thus, the compositions of the invention include “cocktails” of different types of MTMs, wherein the composition can include 1, 2, 3, 4, 5, 6, 7, 8, 9, 10 or more different types of MTMs within a single composition.

The compositions of the invention may comprise mixtures of naturally-occurring MTMs (e.g. MBLs), mixture of both naturally-occurring MTMs (e.g. MBLs) and the engineered MTMs defined herein (e.g. FcMBLs), or mixtures of only engineered MTMs (e.g., FcMBLs).

Depending on the manner in which the MTMs are used, the compositions comprising one or more different type of MTM may include suitable carriers and diluents. Suitable carriers and diluents are commonly known and will vary depending on the MTM being used and the mode of use. Examples of suitable carriers and diluents include water, buffered water, saline, buffered saline, dextrose, glycerol, ethanol, and combinations thereof, propylene glycol, polysorbate 80 (Tween-80™), poly(ethylene)glycol 300 and 400 (PEG 300 and 400), hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes, other stabilizing agents, solubilizing agents and surfactants, buffers and preservatives, tonicity agents, bulking agents, and lubricating agents

The compositions of the invention may also comprise one or more therapeutic agents, such as an antimicrobial agent. An MTM binds to one or more microbes or microbe components. An antimicrobial agent can optionally be included to treat (e.g. kill or inactivate) one or more known or suspected pathogens.

When the compositions comprise one or more antimicrobial agents, suitable agents include, but are not limited to, antibiotics, antivirals and antifungals. Antibiotics can be from classes including but not limited to Cephalosporin, Glycopeptide, Cyclic lipopeptide, Aminoglycoside, Macrolide, Oxazolidinone, Fluoroquinolones, Lincosamides, or Carbapenem. Antifungals can be from classes including but not limited to Polyenes, Azoles, Nucleoside Analog, Echinocandin, or Allylamine. Antivirals can be from classes including but not limited to CCR5 anatonists, Fusion inhibitors, Nucleoside/Nucleotide reverse transcriptase inhibitors (NRTIs), Non-nucleoside reverse transcriptase inhibitors (NNRTIs), Nucleotide reverse transcriptase inhibitors (NtRTIs), Integrase inhibitors, Protease inhibitors, DNA polymerase inhibitors, Guanosine analogs, Interferon-alpha, M2 ion channel blockers, Nucleoside inhibitors, NS5A polymerase inhibitors, NS3/4A protease inhibitors, Neuraminidase inhibitors, Nucleoside analogs, and Direct acting antivirals (DAAs). Examples of antimicrobials include but are not limited to aminoglycosides, ansamycins, beta-lactams, bis-biguanides, carbacephems, carbapenems, cationic polypeptides, cephalosporins, fluoroquinolones, glycopeptides, iron-sequestering glycoproteins, linosamides, lipopeptides, macrolides, monobactams, nitrofurans, oxazolidinones, penicillins, polypeptides, quaternary ammonium compounds, quinolones, silver compounds, sulfonamides, tetracyclines, and any combinations thereof.

Some exemplary antibiotics that may be included in the compositions of the invention include, but are not limited to, broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefmetazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin IV Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

Some exemplary antifungals that may be included in the compositions of the invention include, but are not limited to, polyene antifungals, Amphotericin B, Candicidin, Filipin, Hamycin, Natamycin, Nystatin, Rimocidin, imidazole antifungals, triazole antifungals, thiazole antifungals, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Triazoles[edit], Albaconazole, Efinaconazole, Epoxiconazole, Fluconazole, Isavuconazole, Itraconazole, Posaconazole, Propiconazole, Ravuconazole, Terconazole, Voriconazole, Abafungin, Allylamines, amorolfin, butenafine, naftifine, terbinafine, Echinocandins, Anidulafungin, Caspofungin, Micafungin, Aurones, Benzoic acid, Ciclopirox, Flucytosine, 5-fluorocytosin, Griseofulvin, Haloprogin, Tolnaftate, Undecylenic acid, Triacetin, Crystal violet, Castellani's paint, Orotomide, Miltefosine, Potassium iodide, Coal tar, Copper(II) sulfate, Selenium disulfide, Sodium thiosulfate, Piroctone olamine, Iodoquinol, clioquinol, Acrisorcin, Zinc pyrithione, and Sulfur. Additional antifungals known in the art can also be used.

Some exemplary antivirals that may be included in the compositions of the invention include, but are not limited to, Abacavir, Acyclovir, Adefovir, Amantadine, Ampligen, Amprenavir, antiretroviral, Arbidol, Atazanavir, Atripla, Boceprevir, Cidofovir, Combivir, Daclatasvir, Darunavir, Delavirdine, Dasabuvir, Didanosine, Docosanol, Dolutegravir, Doravirine, Ecoliever, Edoxudine, Efavirenz, Elbasvir, Emtricitabine, Enfuvirtide, Entecavir, Etravirine, Famciclovir, Fomivirsen, Fosamprenavir, Foscarnet, Fosfonet, Fusion inhibitor, Ganciclovir, Gemcitabine, Glecaprevir, Grazoprevir, Ibacitabine, Idoxuridine, Imiquimod, Imunovir, Indinavir, Inosine, Integrase inhibitor, Interferon, Interferon type I, Interferon type II, Interferon type III, Lamivudine, Ledipasvir, Lopinavir, Lopiravir, Loviride, Maraviroc, Methisazone, Moroxydine, Nelfinavir, Nevirapine, Nexavir, Nitazoxanide, Norvir, Nucleoside analogues, Ombitasvir, Oseltamivir (Tamiflu), Paritaprevir, Peglyated Interferon-alpha, Peginterferon alfa-2a, Penciclovir, Peramivir, Pibrentasvir, Pleconaril, Podophyllotoxin, Protease inhibitor, Pyramidine, Raltegravir, Reverse transcriptase inhibitor, Ribavirin, Rilpivirine, Rimantadine, Ritonavir, Saquinavir, Simeprevir, Sofosbuvir, Stavudine, Synergistic enhancer (antiretroviral), Telaprevir, Telbivudine, Tenofovir, Tenofovir disoproxil, Tipranavir, Trifluridine, Trizivir, Tromantadine, Truvada, Valaciclovir (Valtrex), Valganciclovir, Velpatasvir, Vicriviroc, Vidarabine, Viramidine, Voxilaprevir, Zalcitabine, Zanamivir (Relenza), Zidovudine. Additional antivirals known in the art can also be used.

The compositions of the invention can take many different forms, varying widely based on (i) the identity of the MTMs in the composition, (ii) the identity of other components in the composition, and (iii) the intended use of the composition, to name only a few of the relevant factors.

For pharmaceutical uses, the compositions of the invention may further comprise pharmaceutically acceptable carriers and diluents when administered to or used on a living subject. Suitable carriers and diluents are commonly known and will vary depending on the MTM being used or administered and the mode of use or administration. Examples of suitable carriers and diluents include saline, buffered saline, dextrose, water-for-injection, glycerol, ethanol, and combinations thereof, propylene glycol, polysorbate 80 (Tween-80™), poly(ethylene)glycol 300 and 400 (PEG 300 and 400), PEGylated castor oil (e.g. Cremophor EL), poloxamer 407 and 188, a cyclodextrin or a cyclodextrin derivative (including HPCD ((2-hydroxypropyl)-cyclodextrin) and (2-hydroxyethyl)-cyclodextrin), hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes, other stabilizing agents, solubilizing agents and surfactants, buffers and preservatives, tonicity agents, bulking agents, and lubricating agents. The formulations comprising MTMs will typically have been prepared using MTMs proteins from cultures prepared in the absence of any non-human components, such as animal serum (e.g., bovine serum albumin).

As described above, a composition of the present invention can comprise any suitable formulation, including but not limited to an aerosol, a rinse, a spray, a cream, a powder, or an ointment that can be administered nasally, orally, and/or ocularly.

In some aspects, a composition of the present invention comprises an aerosolized composition. The composition can comprise a solution, e.g., a liquid, of MTMs that can be aerosolized by any aerosol generating/delivery device as known in the art (described below). The aerosolized particles can be of a suitable size for deposition into the lungs, including the smaller airways and alveoli, or onto a carrier or inert surface. An exemplary sized particle can range from about 1 micron to about 5 microns, for example, about 1 micron, or about 2 microns, or about 3 microns, or about 4 microns, or about 5 microns.

An aerosolized composition can be delivered to a subject, e.g., via the nose, eyes or mouth, by any aerosol generating/delivery device as known in the art. In some aspects, the delivery device comprises a nebulizer where the nebulizer can comprise a small volume nebulizer, a large volume nebulizer, or an ultrasound nebulizer. In some aspects, the delivery device comprises a metered-dose inhaler. In these aspects, the inhaler can include spacers or holding chambers. In some aspects, the delivery device comprises a dry-powder inhaler.

The aerosolized composition can be administered to a subject preventatively to prevent one or more pathogens from entering a subject, e.g., into the eye(s), nose, mouth, and/or respiratory system including the airway, lungs and blood vessels, and blood. The MTMs of the composition act by binding to one or more microbes or microbe components and preventing the one or more microbe or microbe components, thus immobilizing it and preventing it from entering the eye(s), nose, mouth, and/or respiratory system including the airway, lungs and blood vessels, and ultimately the blood. Aerosol administration enables a direct, topical application of the composition to the target site, such as the lung, which may be particularly advantageous in preventing a respiratory-transmitted infection such as SARS-CoV-2.

In some aspects, a composition of the present invention comprises an ocular rinse or ointment. The composition can be mixed with any suitable biocompatible ocular rinses that are known in the art. The composition can be administered to a subject by dropping the rinse into the eye, or applying an ointment proximate to the eye, e.g., the eye-lids.

In some aspects, a composition of the present invention comprises a mouth rinse. The composition can be mixed with any suitable biocompatible mouth rinses that are known in the art. The composition can be administered to a subject's mouth by swooshing, gargling and/or swallowing the rinse.

In some aspects, a composition of the present invention comprises a nasal rinse, spray or ointment. The composition can be mixed with any suitable biocompatible nasal rinses or spray that are known in the art. The composition can be administered to a subject by dropping or spraying into the nose, or applying an ointment in or proximate to the nose, e.g., the nostrils, to prevent pathogens from binding to the nasal mucosa. For example, the compositions of the present invention can be used to block binding of SARS-CoV-2 to ACE2 receptors in the nasal cavity.

Any of the MTM compositions described herein can be administered to a subject after exposure to a pathogen, for example, to decrease the pathogen load on the subject and/or to treat a subject having one or more infections. In some aspects, if a subject is exposed to a known pathogen, a particular antimicrobial, based on the pathogen, can be selected and added to the composition.

In some aspects, MTM compositions (e.g., engineered MTMs as described therein), methods, systems, and assays are further described in at least one of the following: U.S. provisional application Nos. 61/296,222, 61/508,957, 61/604,878, 61/605,052, 61/605,081, 61/788,570, 61/846,438, 61/866,843, 61/917,705, 62/201,745, 62/336,940, 62/543,614; PCT application numbers PCT/US2011/021603, PCT/US2012/047201, PCT/US2013/028409, PCT/US2014/028683, PCT/US2014/046716, PCT/US2014/071293, PCT/US2016/045509, PCT/US2017/032928; U.S. patent application Ser. Nos. 13/574,191, 14/233,553, 14/382,043, 14/766,575, 14/831,480, 14/904,583, 15/105,298, 15/415,352, 15/483,216, 15/668,794, 15/839,352, 16/059,799, 16/302,023, 16/553,635; and U.S. Pat. Nos. 9,150,631, 9,593,160, 9,632,085, 9,791,440, and 10,435,457; the contents of each of which are incorporated by reference herein in their entireties.

### Polynucleotides

The invention includes polynucleotides comprising nucleotide sequences encoding each of the MTMs provided herein, as well as complementary strands thereof.

These polynucleotides include those encoding full-length human MBL (SEQ ID NO:1), mature human MBL without the signal sequence (e.g. SEQ ID NO:2), a truncated human MBL that retains microbe surface-binding (e.g. SEQ ID NO:3), the carbohydrate recognition domain (CRD) of human MBL (e.g. SEQ ID NO:4), and the neck and carbohydrate recognition domain of human MBL (e.g. SEQ ID NO:5), and sequence variants thereof having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any of SEQ ID NOs:1-5.

These polynucleotides also include those encoding the FcMBLs of SEQ ID NOs: 6, 7, 8 and 9, and sequence variants thereof having at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any of SEQ ID NOs: 6, 7, 8 and 9.

The invention also includes cloning and expression vectors comprising the polynucleotides, and host cells comprising either the polynucleotides or the expression vectors. Such host cells may be mammalian or non-mammalian cells, including, but not limited to, E. coli, and insect cells. The invention further includes methods of producing the MTMs defined herein, comprising culturing the host cells under conditions promoting expression of the MTMs encoded by the polynucleotides and expression vectors, and recovering the MTMs from the cells or cell cultures.

### Cells

Cell lines may be used to express the MTMs of the present invention. Suitable cell lines are limited only in that they can stably express the MTMs. Suitable cell lines include, but are not limited to, lymphocytes and non-lymphoid cells, including T cells and neutrophils.

The invention thus includes cells that stably express one or more of the MTMs defined herein on their surface. In some instances, these cells are termed “biosensor cells” herein. In particular embodiments, the invention includes biosensor cells stably expressing on their surface more or more of the MTMs defined herein. Suitable cell lines include, but are not limited to, lymphocytes and non-lymphoid cells, including T cells and neutrophils.

### Side Groups

The MTMs of the invention can be engineered to display side groups that augment, enhance or otherwise alter selected characteristics of the MTMs. For example, the MTMs of the invention may be engineered to display polyfluoro groups on any portion of the molecule. Such groups include fluoropolymers comprising terminal polyfluoro-oligomeric groups. These groups can aid in reducing thrombosis that may result, for example, when blood comes into contact with non-self surfaces. Coating of such surfaces with MTMs displaying polyfluoro-groups can reduce coagulation.

### Diagnostic Devices

As suggested above, the MTMs and compositions of the invention can be used in diagnostic devices (and related methods) to detect and/or identify microbes and microbial components in a sample.

The diagnostic devices of the present invention are limited only in that (i) they are devices (or components thereof) that may be used in the diagnosis of an infection, disease or some other condition in a subject, and (ii) they contain one or more MTMs of the invention. In a typical example, the diagnostic devices or at least a component thereof will be coated with MTMs or otherwise display MTMs on a surface of the device or component thereof.

The diagnostic devices of the invention include, but are not limited to, the following: dipsticks, test strips, and any other sample collection devices known in the art. At least one surface of the device is coated with MTMs of the invention, or otherwise display MTMs such that the MTMs are exposed to a sample under conditions permitting binding of microbes or microbial components in the sample by the MTMs.

Alternatively, or in addition, the diagnostic devices of the invention comprise at least one component that is coated with MTMs of the invention, or otherwise display MTMs such that the MTMs are exposed to a sample under conditions permitting binding of microbes or microbial components in the sample by the MTMs. Such components include, but are not limited to, supports (e.g. graphene), beads (e.g. gold particles), particles (including nanoparticles, microparticles, polymer microbeads, magnetic microbeads, and the like), filters, fibers, screens, mesh, cartridges, tubes, hollow fibers, scaffolds, plates, channels, magnetic materials, medical apparatuses (e.g., needles or catheters) or implants, filtration devices or membranes, cartridges (e.g. hollow fiber cartridges), microfluidic devices, mixing elements (e.g., spiral mixers), and other substrates commonly utilized in assay formats, and any combinations thereof.

In some aspects, the support is a magnetic support. In some aspects, the magnetic support is a superparamagnetic support. In some aspects, the magnetic support comprises a magnetic bead, a superparamagnetic bead, or a magnetic microbead. In some aspects, the support is a gold, silver, or graphene, for example, for use in surface plasmon resonance (SPR) and localized surface plasmon resonance (LSPR).

The diagnostic devices of the invention may be used in a wide variety of diagnostic applications including, but not limited to, methods of detecting the presence of a microbe or microbial component in a bodily fluid of a subject. Such methods include contacting a bodily fluid of the subject with a diagnostic device of the invention under conditions that permit binding of microbes or microbial components by MTMs displayed by the diagnostic device, thus detecting microbes or microbial components in the bodily fluid of the subject. In one aspect, the microbe is a bacteria. In another aspect, the microbe is a virus. In further aspect, the microbe is a fungus. In further aspect, the microbe is a protozoan. Optionally, such methods can include one or more of the following additional steps: (i) quantifying the amount of microbe or microbial component in the bodily fluid; (ii) identifying the microbe in the bodily fluid. When the MTM used in conjunction with the diagnostic device is a species-specific MTM, for example, the microbe being detected by the diagnostic device can be identified to the taxonomic level of species. However, when the MTM(s) used in conjunction with the diagnostic device are not species-specific, i.e. the MTM(s) recognize and bind a family or genus of microbes and cannot identify the microbe to the taxonomic level of species, a further identification means may be used to identify the microbe to the selected taxonomic level.

With specific references to diagnostic devices, the MTM may be linked to an ELISA plate. Use of an ELISA plate can allow for multiplexing of samples. Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries. Different forms of ELISA are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In some aspects, the MTM is coated and/or immobilized on the solid phase of multi-well plate, i.e., conjugated to a solid surface (usually a polystyrene micro titer plate, e.g., an “ELISA plate”). Immobilization can be either non-specific (e.g., by adsorption to the surface) or specific (e.g. where another molecule immobilized on the surface is used to capture the microbe-targeting molecule).

After the MTM is immobilized, the sample is added, forming a complex with the MTM. Between each step the plate is typically washed with a mild detergent solution to remove any molecules that are not specifically bound. After the final wash step, the plate is prepared for detection by a mass spectrometric method, as described herein. Such preparation can include but is not limited to eluting the microbe or microbe components, contacting the microbe or microbe components with a protease, contacting the microbe or microbe components with a solution that is more acidic than the microbe or microbe components, and/or contacting the microbe or microbe components with a matrix or matrix solution. Additional methods of preparing the isolated microbe or microbe components for detection by a mass spectrometric method can be performed as described herein.

In some aspects, described herein are methods of isolating microbes or microbe components using a diagnostic device. In some aspects, the step of isolating comprises applying a magnet to the sample, for example to capture an engineered MTM linked to a magnetic support, i.e. the diagnostic device. In some aspects, the use of magnetic microparticles (e.g., superparamagnetic microparticles) can allow for easier washing and recovery of the microparticles, for automating the time of incubation with the sample, and also for working with whole blood with no interference from the erythrocytes. In some aspects, the magnet can be any magnetic material capable, a handheld magnet, a magnet formatted to a plate design such as a multi-well magnetic separator, a neodymium magnet tube rack, an automated magnet, and the like.

In some aspects, the methods described herein comprise contacting a sample with an MTM linked to a support (the diagnostic device) and isolating the microbe or microbe components bound to the MTM. The support can be any support as described herein, including but not limited to beads or particles (including nanoparticles, microparticles, polymer microbeads, magnetic microbeads, and the like), filters, fibers, screens, mesh, tubes, hollow fibers, scaffolds, plates, channels, gold particles, magnetic materials, medical apparatuses (e.g., needles or catheters) or implants, dipsticks or test strips, filtration devices or membranes, hollow fiber cartridges, microfluidic devices, mixing elements (e.g., spiral mixers), extracorporeal devices, and other substrates commonly utilized in assay formats, and any combinations thereof.

In some aspects, the step of isolating comprises washing the support with a buffer to remove unbound cells or biomolecules. The buffer can be any buffer as described herein, including but not limited to tris-buffered-saline, phosphate buffer saline, water, HPLC grade H₂O, comprising octyl-β-D-glucopyranoside and/or calcium (TBSG Ca²⁺). In some aspects, the step of washing can be performed at least 1, at least 2, at least 3, at least 4, or at least 5 times.

In some aspects, the step of isolating further comprises eluting the microbe or microbe components from the support (diagnostic device) as described herein.

While, in some aspects, microbes and/microbial matter (e.g., MAMPs) can be captured by MTM-coated solid substrates (i.e., diagnostic devices) prior to detection, in other aspects, microbes and/or microbial matter (e.g., MAMPs) can be detected by MTM-coated detectable label as defined herein, e.g., MTM-coated fluorescent molecule, without prior capture. In these aspects, the microbes and/or microbial matter (e.g., MAMPs) can be bound, mounted or blotted onto a solid surface, e.g., a tissue surface, and a membrane surface (i.e., a diagnostic device).

The microbes and/or microbial matter (e.g., MAMPs) bound to MTM-coated (e.g., lectin-coated) solid substrates (e.g., polymeric or magnetic particles or beads) or a solid surface can be detected by any methods known in the art or as described herein.

### MTM-Coated Surfaces

In each of the devices of the invention, whether diagnostic, therapeutic or filtration, the MTMs may be coated and/or immobilized onto at least one surface of the device, or a component of the device, such as the MTMs coat all surfaces of the device or component, or only selected portions of the device or component.

Immobilization (via coating) of MTMs onto a surface can be either non-specific (e.g., by adsorption to the surface) or specific (e.g. where another molecule, such as a linker, immobilized on the surface is used to capture the MTM). MTMs may be linked to the surface through one or more linkers which may be cleavable to accommodate release or elution of the bound target molecules for subsequent analysis. Binding domains may be calcium-dependent and systems and methods of the invention may include the addition of calcium to promote target binding. MTMs may attach to the one or more surfaces though a covalent linking process. Substrate linkage may be accomplished through, for example, biotin-avidin binding, 1-Ethyl-3-[3-dimethylaminopropyl]carbodiimide hydrochloride (EDC or EDAC), hydroxybenzotriazole (HOBT), N-Hydroxysuccinimide (NHS), 2-(1H-7-Azabenzotriazol-1-yl)-1,1,3,3-tetramethyl uronium hexafluorophosphate methanaminium (HATU), silanization, surface activation through plasma treatment, and the like.

In some embodiments, the surface is fabricated or coated with a material prior to being coated by MTMs, where the material is one or more of polydimethylsiloxane, polyimide, polyethylene terephthalate, polymethylmethacrylate, polyurethane, polyvinylchloride, polystyrene polysulfone, polycarbonate, polymethylpentene, polypropylene, a polyvinylidine fluoride, polysilicon, polytetrafluoroethylene, polysulfone, acrylonitrile butadiene styrene, polyacrylonitrile, polybutadiene, poly(butylene terephthalate), poly(ether sulfone), poly(ether ether ketones), poly(ethylene glycol), styrene-acrylonitrile resin, poly(trimethylene terephthalate), polyvinyl butyral, polyvinylidenedifluoride, poly(vinyl pyrrolidone), and any combination thereof.

In some embodiments, MTMs can be conjugated to the surface by methods well known in the art for conjugating peptides with other molecules. For example, Hermanson, BIOCONJUGATE TECHNIQUES (2nd Ed., Academic Press (2008)) and Niemeyr, Bioconjugation Protocols: Strategies & Methods, in METHODS IN MOLECULAR BIOLOGY (Humana Press, 2004), provide a number of methods and techniques for conjugating peptides to other molecules. de Graaf, et al., 20 Biocojugate Chem. 1281 (2009), provides a review of site-specific introduction of non-natural amino acids into peptides for conjugation.

Alternatively, the surface can be functionalized to include binding molecules that bind selectively with the MTMs. The binding molecule can be bound covalently or non-covalently on the surface. As used herein, the term “binding molecule” refers to any molecule that is capable of specifically binding MTMs, as defined herein. Representative examples of binding molecule include, but are not limited to, antibodies, antigens, lectins, proteins, peptides, nucleic acids (DNA, RNA, PNA and nucleic acids that are mixtures thereof or that include nucleotide derivatives or analogs); receptor molecules, such as the insulin receptor; ligands for receptors (e.g., insulin for the insulin receptor); and biological, chemical or other molecules that have affinity for another molecule, such as biotin and avidin. The binding molecules need not comprise an entire naturally occurring molecule but may consist of only a portion, fragment or subunit of a naturally or non-naturally occurring molecule, as for example the Fab fragment of an antibody. The binding molecule may further comprise a marker that can be detected.

The binding molecule can be conjugated to the surface using any of a variety of methods known to those of skill in the art. The binding molecule can be coupled or conjugated to surface of the substrate covalently or non-covalently. Covalent immobilization may be accomplished through, for example, silane coupling. See, e.g., Weetall, 15 Adv. Mol. Cell Bio. 161 (2008); Weetall, 44 Meths. Enzymol. 134 (1976). The covalent linkage between the binding molecule and the surface can also be mediated by a linker. The non-covalent linkage between the binding molecule and the surface can be based on ionic interactions, van der Waals interactions, dipole-dipole interactions, hydrogen bonds, electrostatic interactions, and/or shape recognition interactions.

As used herein, the term “linker” means a molecular moiety that connects two parts of a composition. Peptide linkers may affect folding of a given fusion protein, and may also react/bind with other proteins, and these properties can be screened for by known techniques. Example linkers, in addition to those described herein, include is a string of histidine residues, e.g., His6; sequences made up of Ala and Pro, varying the number of Ala-Pro pairs to modulate the flexibility of the linker; and sequences made up of charged amino acid residues e.g., mixing Glu and Lys. Flexibility can be controlled by the types and numbers of residues in the linker. See, e.g., Perham, 30 Biochem. 8501 (1991); Wriggers et al., 80 Biopolymers 736 (2005). Chemical linkers may comprise a direct bond or an atom such as oxygen or sulfur, a unit such as NH, C(O), C(O)NH, SO, SO₂, SO₂NH, or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclyl, substituted or unsubstituted C₃-C₁₂ cycloalkyl, where one or more methylenes can be interrupted or terminated by O, S, S(O), SO₂, NH, or C(O).

### Sample Preparation Prior to Diagnosis

Preparing a sample for detecting a microbe or microbe component can include isolating from a sample a microbe or microbe components bound to an MTM on a substrate; digesting the isolated microbe or microbe components with a substance; and contacting the microbe or microbe components with a matrix or matrix solution on a target substrate. In some aspects, the target substrate is evenly sprayed with matrix solution prior to analyzing microbe or microbe components to generate a homogenous layer of crystallized matrix on top of the substrate.

Microbe isolation and analysis may be performed on whole, intact microbes or any portion or subpart thereof (e.g., cell wall components, outer membranes, nucleic acid (e.g., DNA, including 16S ribosomal DNA, and RNA), plasma membranes, ribosomes, microbial capsule, pili, or flagella. Microbe isolation and analysis as described herein may also involve identification of microbe-associated molecular patterns (MAMPs), pathogen-associated molecular patterns (PAMPs), and/or microbe-associated proteins.

The isolating step may be in accordance with a characteristic of the target microbe or microbe component or of the substrate or other bound capture molecule. Exemplary characteristics used for isolation may include size, mass, density, charge. In some aspects, the sample can be contacted with MTMs linked to a substrate. The engineered molecule can be a protein with engineered specificity for a particular microbe, microbe component, or class of either. The substrate to which the engineered molecules are linked or coupled can be an interior surface of a flow-through column, a bead, a magnetic particle, or any other known substrate used in target capture and separation. In certain aspects, the substrate may be a magnetic substrate or ELISA plate.

The step of isolating may include applying a magnet to the sample. For example, the engineered molecules described above may be linked to a magnetic particle such that application of a magnetic field to the sample can isolate the magnetic particles as well as the linked engineered molecule and any microbe or components bound thereto. Methods of the invention may use a superparamagnetic substrate. The magnetic substrate may comprise at least one of a magnetic bead, a superparamagnetic bead, or a magnetic microbead. In certain aspects, the MTM may be linked to an ELISA plate. Examples of magnetic capture techniques as well as ELISA-related substrates compatible with methods of the invention are described, for example, in U.S. Pat. Pub. 2015/0173883, already incorporated by reference in its entirety herein.

In certain aspects, isolating may include concentrating the microbe or microbe components of the sample. For example, after binding the target microbes or components thereof to a substrate using the engineered targeting molecules, the remaining sample, along with any unbound molecules can continue to flow out of the capture device or other substrate. The captured microbes or components thereof can then be washed in one or more steps to further remove unbound material. In various aspects, wash fluids may include calcium. The removal of unbound material allows for focused analysis of the target microbes and reduces the overall sample volume before any analysis steps.

To aid in binding of the microbe targeting molecule to the target microbe or microbe component and/or to prevent off-target binding and remove unbound material, the sample may be agitated and optionally heated. For example, the sample may be held at about 20° C. or more for about 1 minute or more, for example, up to about 20 minutes or up to about 30 minutes, to allow for microbe or microbe component binding.

Isolation can include elution of the microbe to release them from the bound substrate for further analysis. For example, after washing or otherwise removing unbound sample material, the remaining, captured target microbes can be eluted through a variety of known methods to allow for subsequent analysis steps without interference of the binding substrate or engineered targeting molecules. Elution may be accomplished through any known means and will generally depend on the desired analysis method and the composition of the substrate and engineered targeting molecule. Exemplary elution methods include temperature-based (e.g., heating to 70° C. or more), physical (e.g., agitation), photosensitive cleavage, or chemical methods. In certain aspects, elution through heating may be performed in calcium-free water. Exemplary chemical elution methods may involve a change in pH and/or application of a chelation agent. Chelation agents may include one or more of ethylenediaminetetraacetic acid (EDTA), calcium di sodium edetate (CaNa2EDTA), ethylene glycol-bis((3-aminoethyl ether)-N,N,N′,N′-tetraacetic acid (EGTA), 1,2-bis(o-aminophenoxy)ethane-N,N,N′,N′-tetraacetic acid (BAPTA), deferoxamine mesylate salt (DFOM).

After isolation of microbes or microbe components from the sample, the captured material can be digested for analysis. Digestion can refer to the release of constituent microbial components for subsequent analysis. Digestion can occur through exposure to a substance selected based on the desired analysis method and the target microbe to analyzed. In some aspects, that lysing or killing microbes in a sample by mechanical treatment (e.g., beadmilling, sonication, or other functionally equivalent method to disrupt cell wall), and/or chemical treatment (e.g., antibiotics, antivirals, antifungals or other antimicrobial agents) can allow detection of encapsulated microbes such as Klebsiella species that would not be otherwise detected. Thus, a pre-treatment of a sample to lyse or kill microbes can be performed prior to binding of the microbe-targeting molecules to exposed MAMPs. Therefore, this will not only increase the sensitivity of a microbe-targeting molecule-based detection method but can also surprisingly and significantly increase the spectrum of microbes that can be detected by an MTM-based detection method.

In some aspects, the patient has been treated with at least one antimicrobial agent. In some aspects, the sample contains at least one antibiotic or at least one antimicrobial agent, non-limiting examples of which are described further herein. In some aspects, the sample contains at least two antibiotics or at least two antimicrobial agents.

In some aspects, the patient has been treated with antibiotics, non-limiting examples of which are described further herein. In some aspects, the sample contains antibiotics, for example at least 1, at least 2, at least 3, at least 4, or at least 5 antibiotics.

In some aspects, the patient has been treated with antifungals, non-limiting examples of which are described further herein. In some aspects, the sample contains antifungals, for example at least 1, at least 2, at least 3, at least 4, or at least 5 antifungals.

In some aspects, the patient has been treated with antivirals, non-limiting examples of which are described further herein. In some aspects, the sample contains antivirals, for example at least 1, at least 2, at least 3, at least 4, or at least 5 antivirals.

Antibiotics can be from classes including Cephalosporin, Glycopeptide, Cyclic lipopeptide, Aminoglycoside, Macrolide, Oxazolidinone, Fluoroquinolones, Lincosamides, or Carbapenem. Antifungals can be from classes including Polyenes, Azoles, Nucleoside Analog, Echinocandin, or Allylamine. Antivirals can be from classes including CCR5 anatonists, Fusion inhibitors, Nucleoside/Nucleotide reverse transcriptase inhibitors (NRTIs), Non-nucleoside reverse transcriptase inhibitors (NNRTIs), Nucleotide reverse transcriptase inhibitors (NtRTIs), Integrase inhibitors, Protease inhibitors, DNA polymerase inhibitors, Guanosine analogs, Interferon-alpha, M2 ion channel blockers, Nucleoside inhibitors, NS5A polymerase inhibitors, NS3/4A protease inhibitors, Neuraminidase inhibitors, Nucleoside analogs, and Direct acting antivirals (DAAs).

In some aspects, the antimicrobial agent can be selected from aminoglycosides, ansamycins, beta-lactams, bis-biguanides, carbacephems, carbapenems, cationic polypeptides, cephalosporins, fluoroquinolones, glycopeptides, iron-sequestering glycoproteins, linosamides, lipopeptides, macrolides, monobactams, nitrofurans, oxazolidinones, penicillins, polypeptides, quaternary ammonium compounds, quinolones, silver compounds, sulfonamides, tetracyclines, and any combinations thereof. In some aspects, the antimicrobial agent can comprise an antibiotic.

Some exemplary specific antimicrobial agents include broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefmetazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin IV Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

Some exemplary antifungals include polyene antifungals, Amphotericin B, Candicidin, Filipin, Hamycin, Natamycin, Nystatin, Rimocidin, imidazole antifungals, triazole antifungals, thiazole antifungals, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Triazoles, Albaconazole, Efinaconazole, Epoxiconazole, Fluconazole, Isavuconazole, Itraconazole, Posaconazole, Propiconazole, Ravuconazole, Terconazole, Voriconazole, Abafungin, Allylamines, amorolfin, butenafine, naftifine, terbinafine, Echinocandins, Anidulafungin, Caspofungin, Micafungin, Aurones, Benzoic acid, Ciclopirox, Flucytosine, 5-fluorocytosin, Griseofulvin, Haloprogin, Tolnaftate, Undecylenic acid, Triacetin, Crystal violet, Castellani's paint, Orotomide, Miltefosine, Potassium iodide, Coal tar, Copper(II) sulfate, Selenium disulfide, Sodium thiosulfate, Piroctone olamine, Iodoquinol, clioquinol, Acrisorcin, Zinc pyrithione, and Sulfur. Additional antifungals known in the art can also be used.

Some exemplary antivirals agents include Abacavir, Acyclovir, Adefovir, Amantadine, Ampligen, Amprenavir, antiretroviral, Arbidol, Atazanavir, Atripla, Boceprevir, Cidofovir, Combivir, Daclatasvir, Darunavir, Delavirdine, Dasabuvir, Didanosine, Docosanol, Dolutegravir, Doravirine, Ecoliever, Edoxudine, Efavirenz, Elbasvir, Emtricitabine, Enfuvirtide, Entecavir, Etravirine, Famciclovir, Fomivirsen, Fosamprenavir, Foscarnet, Fosfonet, Fusion inhibitor, Ganciclovir, Gemcitabine, Glecaprevir, Grazoprevir, Ibacitabine, Idoxuridine, Imiquimod, Imunovir, Indinavir, Inosine, Integrase inhibitor, Interferon, Interferon type I, Interferon type II, Interferon type III, Lamivudine, Ledipasvir, Lopinavir, Lopiravir, Loviride, Maraviroc, Methisazone, Moroxydine, Nelfinavir, Nevirapine, Nexavir, Nitazoxanide, Norvir, Nucleoside analogues, Ombitasvir, Oseltamivir (Tamiflu), Paritaprevir, Peglyated Interferon-alpha, Peginterferon alfa-2a, Penciclovir, Peramivir, Pibrentasvir, Pleconaril, Podophyllotoxin, Protease inhibitor, Pyramidine, Raltegravir, Reverse transcriptase inhibitor, Ribavirin, Rilpivirine, Rimantadine, Ritonavir, Saquinavir, Simeprevir, Sofosbuvir, Stavudine, Synergistic enhancer (antiretroviral), Telaprevir, Telbivudine, Tenofovir, Tenofovir disoproxil, Tipranavir, Trifluridine, Trizivir, Tromantadine, Truvada, Valaciclovir (Valtrex), Valganciclovir, Velpatasvir, Vicriviroc, Vidarabine, Viramidine, Voxilaprevir, Zalcitabine, Zanamivir (Relenza), Zidovudine. Additional antivirals known in the art can also be used.

Without limitations, incubation of microbes present in the sample with one or more antimicrobial agents can be at any desired temperature and for any desired duration. In some aspects, the incubation can be performed at room temperature or at an elevated temperature. In some aspects, incubation can be performed at a temperature of about 30° C. to about 45° C. In one aspect, incubation can be performed at a temperature of about 37° C.

As indicated above, incubation of microbes present in a sample can be performed for any desired time period, which can vary with a number of factors, including but not limited to, temperature of incubation, concentration of microbes in the sample, and/or potency and/or concentrations of antimicrobial agents used. In some aspects, incubation can be for about at least one minute (e.g. one, five, ten, fifteen, twenty, twenty-five, thirty, thirty-five, forty, forty-five, fifty-five, sixty, ninety minutes or more). In some aspects, incubation can be for at least about one hour, at least about two hours, at least about three hours, at least about four hours, at least about five hours, at least about six hours, at least about seven hours, at least about eight hours, at least about nine hours, at least about ten hours or more. In some aspects, incubation can be for a period of about fifteen minutes to about ninety minutes. In one aspect, incubation can be for a period of about thirty minutes to about sixty minutes. In another aspect, incubation can be for a period of about thirty minutes to about twenty-four hours. In one aspect, incubation can be for a period of at least about four hours.

In some aspects, the pre-treatment can comprise incubating the sample with at least one or more degradative enzymes. For example, in some aspects, a degradative enzyme can be selected to cleave at least some of the cell wall carbohydrates, thus restoring detection of carbohydrates that are otherwise not recognized by MTMs. In some aspects, a degradative enzyme can be selected to cause call wall degradation and thus release or expose MAMPs that are otherwise unable bind to the MTMs. Other examples of degradative enzymes include, but are not limited to, proteases, lipases such as phospholipases, neuraminidase, and/or sialidase, or any other enzyme modifying the presentation of any MAMP to any MTM leveraged for detection of the MAMP. For instance, an exemplary MTM can comprise MBL or recombinant human MBL or engineered FcMBL, which binds mannose containing carbohydrates such as the core of LPS, the Wall Teichoic Acid from Staphylococcus aureus, PIM6 or Mannose-capped LipoArabinoMannan from M. tuberculosis whereas CRP binds phosphocholine found in Streptococcus pneumonia (Brundish and Baddiley, 1968), Haemophilus influenzae (Weiser et al., 1997), Pseudomonas aeruginosa, Neisseria meningitides, Neisseria gonorrhoeae (Serino and Virji, 2000), Morganella morganii (Potter, 1971), and Aspergillus fumigatus (Volanakis, “Human C-reactive protein: expression, structure, and function, “Molecular Immunology,” 2001, 38(2-3): 189-197). Other MTMs can be equally leveraged to recognize MAMPs such as nucleotide-binding oligomerization domains (NODs) or peptidoglycan recognition proteins (PGRP).

In some aspects, an antimicrobial mixture can be added during the digestion step where the antimicrobial mixture can include one or more antibiotics and/or one or more antifungals and/or one or more antivirals. Digesting the sample with a single antimicrobial, while enhancing the spectra, may cause variation in the spectra for a single pathogen between antimicrobial classes administered. Therefore, digesting the sample with an antimicrobial mixture, a normalized spectrum for each pathogen may be obtained, as shown in FIGS. 1-5.

In some aspects, the antimicrobial mixture may include one or more classes of antimicrobials including but not limited to: Cephalosporin, Glycopeptide, Cyclic lipopeptide, Aminoglycoside, Macrolide, Oxazolidinone, Fluoroquinolone, Lincosamide, Carbapenem; Echinocandin, or Polyene.

In some aspects, the antimicrobial mixture may include one or more of cefepime, vancomycin, daptomycin, amikacin, erythromycin, linezolid, ciproflaxin, lincomycin, meropenem, caspofungin or amphotericin. In a nonlimiting example, the antimicrobial mixture may include cefepime, vancomycin, daptomycin, amikacin, erythromycin, linezolid, ciproflaxin, lincomycin, meropenem, caspofungin and amphotericin. In another nonlimiting example, the antimicrobial mixture may include cefepime, vancomycin, daptomycin, amikacin, erythromycin, linezolid, ciproflaxin, lincomycin, and meropenem. In another nonlimiting example, the antimicrobial mixture may include caspofungin and amphotericin.

The antimicrobial mixture can include an antibiotic mixture at a concentration from about 0.1 ug/mL to about 100 mg/mL. The antimicrobial mixture may include an antifungal mixture at a concentration from about 0.01 ug/mL to about 100 mg/mL. The antimicrobial mixture may include an antiviral mixture at a concentration from about 0.01 ug/mL to about 100 mg/mL.

The amount of one or more antimicrobial agent added to a sample can be any desired amount and vary with a number of factors, including but not limited to, types of microbes in the sample, and/or potency of antimicrobial agents used. For example, one or more antimicrobial agents added to sample can have a concentration ranging from nanomolars to millimolars. In some aspects, one or more antimicrobial agents added to a sample can have a concentration ranging from 0.01 nM to about 100 mM, from about 0.01 nM to about 10 mM, or from about 0.1 nM to about 1 mM. In some aspects, one or more antimicrobial agents added to a sample can have a concentration ranging from nanograms per milliliters to micrograms per milliliters. In some aspects, one or more antimicrobial agents added to a sample can have a concentration ranging from about 1 ng/mL to about 1000 μg/mL, from about 10 ng/mL to about 750 μg/mL, or from about 100 ng/mL to about 500 μg/mL. In some aspects, one or more antimicrobial agents added to a sample can have a concentration ranging from about 10 μg/mL to about 500 μg/mL or from about 100 μg/mL to about 500 μg/mL.

Alternative to or in addition to the antimicrobial mixture, the substance used in digestion may include one or more enzymes, proteases, or carbohydrate-cleaving enzymes. In certain aspects, the substance used in the digestion can be trypsin. Digestion with a protease after isolation can standardize the isolated microbe or microbe components and can increase the probability of correctly identifying the microbe. In some aspects, the protease is selected from the group consisting of trypsin, chymotrypsin, pepsin, papain, elastase, or any combination thereof. The protease can also be any protease or protease mixture known in the art. Non-limiting examples of proteases include serine proteases, cysteine proteases, threonine proteases, aspartic proteases, glutamic proteases, metalloproteases, asparagine peptide lyases. In some aspects, the isolated microbe or microbe components are digested with at least one protease, at least 2 proteases, at least 3 proteases, at least 4 proteases, or at least 5 proteases, concurrently and/or sequentially. In some aspects, the protease is substantially free of protease inhibitors (e.g., 4-(2-Aminoethyl)benzenesulfonyl fluoride hydrochloride (AEBSF), Aprotinin, Bestatin, E64, Leupeptin, Pepstatin A).

In some aspects, the protease is trypsin. It is noted that trypsin is not commonly used in MALDI detection of microbes. In some aspects, the trypsin can be α-trypsin, β-trypsin, trypsin 1, trypsin 2, or mesotrypsin. In some aspects, the trypsin is at least 10% trypsin. As a non-limiting example, the trypsin is at least 1%, at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, or at least 90% trypsin. In some aspects, the trypsin is substantially free of trypsin inhibitors (e.g., Ca²⁺, Mg²⁺, heat, serpin, etc.). In some aspects, the trypsin comprises a divalent cation chelator (e.g., EDTA).

In some aspects, the isolated microbe or microbe component is digested for at most 30 seconds, at most 1 minute, at most 2 minutes, at most 3 minutes, at most 4 minutes, at most 5 minutes, at most 6 minutes, at most 7 minutes, at most 8 minutes, at most 9 minutes, at most 10 minutes, at most 20 minutes, at most 30 minutes, at most 40 minutes, at most 50 minutes, at most minutes, at most 70 minutes, at most 80 minutes, at most 90 minutes, at most 2 hours, at most 3 hours, at most 4 hours, at most 5 hours, at most 6 hours, at most 7 hours, at most 8 hours, at most 9 hours, at most 10 hours, at most 11 hours, or at most 12 hours. In some aspects, the isolated microbe or microbe component is digested overnight.

In some aspects, the isolated microbe or microbe component is digested at human body temperature (e.g., 36-38° C.). In some aspects, the isolated microbe or microbe component is digested at a temperature that is greater than 36-38° C. In some aspects, the digestion of the isolated microbe or microbe component further comprises heating the digestion. For example, heating the protease can permit faster digestion and can increase the probability of correctly identifying the microbe.

In some aspects, heating the digestion comprises microwave treatment. In some aspects, the microwave treatment of the digestion is at a power of least 500 watts (W), at least 600 W, at least 700 W, at least 800 W, at least 900 W, at least 1000 W, at least 1100 W, at least 1200 W, at least 1300 W, at least 1400 W, or at least 1500 W. In some aspects, the microwave treatment of the digestion occurs for 1 minute. As a non-limiting example, the microwave treatment of the digestion can occur for at most 10 seconds, at most 20 seconds, at most 30 seconds, at most 40 seconds, at most 50 seconds, at most 1 minute, at most 2 minutes, at most 3 minutes, at most 4 minutes, at most 5 minutes, at most 6 minutes, at most 7 minutes, at most 8 minutes, at most 9 minutes, or at most 10 minutes.

In some aspects, the method described herein further comprises contacting the digested microbe or microbe components with a composition that is more acidic than the digested microbe or microbe components (e.g., said step of contacting can decrease the pH of the solution). As used herein, “more acidic” refers to a composition or solution with a lower pH compared to another composition or solution. Contacting the digested microbe or microbe components with such a composition can quickly and effectively quench the protease digestion reaction, increase component stability, and improve mass spectrometry (e.g., MALDI) sensitivity.

In some aspects, the composition that is more acidic than the digested microbe or microbe components is present at a volume equal to or greater than the volume of the digested microbe or microbe components. As a non-limiting example, the volume of the composition that is more acidic than the digested microbe or microbe components can be present at a 1:1, 5:4, 4:3, 3:2, 2:1 ratio to the volume of the digested microbe or microbe components.

In some aspects, the composition that is more acidic than the digested microbe or microbe components is present at a concentration of at least 0.5%. As a non-limiting example, the concentration of the composition that is more acidic than the digested microbe or microbe components can be at least 0.1%, at least 0.2%, at least 0.3%, at least 0.4%, at least 0.5%, at least at least 0.7%, at least 0.8%, at least 0.9%, at least 1.0%, at least 2.0%, at least 3.0%, at least 4.0%, at least 5.0%, at least 6.0%, at least 7.0%, at least 8.0%, at least 9.0%, or at least 10.0%

In some aspects, the composition that is more acidic than the digested microbe or microbe components is selected from the group consisting of trifluoroacetic acid (TFA; CF₃COOH), acetic acid (CH₃COOH), and formic acid (CH₃COOH). As a non-limiting example, the composition that is more acidic than the digested microbe or microbe components can be hydrofluoric acid (HF), phosphoric acid (H₃PO₄), nitrous acid (HNO₂), lactic acid, citric acid, oxalic acid, uric acid, malic acid, or any carboxylic acid (—COOH). As a non-limiting example, the composition that is more acidic than the digested microbe or microbe components can be hydrochloric acid (HCl), nitric acid (HNO₃), sulfuric acid (H₂SO₄), hydrobromic acid (HBr), hydroiodic acid (HI), perchloric acid (HClO₄), or chloric acid (HClO₃). As a non-limiting example, the composition that is more acidic than the digested microbe or microbe components can be any composition with a pH below 7.

In some aspects, the isolated microbe or microbe components are digested with a protease but not heated and not contacted with a composition that is more acidic than the digested microbe or microbe components. In some aspects, the isolated microbe or microbe components are digested with a protease and heated but not contacted with a composition that is more acidic than the digested microbe or microbe components. In some aspects, the isolated microbe or microbe components are digested with a protease and contacted with a composition that is more acidic than the digested microbe or microbe components but not heated. In some aspects, the isolated microbe or microbe components are digested with a protease, heated, and contacted with a composition that is more acidic than the digested microbe or microbe components. In some aspects, the isolated microbe or microbe components are not digested with a protease, not heated, and not contacted with a composition that is more acidic than the digested microbe or microbe components.

In some aspects, the sample has not been cultured. In other words, the microbes in the sample have not been allowed to replicate or amplify in a culture medium. Accordingly, in some aspects, the methods described herein do not comprise a culturing step, e.g., a step involving culturing and/or maintaining the microbe(s) ex vivo or in vitro. In some aspects, the time from the step of collecting the sample to the end of detection takes equal to or less than 90 minutes. As a non-limiting example, the time from the step of collecting the sample to the end of detection takes at most 60 minutes, at most 70 minutes, at most 80 minutes, at most 90 minutes, at most 100 minutes, at most 110 minutes, at most 120 minutes, at most 2.5 hours, at most 3.0 hours, at most 3.5 hours, at most 4.0 hours, at most 4.5 hours, at most 5.0 hours, at most 5.5 hours, at most 6.0 hours, at most 12.0 hours, at most 18 hours, or at most 24 hours.

In various aspects, microbes may be contacted with a matrix or matrix solution. The substrate can be evenly sprayed with matrix solution prior to analyzing the microbe or microbe components to generate a homogenous layer of crystallized matrix on top of the substrate. The application of a crystallized matrix can assist in certain analysis techniques including MALDI mass spectrometry. The desired matrix consists of crystallized molecules such as 3,5-dimethoxy-4-hydroxycinnamic acid (sinapinic acid), α-cyano-4-hydroxycinnamic acid (α-CHCA, alpha-cyano or alpha-matrix) and 2,5-dihydroxybenzoic acid (DHB). A solution of one of these molecules is made, often in a mixture of highly purified water and an organic solvent such as acetonitrile (ACN) or ethanol. Trifluoroacetic acid (TFA), as discussed above, can be used as a counter ion source. An exemplary matrix-solution is 20 mg/mL sinapinic acid in CAN at a ratio of 50:50:0.1 with water and TFA.

The application of a laser energy absorbing matrix in the sample preparation allows for the application of ionization-based analysis techniques (e.g., mass spectrometry) with minimal fragmentation. By applying a matrix directly to captured target microbes separated from sample, accurate microbe characterization can be carried out with minimal steps and delay. Accordingly, actionable results can be quickly obtained leading to quicker treatments and better patient outcomes. The use of a crystallized matrix is particularly useful in the analysis of biomolecules such as microbes and components thereof, which tend to be fragile and fragment when ionized by conventional ionization methods.

Detection and/or Identification of Microbes

While microbes and microbial components may be detected and/or identified directly through the use of the diagnostic, therapeutic or filtration devices of the invention, in some cases additional means may be needed to detect and/or identify captured microbes and microbial components, whether obtained using detection, therapeutic or filtration devices.

Suitable identification means for detecting and/or identifying microbes and microbial components include, but are not limited to, volatile organic compound methods, spectrometry (e.g., Raman spectroscopy; FFT (Fast-Fourier Transform); Fourier-Transform Infrared Spectroscopy (FTIR); infrared spectrometry; Nuclear Magnetic Resonance (NMR) spectrometry), electrochemical detection, polynucleotide detection, fluorescence anisotropy, fluorescence resonance energy transfer, electron transfer, enzyme assay, magnetism, electrical conductivity, electrochemical detection, isoelectric focusing, lateral flow assay (LFA), microfluidics, amino acid sequence, nucleic acid sequencing, flow cytometry, chromatography, immunoprecipitation, immunoseparation, aptamer binding, filtration, electrophoresis, use of a CCD camera, immunoassay, ELISA, Gram staining, immunostaining, microscopy, immunofluorescence, size/weight/charge detection, CRISPR, western blot, polymerase chain reaction (PCR), RT-PCR, isothermal amplification, sequencing, next gen sequencing, fluorescence in situ hybridization, sequencing, mass spectrometry, SPR, LSPR, or substantially any combination thereof. The captured microbe can remain bound on the MTM-coated solid substrates during detection and/or analysis, or be isolated form the MTM-coated solid substrates prior to detection and/or analysis.

In some aspects, the microbes and/or microbial components (e.g., MAMPs) bound to MTM-coated (e.g., lectin-coated) solid substrates (e.g., polymeric or magnetic particles or beads) can be detected by ELLecSA. Additional information various aspects of FcMBL based assays can be found, e.g., in PCT application publications WO 2013/012924 and WO 2013/130875, the contents of all of which are incorporated herein by reference in their entireties.

In some aspects, the identification means for detection/identification can be performed very quickly using magnetic beads. Pathogens can be captured directed from the blood by MTM-coated magnetic beads and mass spectroscopy (MS) can be conducted. Such a method eliminates an elution step and put beads directly onto a plate for MS analysis. This method allows: microbe detection within 5 minutes of sample collection (improves by 20 min); higher concentration/improved sensitivity; smaller beads and more surface area so more sample.

In another non-limiting example, a method for detecting microbes and microbe components comprises preparing the sample. In some aspects, when qPCR is used, preparing a sample can comprise filtering the sample, extracting the mRNA, microRNA, DNA, preparing a standard curve, and preparing a master mix. For example, the sample can be mixed with a sterile phosphate buffer saline solution, filtered, for example using a vacuum, and placed into one or more tubes comprising glass beads. Next, a buffer can be prepared and added to the tubes. In some aspects, the buffer comprises an AE buffer comprising 10 mM Tris-Cl and 0.5 mM EDTA at pH 9.0. Next, the tubes can be placed in a centrifuge. Optionally, the liquid from each tube can be placed into a fresh tube and placed in the centrifuge again. The centrifuge can be any suitable centrifuge as is known in the art. Next, a standard curve can be prepared. A qPCR master mix can be prepared. For example, deoxyribonucleotide triphosphate (dNTP), qPCR Buffer, DNA polymerase, and primers in bead form can be mixed. In some aspects, one or more dNTPs can comprise a different phosphate group, for example, purines and pyrimidines. Any suitable primer can be used as is known in the art. Next, nuclease free water can be added to the master mix. The master mix can be placed in a vortex to dissolve the beads and then placed in a centrifuge to evenly disperse the mixture.

In some aspects, when RT-PCR is used, preparing a sample can comprise preparing a solution including the sample, incubating the solution, preparing a reverse transcription mix, and placing the mixture in a thermocycler machine. The thermocycler machine can be any suitable thermocycler machine as is known in the art. For example, a solution can be prepared including the sample, primer, sample RNA, dNTPs and diethyl pyrocarbonate (DEPC)-treated water. In some aspects, one or more dNTPs can comprise a different phosphate group, for example, purines and pyrimidines. Next, the solution can be incubated to denature the RNA. Next, the reverse transcription mix can be prepared, for example, cDNA synthesis buffer, dithiothreitol (DTT), a recombinant ribonuclease inhibitor, DEPC-treated water, a reverse transcription enzyme, denatured RNA, and primer can be mixed. Then, the mixture is placed in a thermocycler machine. The thermocycler machine can be any suitable thermocycler machine as is known in the art.

Methods for detecting the microbe or microbe components can be via polymerase chain reaction (PCR). Any type of PCR can be used, including but not limited to: Amplified fragment length polymorphism (AFLP) PCR; Allele-specific PCR; Alu PCR; Assembly PCR; Asymmetric PCR; COLD PCR; Colony PCR; Conventional PCR; Digital PCR (dPCR); Fast-cycling PCR; High-fidelity PCR; High-Resolution Melt (HRM) PCR; Hot-start PCR; In situ PCR; Intersequence-specific (ISSR) PCR; Inverse PCR; LATE (linear after the exponential) PCR; Ligation-mediated PCR; Long-range PCR; Methylation-specific PCR (MSP); Miniprimer PCR; Multiplex-PCR; Nanoparticle-Assisted PCR (nanoPCR); Nested PCR; Overlap extension PCR; Real-Time PCR (quantitative PCR or qPCR); Repetitive sequence-based PCR; Reverse-Transcriptase (RT-PCR); Reverse-Transcriptase Real-Time PCR (RT-qPCR); RNase H-dependent PCR (rhPCR); Single cell PCR; Single Specific Primer-PCR (SSP-PCR); Solid phase PCR; Suicide PCR; Thermal asymmetric interlaced PCR (TAIL-PCR); Touch down (TD) PCR; Variable Number of Tandem Repeats (VNTR) PCR.

In some aspects, the detection method comprises real-time PCR or qPCR. In some aspects, the detection method comprises RT-PCR. In some aspects, the method uses radioactive isotope markers to detect the microbe or microbe components. In some aspects, the method uses fluorescent dyes to detect the microbe or microbe components. In some aspects, the signals from the PCR method can be compared to a library and the microbe can be identified. The PCR machine can be any suitable PCR machine as is known in the art, and the sample can be processed by the PCR machine via any suitable method as is known in the art.

In some aspects, the detection method comprises plasmon resonance (PR), such as surface plasmon resonance (SPR) and localized surface plasmon resonance (LSPR), which is highly sensitive to changes that occur at the interface between a metal and a dielectric medium (e.g. water, air, etc). PR technology utilizes surface plasmons (electromagnetic waves) that can be excited at certain metal interfaces, for example, graphene silver and gold. When incident light is coupled with the metal interface at angles greater than the critical angle, the reflected light exhibits a sharp attenuation. An SPR device comprises an optical biosensor that measures binding events of biomolecules at a metal surface by detecting changes in the local refractive index. Thus, when a particle binds to the surface of the sensor and interacts with the surface motion, the resonance frequency of the transduced signal is altered. For the binding of small nanoscale particles, such as nucleic acids, polypeptides or proteins, the addition of mass approximately corresponds to an increase of the thickness of the surface/substrate and the resonance frequency decreases.

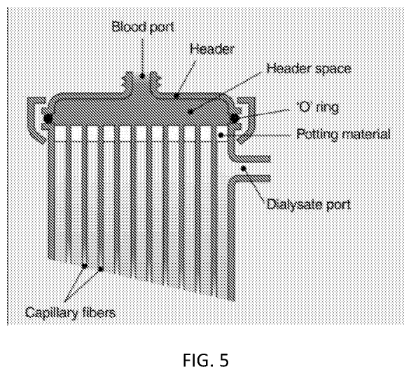

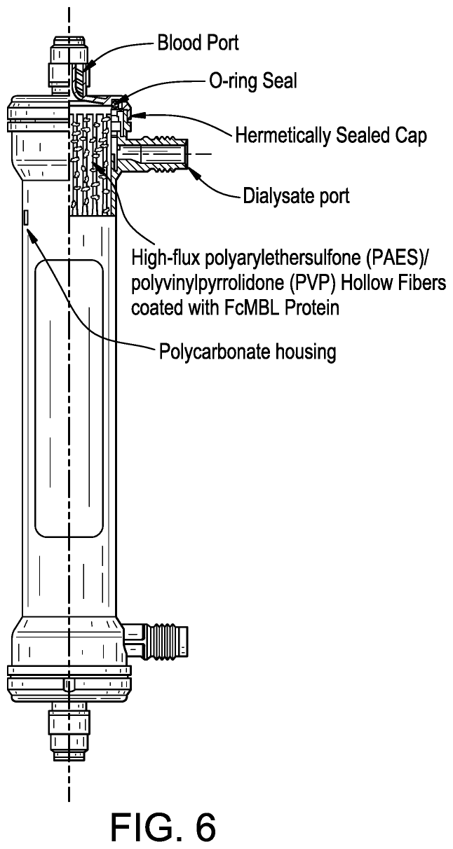

Because SPR and LSPR sensors are based on simple optical components, including a light source, a detector, optics, microfluidics and surface chemistry, they can be integrated into portable diagnostic devices. Exemplary systems are shown in FIGS. 5-7. In the case of SPR, the plasmonic material can comprise a gold film, which requires a prism or waveguide to excite the plasmon, while colloidal nanostructures or nanostructured films are directly excited in LSPR. MTMs can be attached to these nanostructures. In addition to the use of gold and silver as surfaces for SPR, graphene (GN) may be used. GN is a single layer, two-dimensional structure nanomaterial that exhibits exceptional physical, electrical and chemical properties. The unique parameters of GN are electron mobility, thermal conductivity, high surface area and electrical conductivity.

In some aspects, analysis is performed via a mass spectrometric method. Mass spectrometric methods can include at least one of electron ionization, chemical ionization, electrospray ionization, atmospheric pressure chemical ionization, and matrix-assisted laser desorption ionization (MALDI). Molecules may be ionized for mass spectrometry by any method known in the art, such as ambient ionization, chemical ionization (CI), desorption electrospray ionization (DESI), electron impact (EI), electrospray ionization (ESI), fast-atom bombardment (FAB), field ionization, laser ionization (LIMS), matrix-assisted laser desorption ionization (MALDI), paper spray ionization, plasma and glow discharge, plasma-desorption ionization (PD), resonance ionization (RIMS), secondary ionization (SIMS), spark source, or thermal ionization (TIMS). Methods of mass spectrometry are known in the art and described in, for example, U.S. Pat. Nos. 8,895,918; 9,546,979; 9,761,426; Hoffman and Stroobant, Mass Spectrometry: Principles and Applications (2nd ed.). John Wiley and Sons (2001), ISBN 0-471-48566-7; Dass, Principles and practice of biological mass spectrometry, New York: John Wiley (2001) ISBN 0-471-33053-1; and Lee, ed., Mass Spectrometry Handbook, John Wiley and Sons, (2012) ISBN: 978-0-470-53673-5, the contents of each of which are incorporated herein by reference. Exemplary mass spectrometers are manufactured by Bruker. The mass spectrometric method can be automated.

Mass spectrometry or other analysis of captured microbes or microbial components can be used to identify the species and/or strain of microbe. Such identification is particularly useful where the microbe is a pathogen. Human pathogenic and other microbes or components thereof can be identified using mass spectrometry results as described in Singhai, et al., 2015, MALDI-TOF mass spectrometry: an emerging technology for microbial identification and diagnosis, Front Microbiol., 6:791, incorporated herein by reference.

Analysis via MALDI mass spectrometry comprises first contacting a substrate with a matrix or matrix solution. The substrate can be evenly sprayed with matrix solution prior to analyzing the microbe or microbe components to generate a homogenous layer of crystallized matrix on top of the substrate. The application of a crystallized matrix can assist in certain analysis techniques including MALDI mass spectrometry. The desired matrix consists of crystallized molecules such as 3,5-dimethoxy-4-hydroxycinnamic acid (sinapinic acid), α-cyano-4-hydroxycinnamic acid (α-CHCA, alpha-cyano or alpha-matrix) and 2,5-dihydroxybenzoic acid (DHB). A solution of one of these molecules is made, often in a mixture of highly purified water and an organic solvent such as acetonitrile (ACN) or ethanol. Trifluoroacetic acid (TFA), as discussed above, can be used as a counter ion source. An exemplary matrix-solution is 20 mg/mL sinapinic acid in CAN at a ratio of 50:50:0.1 with water and TFA.

The application of a laser energy absorbing matrix in the fluid preparation allows for the application of ionization-based analysis techniques (e.g., mass spectrometry) with minimal fragmentation. By applying a matrix directly to captured target microbes separated from fluid, accurate microbe characterization can be carried out with minimal steps and delay. Accordingly, actionable results can be quickly obtained leading to quicker treatments and better patient outcomes. The use of a crystallized matrix is particularly useful in the analysis of biomolecules such as microbes and components thereof, which tend to be fragile and fragment when ionized by conventional ionization methods.

A smartphone can serve as a point-of-care (POC) device or used in the field, for example, to detect airborne or water pathogens. Such a device would have both civilian and military applications. For example, the invention includes methods for screening a traveler for infectious disease status by having the traveler exhale into a diagnostic device of the invention, which is diagnostic for at least one infectious agent. The device can determine the infectious disease status of a traveler prior to the traveler undertaking a journey. Allowing the results of the diagnostic test to be reported prior to or contemporaneous with the arrival of the traveler at the traveler's destination.

Integrating MTM technology with local/surface plasmon resonance (L/SPR) enables POC and in-field monitoring. Advantages of the invention comprising L/SPR-based diagnostic devices include: fast and label-free direct detection. As an example, use of such a device comprises the immobilization of pathogen-specific antibodies to the surface of the link layer at fixed concentrations, allowing the MTM-captured pathogen to bind to the fixed antibody or aptamer. Free flowing MTM-microbe complexes are allowed to bind to the pathogen-specific antibody. Known concentrations of representative MTMs and the relative resonance units (RU) of SPR are used to establish standard curves. For comparison purposes, the SPR sample may be compared with results generated by an ELISA. Risk estimates can be provided on all infectious disease screens. For example, the risk for sepsis is based upon the level of PAMPS/MAMPS, PCT, PSP, IL-6, and CRP. Military applications include rapid screening of pathogens in the field.

### Antibiotic Susceptibility Testing

In addition to identification of microbes or microbe components, mass spectrometry or other analysis of captured microbes or microbial components can be used to determine the antibiotic susceptibility of the captured microbe allowing infected patients to receive the most effective treatment with minimal delay, thereby reducing the risk of complications such as septic shock. For example, a method of determining an antimicrobial susceptibility of a microbe can include collecting at least one biological sample, such as bodily fluid, from a source comprising at least one microbe or microbe component, preparing the at least one sample, and performing an antimicrobial susceptibility test.

Preparing the at least one sample, as described herein, can include contacting the sample with an MTM, for example, FcMBL, linked to a substrate, isolating the microbe or microbe components bound to the MTM, digesting the isolated microbe or microbe components with a substance, and contacting the microbe or microbe components with a matrix or matrix solution on a target substrate.

An antimicrobial susceptibility test can include obtaining a first signal from the sample comprising at least one microbe or microbe component, obtaining a second signal from the sample comprising the at least one microbe or microbe component and at least one antimicrobial; and comparing the first and second signal, where if the difference between the first and second signals is greater than a determined threshold, the at least one microbe is susceptible to the at least one antimicrobial, and wherein the first and second signals are obtained using a mass spectrometry method. The at least one antimicrobial is provided to the sample after the first signal is obtained.

The source can be a human or an animal. The sample can be a bodily fluid of a human or an animal. The fluid can include a buffer solution.

The method can include collecting a second sample from a human after the human is treated with at least one antimicrobial, where the second signal is obtained from the second sample, and where if the difference between the first and second signals is greater than a determined threshold, the at least one microbe is susceptible to the at least one antimicrobial. The second sample can be collected 24 hours or less after the first sample.

In some aspects, the second signal can be compared to a signal library, where the library comprises a signal profile for each of a plurality of microbes, and if the difference between the second signal and any of the plurality of microbe signal profiles is less than a determined threshold, the at least one microbe is susceptible to at least one antimicrobial identified in at least one of the plurality of microbe signal profiles. The first and second signals can be entered into a signal library.

The method can include inoculating the sample for 24 hours or less after the first signal is obtained and obtaining a third signal from the inoculated sample, where if the difference between the first and third signals is greater than a determined threshold, the sample comprises at least one live microbe. Additionally, if the difference between the first and second signals is greater than a determined threshold, the at least one microbe is susceptible to the at least one antimicrobial.

The method can include inoculating the sample for greater than 24 hours after the first signal is obtained and obtaining a third signal from the inoculated sample, where if the difference between the first and third signals is greater than a determined threshold, the sample comprises at least one live microbe. Additionally, if the difference between the first and second signals is greater than a determined threshold, the at least one microbe is susceptible to the at least one antimicrobial.

In addition to or alternative to performing an antimicrobial susceptibility test, the presence of an antimicrobial resistance marker and/or the absence of an antimicrobial susceptibility marker can be determined to indicate that the at least one microbe in a sample is resistant to that specific antimicrobial. In some aspects, the absence of an antimicrobial resistance marker and/or the presence of an antimicrobial susceptibility marker can indicate that the at least one microbe in a sample is susceptible to that specific antimicrobial. The detection methods described herein can be used to determine the presence or absence of an antimicrobial resistance marker or an antimicrobial susceptibility marker.

As used herein “antibiotic resistance marker” refers to a gene product, mRNA, polypeptide, polypeptide variant, or other macromolecule that confers resistance to a specific antimicrobial, such as by enzymatically cleaving the antimicrobial or specifically effluxing the antimicrobial. In some aspects, non-limiting examples of antimicrobial resistance markers include Aminocoumarin-resistant DNA topoisomerases (e.g., Aminocoumarin-resistant GyrB, ParE, ParY); Aminoglycoside acetyltransferases (e.g., AAC(1), AAC(2′), AAC(3), AAC(6′)); Aminoglycoside nucleotidyltransferases (e.g., ANT(2″), ANT(3″), ANT(4′), ANT(6), ANT(9)); Aminoglycoside phosphotransferases (e.g., APH(2″), APH(3″), APH(3′), APH(4), APH(6), APH(7″), APH(9)); 16S rRNA methyltransferases (e.g., ArmA, RmtA, RmtB, RmtC, Sgm); Class A β-lactamases (e.g., AER, BLA1, CTX-M, KPC, SHV, TEM, etc.); Class B (metallo-)β-lactamases (e.g., BlaB, CcrA, IMP, NDM, VIM, etc.); Class C β-lactamases (e.g., ACT, AmpC, CMY, LAT, PDC, etc.); Class D β-lactamases (e.g., OXA (3-lactamase); mecA (methicillin-resistant PBP2); mutant porin proteins conferring antibiotic resistance; antibiotic-resistant Omp36, antibiotic-resistant OmpF, antibiotic-resistant PIB (por); genes modulating β-lactam resistance (e.g., bla (blaI, blaR1) and mec (mecI, mecR1) operons); Chloramphenicol acetyltransferase (CAT); Chloramphenicol phosphotransferase; Ethambutol-resistant arabinosyltransferase (EmbB); Mupirocin-resistant isoleucyl-tRNA synthetases (e.g., MupA, MupB); resistance markers for peptide antibiotics, including but not limited to integral membrane protein MprF; resistnace markers for phenicol, including but not limited to Cfr 23S rRNA methyltransferase; Rifampin ADP-ribosyltransferase (Arr); Rifampin glycosyltransferase; Rifampin monooxygenase; Rifampin phosphotransferase; Rifampin resistance RNA polymerase-binding proteins (e.g., DnaA, RbpA); Rifampin-resistant beta-subunit of RNA polymerase (RpoB); resistance markers against Streptogramins; Cfr 23S rRNA methyltransferase; Erm 23S rRNA methyltransferases (e.g., ErmA, ErmB, Erm(31), etc.); Streptogramin resistance ATP-binding cassette (ABC) efflux pumps (e.g., Lsa, MsrA, Vga, VgaB); Streptogramin Vgb lyase; Vat acetyltransferase; Fluoroquinolone acetyltransferase; Fluoroquinolone-resistant DNA topoisomerases; Fluoroquinolone-resistant GyrA, Fluoroquinolone-resistant GyrB, Fluoroquinolone-resistant ParC; Quinolone resistance protein (Qnr); Fosfomycin phosphotransferases (e.g., FomA, FomB, FosC); Fosfomycin thiol transferases (e.g., FosA, FosB, FosX); resistance markers against Glycopeptides, including not limited to VanA, VanB, VanD, VanR, VanS, etc.; resitance markers against Lincosamides; Cfr 23S rRNA methyltransferase; Erm 23S rRNA methyltransferases (e.g., ErmA, ErmB, Erm(31), etc.); Lincosamide nucleotidyltransferase (Lin); resistance markers against Linezolid; Cfr 23S rRNA methyltransferase; resisntance markers against Macrolides, such as Cfr 23S rRNA methyltransferase, Erm 23S rRNA methyltransferases (e.g., ErmA, ErmB, Erm(31), etc.); Macrolide esterases (e.g., EreA, EreB); Macrolide glycosyltransferases (e.g., GimA, Mgt, Ole); Macrolide phosphotransferases (MPH) (e.g., MPH(2′)-I, MPH(2′)-II); Macrolide resistance efflux pumps (e.g., MefA, MefE, Mel); Streptothricin acetyltransferase (sat); Sulfonamide-resistant dihydropteroate synthases (e.g., Sul1, Sul2, Sul3, sulfonamide-resistant FolP); resistance markers against Tetracyclines; mutant porin PIB (por) with reduced permeability; Tetracycline inactivation enzyme TetX; Tetracycline resistance major facilitator superfamily (MFS) efflux pumps (e.g., TetA, TetB, TetC, Tet30, Tet31, etc.); Tetracycline resistance ribosomal protection proteins (e.g., TetM, TetO, TetQ, Tet32, Tet36, etc.); efflux pumps conferring antibiotic resistance: ABC antibiotic efflux pump (e.g., MacAB-TolC, MsbA, MsrA, VgaB, etc.); MFS antibiotic efflux pump (e.g., EmrD, EmrAB-TolC, NorB, GepA, etc.); multidrug and toxic compound extrusion (MATE) transporter (e.g., MepA); resistance-nodulation-cell division (RND) efflux pump (e.g., AdeABC, AcrD, MexAB-OprM, mtrCDE, etc.); small multidrug resistance (SMR) antibiotic efflux pump (e.g., EmrE); genes modulating antibiotic efflux (e.g., adeR, acrR, baeSR, mexR, phoPQ, mtrR, etc.). See e.g., MacAuthur et al., Antimicrob Agents Chemother. 2013 July; 57(7):3348-57, which is incorporated herein by reference. In some aspects, an antimicrobial resistance marker can include any protein, polypeptide, polypeptide variant, or other macromolecule known in the art to confer resistance to a specific antimicrobial or family of antimicrobials.

As used herein “antibiotic susceptibility marker” refers to a gene product, mRNA, polypeptide, polypeptide variant, or other macromolecule that confers susceptibility to a specific antimicrobial, especially in a domain at fashion. In some aspects, an antibiotic susceptibility marker can include any mutant or variant of one of the aforementioned antibiotic resistance markers comprising a mutation that reduces or eliminates the antibiotic resistance. In some aspects, non-limiting examples of antimicrobial susceptibility markers include RpsL and GyrA conferring sensitivity in a dominant fashion to two antibiotics, streptomycin and nalidixic acid, respectively (see e.g., Edgar et al., Appl Environ Microbiol. 2012 February; 78(3): 744-751). In some aspects, an antimicrobial susceptibility marker can include any protein, polypeptide, polypeptide variant, or other macromolecule known in the art to confer susceptibly to a specific antimicrobial or family of antimicrobials.

### Therapeutic Devices

As suggested above, MTMs and compositions of the invention can be used in therapeutic devices (and related methods) to treat microbial infections and diseases and related conditions in a subject. In any of these aspects, the device can be configured for use in a clinical setting or an in-home setting.

The therapeutic devices of the present invention are limited only in that (i) they are devices (or components thereof) having therapeutic attributes or that can be used in therapeutic systems, and (ii) they contain one or more MTMs of the invention. In a typical example, the therapeutic devices or at least a component thereof will be coated with MTMs or otherwise display MTMs on a surface of the device or component thereof.

The therapeutic devices of the invention include, but are not limited to, the following: oxygenation devices, extracorporeal devices (e.g. ECMO devices), blood pump devices, heart-lung devices, dialysis devices, drainage devices, blood transfusion devices, infusion devices, temperature management devices, pressure management devices, plasma separators, hemoperfusion cartridges, adsorbent devices, monitoring devices, cytokine reduction systems, pathogen reduction systems, PAMP reduction systems, respiration devices, ventilation devices, and catheters or tubes used in a medical procedure. Relevant devices include both those located externally and those located internally to the body of the subject (e.g. central venous lines). At least one surface of the device is coated with MTMs of the invention, or otherwise displays MTMs such that the MTMs are exposed to biological sample under conditions permitting binding of microbes or microbial components in the sample by the MTMs.

Alternatively, or in addition, the therapeutic devices of the invention comprise at least one component that is coated with MTMs of the invention, or otherwise display MTMs such that the MTMs are exposed to biological sample under conditions permitting binding of microbes or microbial components in the sample by the MTMs. Such components include, but are not limited to, supports (e.g. graphene), beads (e.g. gold particles), particles (including nanoparticles, microparticles, polymer microbeads, magnetic microbeads, and the like), filters, fibers, screens, mesh, tubes, hollow fibers, scaffolds, plates, channels, magnetic materials, medical apparatuses (e.g., needles or catheters) or implants, dipsticks or test strips, filtration devices or membranes, cartridges (e.g. hollow fiber cartridges), microfluidic devices, mixing elements (e.g., spiral mixers), extracorporeal devices, and other substrates commonly utilized in therapeutic applications, and any combinations thereof. In some aspects, the therapeutic device or component thereof is a solid substrate, such as a filter or cartridge.

Examples of materials that can be used in the components of the therapeutic devices include fluoropolymer immobilized liquid perfluorocarbon (FILP); polyurethane PICC surfaces, such as poly(bis(trifluoroethoxy), phosphazene-coated COBRA PzF18 and poly(vinylidene fluoride-co-hexafluoropropylene)-coated XIENCE Sierra coronary stents; AngioDynamics BioFlo (endexo) PICC catheters and CerebroFlo (endexo) extraventricular drain catheters.

When the therapeutic device or component thereof is a filter, the filter may be further coated with a material such as Endexo® surface modifying macromolecules, which may decrease blood clot formation and provide additional PAMP- and bacteria-depleting functionality need for dialysis patients with PAMPEMIA and/or blood infections.

The therapeutic devices of the invention may be used in a wide variety of therapeutic applications including, but not limited to, methods of treating microbial infections in a subject. Such methods include contacting a bodily fluid of the subject with a therapeutic device of the invention under conditions that permit binding of microbes by MTMs displayed by the therapeutic device, thus reducing the amount of microbes in the bodily fluid of the subject. In one aspect, the microbial infection is a bacterial infection. In another aspect, the microbial infection is a viral infection. In further aspect, the microbial infection is a fungal infection. Such methods can be used to treat infectious diseases.

The therapeutic devices of the invention may be used in therapeutic applications that remove microbial components from a subject. Such subjects may not have an active microbial infection, but may be suffering from the effects of the continued presence of microbial components. For example, clearing residual PAMPs (e.g. DAMPs) from the blood could decrease the amount of organ damage caused by microbial components through inflammation. Such methods include contacting a bodily fluid of the subject with a therapeutic device of the invention under conditions that permit binding of microbial components by MTMs displayed by the therapeutic device, thus reducing the amount of microbial components in the bodily fluid of the subject. Such methods can also be used to “scrub” non-self substances from the blood, with the “clean” blood being returned to the subject or donating for use in a different subject or assayed. In such applications, the therapeutic devices could be the filter of a hemodialysis device and/or even simply the tubing or flow path that conveys the blood through the device.

It should be understood that the therapeutic devices of the invention may also be used in therapeutic applications that are not directed to the capture of microbes of microbial components. For example, sterile inflammation is a type of pathogen-free inflammation caused by mechanical trauma, ischemia, stress or environmental conditions such as ultra-violet radiation. These damaging factors induce the secretion of molecular agents collectively termed danger-associated molecular patterns (DAMPs). DAMPs are recognized by immune receptors, such as toll-like receptors (TLRs) and NOD-like receptor family, pyrin domain containing 3 (NLRP3), expressed by sentinel cells of the immune system. The therapeutic devices of the invention can be used to reduce and/or remove DAMPs from the blood of a subject. Such devices include a filter having at least one surface coated with or otherwise displaying MTMs of the invention. Methods using these devices include contacting a bodily fluid of the subject with a therapeutic device of the invention under conditions that permit binding of DAMPs by MTMs displayed by the therapeutic device, thus reducing the amount of DAMPs in the bodily fluid of the subject.

Another example of a non-microbial condition that may be treated using the therapeutic devices of the invention includes chronic inflammation disorder, which is characterized by the accumulation of modified lipoproteins in the arterial intima. This disorder can be treated using the therapeutic devices of the invention. MTM-coated therapeutic devices can be used to clear atherogenic lipoproteins (which are bound by the MTMs) from the blood, which decreases atherosclerotic disease. Subjects undergoing hemodialysis have higher rates of cardiovascular morbidity and mortality compared to the general population. Therefore, such a therapy would be particular helpful in subjects having acute kidney injury (AKI) or end-stage renal disease (ESRD). Various reports suggest that functional MBL levels change following dialysis and this change could increase atherosclerotic lesions. Studies in Cl^{-/-} and MBL^{-/-} mice suggest that these molecules play a protective role in the early atherosclerotic lesion and conventional dialysis system depletes MBL and C1q. Such devices include a filter having at least one surface coated with or otherwise displaying MTMs of the invention. Methods of using such devices include contacting a bodily fluid of the subject with a therapeutic device of the invention under conditions that permit binding of atherogenic lipoproteins by MTMs displayed by the therapeutic device, thus reducing the amount of atherogenic lipoproteins in the bodily fluid of the subject.

In some aspects, the therapeutic device is a hemodialyzer. Some or all of the components comprising the hemodialyzer, such as a semi-permeable membrane, may be coated with MTMs.

In some aspects, the component of the therapeutic device is a filter, such as filer paper, e.g., cellulose, or a membrane filter, such as regenerated cellulose, cellulose acetate, nylon, PTFE, polypropylene, polyester, polyethersulfone, polyarylethersulfone, polycarbonate, and polyvinylpyrolidone. The filter may be coated with MTMs.

In some aspects, the therapeutic device can comprise a coating on any of one or more internal components of the therapeutic device that results in the immobilization of MTMs on the one or more components. For example, where the therapeutic device is an oxygenation device comprising a membrane, the MTMs can be coated on the membrane.

In some aspects, the component of a therapeutic device is a MTM-coated component of an oxygenation device. Examples of an oxygenation device are described in U.S. Application Pub. No. 2019/0167882, which is incorporated by reference in its entirety herein.

In some aspects, the component of a therapeutic device is a MTM-coated component of an ECMO device. Examples of an ECMO device are described in U.S. Application Pub. No. 2019/0167882, which is incorporated by reference in its entirety herein.

In some aspects, the component of a therapeutic device is a MTM-coated component of a dialysis device, for example a hemodialysis device or a peritoneal dialysis device. In the case of a dialysis device, the MTMs can be further used to detect, measure and remove MAMPs from a dialysis fluid.

In some aspects, the component of a therapeutic device is a MTM-coated component of an infusion device or a transfusion device. For example, the fluid can be sourced from a patient, a different patient or a fluid storage device, and the fluid deposit can be the patient. In some aspects, the infusion device can comprise a syringe comprising a filtering device, where a fluid from a subject such as blood can be drawn into the syringe, filtered, and returned to the subject, and where the syringe is coated with MTMs.

In some aspects, the component of a therapeutic device is a MTM-coated component of a drainage device. In these aspects, the fluid can be a bodily fluid, the fluid source can be a patient, and the fluid deposit can be the patient, a different patient or a fluid storage system. For example, a patient with hydrocephalus may require drainage of cerebrospinal fluid using a therapeutic device such as a percutaneous drainage device, and a filtration device can be coupled to the drainage line to filter the fluid. The fluid can then be analyzed for diagnostic applications, as described herein. In some aspects, the percutaneous drainage device can be used to drain a biliary, pleural or an abscess.

In some aspects, the component of a therapeutic device comprises a MTM-coated component of a pump, where the pump can comprise a negative or positive pressure pump depending on the system configuration. In some aspects, the fluid source is a higher pressure than the fluid deposit, and a pump is optional. For example, a fluid source can be the artery or vein of a patient, and the fluid deposit can be the vein of the patient.

### Filtration Devices

As suggested above, MTMs and compositions of the invention can be used in filtration devices (and related methods) to remove microbes and microbial components from a sample, such as a fluid.

The filtration devices of the present invention are limited only in that (i) they are devices (or components thereof) having filtration capacity, and (ii) they contain one or more MTMs of the invention. In a typical example, the filtration devices or at least a component thereof will be coated with MTMs or otherwise display MTMs on a surface of the device or component thereof.

The filtration devices of the invention include those comprising a paper filter, e.g., cellulose, or a membrane filter, such as regenerated cellulose, cellulose acetate, nylon, PTFE, polypropylene, polyester, polyethersulfone, polyarylethersulfone, polycarbonate, and polyvinylpyrolidone. The filter may be coated with MTMs. The filter may be further coated with a material such as Endexo® surface modifying macromolecules, which may decrease blood clot formation and provide additional PAMP- and bacteria-depleting functionality need for dialysis patients with PAMPEMIA and/or blood infections.

Alternatively, or in addition, the filtration devices of the invention comprise at least one component that is coated with MTMs of the invention, or otherwise display MTMs such that the MTMs are exposed to a sample, such as a biological sample, under conditions permitting binding of microbes or microbial components in the sample by the MTMs. Such components include, but are not limited to, supports (e.g. graphene), beads (e.g. gold particles), particles (including nanoparticles, microparticles, polymer microbeads, magnetic microbeads, and the like), fibers, screens, mesh, tubes, hollow fibers, scaffolds, plates, channels, magnetic materials, medical apparatuses (e.g., needles or catheters) or implants, dipsticks or test strips, filtration devices or membranes, cartridges (e.g. hollow fiber cartridges), microfluidic devices, mixing elements (e.g., spiral mixers), extracorporeal devices, and other substrates commonly utilized in therapeutic applications, and any combinations thereof. In some aspects, the therapeutic device or component thereof is a solid substrate, such as a filter or cartridge.

As an example, the filtration device may be a filtration cartridge comprising a substrate to which the MTMs of the invention are attached (see FIGS. 5 and 6). Substrates for use in such cartridges can be selected from a variety of known materials and formats. Examples include, but are not limited to, beads, nanoparticles, microparticles, polymer microbeads, magnetic microbeads, filters, fibers, screens, mesh, tubes, hollow fibers, scaffolds, plates, channels, gold particles, magnetic materials. Substrates within the cartridges as well as the other components of the filtration device may be made of any material, including, but not limited to, metal, metal alloy, polymer, plastic, paper, glass, fabric, packaging material, biological material such as cells, tissues, hydrogels, proteins, peptides, nucleic acids, and any combinations thereof. In some aspects, the substrate comprises filter paper, such as cellulose. In some aspects, the substrate comprises membrane filters, such as regenerated cellulose, cellulose acetate, nylon, PTFE, polypropylene, polyester, polyethersulfone, polyarylethersulfone, polycarbonate, and polyvinylpyrolidone.

The filtration devices of the invention may be used in a wide variety of applications including, but not limited to, methods of removing microbes or microbial components from a sample, such as a fluid. Such methods include contacting a bodily fluid of the subject with a filtration device of the invention under conditions that permit binding of microbes or microbial components by MTMs displayed by the filtration device, thus reducing the amount of microbes or microbial components in the bodily fluid of the subject. In one aspect, the microbial infection is a bacterial infection. In another aspect, the microbial infection is a viral infection. In further aspect, the microbial infection is a fungal infection. Such methods can be used to treat infectious diseases.

The filtration devices of the invention may also be used in non-biological applications. For example, the filtration devices of the invention may be used in a method that removes microbes or microbial components from an agricultural product, a food or beverage, an environmental sample, a pharmaceutical sample, etc.

### Systems and Methods for Filtering Fluids

The present invention is also directed to systems and methods for filtering fluids using the filtration devices, and optionally the therapeutic devices, of the invention. Such systems may be used in methods of removing microbes and microbial components from a fluid by binding target molecules of microbes, such as pathogens and components thereof (e.g. PAMPs), present in a fluid as the fluid flows through filtration device(s) of the system. The systems may optionally be used to also treat a fluid following through therapeutic device(s) of the system. The systems and methods can be used in diagnostic, therapeutic and filtration applications. As such, fluids such as blood or other fluids may be filtered to remove harmful pathogens and PAMPs and optionally treated (e.g., oxygenated, removal of carbon dioxide, combined with an agent such as a drug) before reintroduction of the fluid into a subject (e.g., a human or an animal patient) and/or to capture microbes for further analysis (e.g., detection, identification and antimicrobial susceptibility testing). Microbes and components thereof captured or filtered using systems and methods of the invention can include, for example, living or dead Gram-positive bacterial species, Gram-negative bacterial species, mycobacteria, fungi, parasites, viruses, or portions thereof. By combining these functions, a subject can receive a therapy, e.g., oxygenation of blood, while simultaneously removing pathogens and PAMPs from the blood.

Systems and methods of the invention can include obtaining a fluid (e.g., a bodily fluid) from a fluid source such as a patient. In certain aspects, the fluid, such as blood, may be obtained directly from a patient, for example, from an artery or a vein of a patient, and connected to the system for filtration and/or treatment. In some aspects, the fluid source may be a vial or other container in which a fluid may be stored.

After treatment, the filtered fluid may be deposited to a fluid deposit, for example, returned to the patient or a different patient, and/or the filtered fluid may be retained for further processing, for storage, e.g., a blood bank, or discarded. In certain aspects, blood or other fluids are siphoned directly from the patient or other fluid source and the fluid's natural pressure or flow (e.g. the patient's blood pressure) is used to force the fluid through the system. For example, the fluid source can be an artery of a patient, and the fluid deposit can be a vein of a patient, where the patient can be the same or a different patient.

In some aspects, a pump may be used to drive the fluid through the system. Extracorporeal blood pumps including roller pumps, pulsatile tube compression pumps, ventricular pumps, and centrifugal pumps are known in the art and, among others are contemplated for use with the invention.

Where a pump is used, the flow rate may be tunable to achieve the desired transit time of the target molecules in the fluid and the substrate-bound MTMs to change binding efficiency. Flow rates may be pre-set to provide an optimum transit time for filtering based on the fluid viscosity, substrate density, channel cross-section and other fluid-dynamic factors. Systems may include flow sensors and tunable pumps to allow for the automatic or manual monitoring of flow rates by computer or users. The tunable pumps allow the flow rate to be changed to a desired rate based on application requirements (e.g., to maximize binding). In some aspects, the flow rate may be fixed or tuned to be from about 50 mL/min to 3000 mL/min. Similarly, the flow rate may be tunable to the therapeutic device. In some aspects, the flow rate of the fluid through the therapeutic device can be the same or different than the flow rate of the fluid through the filtration device.

Systems and methods of the invention may include a heater or other means of temperature regulation to, for example, maintain a temperature that promotes target/MTM binding, maintain a temperature for fluid preservation, or to approximate body temperature before return of the filtered fluid to a patient. In some aspects, the fluid temperature may be maintained between 33-41 degrees Celsius.

FIG. 2 shows an exemplary filtration system 101. The system 101 includes one or more cartridges 103 comprising one or more MTMs of the invention. The system can include any number of filtration cartridges (i.e. a filtration device), where each filtration cartridge can comprise any number of substrates, where each substrate can comprise any number of MTMs. As an example, a system can include two filtration cartridges where the first filtration cartridge comprises one substrate comprising a first MTM and a second filtration cartridge comprises a second MTM, where the first and second MTMs can be the same or different. As another example, a system can include one filtration cartridge comprising multiple substrates, where each substrate comprises a different MTM. The system 101 can include an inlet/outlet 113 through which a fluid may be introduced to and/or removed from the system 101. In some aspects, the inlet and outlet can be separate components. The fluid can be transported between filtration cartridges 103 and within the system 101 generally through one or more channels 111. The fluid may be flowed through the channels 111 through the action of a pump 107. A temperature regulating device 115 such as a heater or a refrigerant may be included within the system 101 to maintain the fluid at a desired temperature. The system 101 may include one or more valves 105 to divert the flow of the fluid within the system 101. For example, a bypass 109 channel may be included in the system 101 along with a valve 105 such that activation of the valve diverts the fluid around one or more filtration cartridges 103 allowing the filtration cartridge 103 to be removed without stopping the flow of the fluid.

As discussed above, systems may include one or more inlets/outlets for adding and/or removing fluids from the system. The system may be a single-pass system where a fluid flows through the one or more filtration cartridges a single time before exiting the system for storage, re-introduction into a patient, analysis, or other post-filtration uses. In some aspects, the system may include a loop in order to pass fluid through the filtration cartridges two or more times to ensure maximum target binding and removal before removing the filtrate from the system.

As discussed above, filtration cartridges may be interchangeable within the system to allow for a full cartridge (where MTM microbe-binding domains are saturated) to be removed and a new cartridge (with free MTM microbe-binding domains) to be inserted. Valves and/or bypasses may be used to stop the flow of fluid through one or more filtration cartridges and optionally divert the flow of fluid around one or more filtration cartridges to permit filtration cartridges to be replaced. With the use of bypasses, the system can continue to run while filtration cartridges are removed and/or replaced.

As described above, detectable labels may be included in the MTMs that can provide a detectable signal upon the binding of a target molecule (e.g., microbe or microbial component) to the MTM. Filtration cartridges may include a transparent surface or window affording a view of the cartridge's interior while the system is in operation. The window should allow detection of the detectable label signal therethrough. For example, the window may be optically transparent where the detectable signal is an optical signal.

Through the window, the detectable label may be viewed such that the system, automatically, or a user, manually, may monitor target binding. The signal strength is indicative of the amount of binding activity and, therefore, given a finite amount of MTMs present in a given cartridge, is further indicative of the remaining filtering capacity of that cartridge. Accordingly, observation of that signal strength can inform a user or the system when a cartridge's filtration efficiency is low enough to warrant a cartridge change. If the signal reaches a certain threshold, as detected by a computer-linked sensor, or observed by a user, the computer or the user may stop the flow of fluid through that cartridge and remove it from the system to replace it with a fresh cartridge. Where sensors are used to detect the signal, the computer may notify the user (e.g., through a user interface) that the signal threshold has been reached and the cartridge should be changed. Alternatively, the computer may automatically divert flow to bypass the saturated cartridge. The computer may, through the use of computer-controlled valves, divert flow to fresh cartridges already linked to the system or may, through the use of computer-controlled motors, gantries, robotic arms, and the like, remove and replace the saturated cartridge automatically with a fresh cartridge.

Systems and methods of the invention may use other means to determine filtration cartridge saturation or reduced filtration efficiency. For example, the system may include a mechanism for weighing the cartridge and, optionally, an indicator to indicate the cartridge's weight to a user. Changes in the weight of the cartridge relative to a volume of fluid being filtered thereto can be indicative of the amount of bound target present in the cartridge and can therefore be used to determine when the MTMs are saturated, and the cartridge should be changed.

In some aspects, one of the components of the system, for example, the one or more substrates, undergoes a preprocessing step. For example, MTMs can be conjugated to a substrate and a cartridge can be filled with a buffer solution during storage and prior to use. The pH of the buffer solution can be maintained to optimally promote the stability of the MTMs. The buffer solution can be aqueous and can include one or more agents to act as a preservative and promote protein stability. Exemplary agents include but are not limited to a free-floating protein such as BSA, EDTA, glycerol, ethylene and glycol. The buffer solution can be flushed from the cartridge prior to connection with the subject to ensure that the buffer contents do not enter the subject's bloodstream. In some aspects, MTMs can be conjugated to a substrate and a cartridge can be lyophilized and stored in a dry format. Prior to use, the cartridge can be filled with a reconstitution buffer such as aqueous or distilled water for a certain amount of time (for example, less than one hour) and lightly agitated, for example, by turning the cartridge over several times. The reconstitution buffer can be flushed from the cartridge prior to connection with the subject to ensure that the buffer contents do not enter the subject's bloodstream.

In some aspects, the materials in contact with fluids are formed from inert, sterile, and biocompatible materials. In some aspects, components of the system may be coated with anticoagulants such as heparin warfarin, rivaroxaban, dabigatran, apixaban, edoxaban, enoxaparin, or fondaparinux to reduce the risk of thrombosis. In some aspects, the surface of the materials can be modified to reduce coagulation. Examples of coatings for surface modification include but are not limited to: Poly(ethylene oxide) (PEO) to increase the surface hydrophilicity; Albumin to reduce platelet adhesion; Pyrolytic carbon to reduce platelet adhesion and spreading on the surface; Phosphorylcholine surfaces that are predominantly lipid having a physiologically neutral pH on the outer surface of non-activated cell membranes to reduce protein and cell adhesion; Elastin-inspired polymer or synthesized elastin-inspired polymers to decreased fibrinogen adsorption and reduce proinflammatory cytokine release from monocytes; CTI to inhibit the activation of fXII and attenuate the deposition of protein; Immobilized heparin or heparin-mimicking molecules to activate antithrombin and attenuate the inflammatory response; a direct thrombin inhibitor grafted surface such as hirudin, bivalirudin, or argatroban to inhibit thrombin; Thrombomodulin or recombinant-thrombomodulin to promote the activation of protein C, thus limiting the coagulation by inactivating fVIIIa and fVa, important cofactors for fXa and thrombin generation; and Mannose-binding lectin (MBL) to decrease unintended platelet activation and coagulation in the blood contacting filters.

In some aspects, the cartridge substrates may be removable to permit further analysis of bound targets using methods described herein.

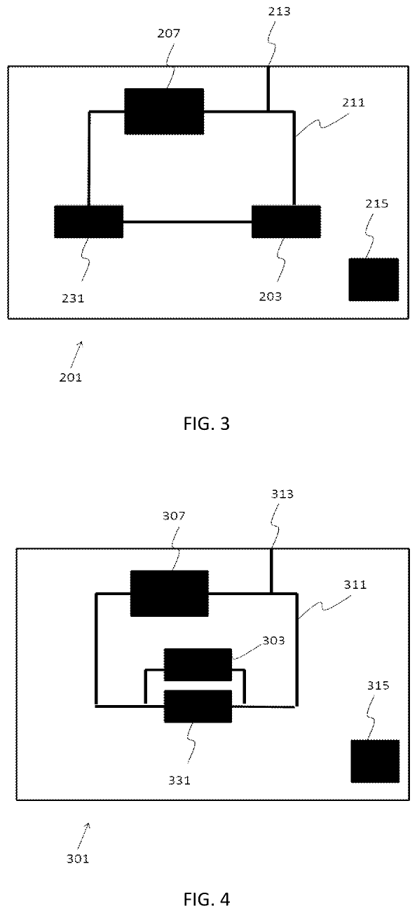

FIG. 3 shows an exemplary system 201. The system 201 includes a filtration device 203 comprising one or more substrate-bound MTMs with target-binding domains and one or more therapeutic devices 231. The system is configured in a flow pathway, shown as a circuit, where the filtration device 203 and therapeutic device 231 are in series. The system can be configured such that the fluid can flow through the filtration device 203 first or the therapeutic device 231 first.

The filtration device can be configured similar to filtration cartridge 103 of FIG. 2. The filtration device can comprise any number of substrates, where each substrate can comprise any number of MTMs. As an example, the filtration device can include two substrates where the first substrate comprises a first MTM and the second substrate comprises a second MTM where the first and second MTMs can be the same or different.

The one or more therapeutic devices 231 (FIG. 3) can comprise an oxygenation device, an extracorporeal device (e.g. an ECMO device), a blood pump device, a heart-lung device, a dialysis device, a drainage device, a blood transfusion device, an infusion device, a temperature management device, a pressure management device, a plasma separator, a hemoperfusion cartridge, an adsorbent device, a monitoring device, a cytokine reduction system, a pathogen reduction system, a PAMP reduction system, a respiration device, a ventilation devices, and a catheter or tube used in a medical procedure.

The system 201 can include an inlet/outlet 213 through which a fluid may be introduced to and/or removed from the system 201. In some aspects, the inlet and outlet can be separate components. The fluid can be transported between filtration device 203 and therapeutic device 231 and within the system 201 generally through one or more channels 211. In some aspects, the fluid may be flowed through the channels 211 through the action of a pump 207. In some aspects, a pump is optional where the fluid source is at a higher pressure than the fluid deposit, for example, where the fluid source is an artery or vein of a subject, and the fluid deposit is a vein of the subject. A temperature regulating device 215 such as a heater or a refrigerant may be included within the system 201 to maintain the fluid at a desired temperature. The system 201 may include a bypass channel and one or more valves (not shown) to divert the flow of the fluid within the system 201. For example, a bypass channel may be included in the system 201 along with a valve such that activation of the valve diverts the fluid around the filtration device 203 or the therapeutic device 231, for example to allow the filtration device 203 to be removed without stopping the flow of the fluid.

The system 201 may include one or more inlets/outlets for adding and/or removing fluids from the system. The system may be a single-pass system where a fluid flows through the one or more devices 203 or 231 a single time before exiting the system for storage, re-introduction into a patient, analysis, or other post-filtration uses. In some aspects, the system may include a loop in order to pass fluid through one or both of the devices 203 and 231 two or more times to ensure maximum target binding and removal before removing the filtrate from the system and desired effect from the therapeutic device 231.

The filtration device 203 may be interchangeable within the system to allow for a full device (where MTM binding domains are saturated) to be removed and a new device (with free MTM binding domains) to be inserted. Valves and/or bypasses may be used to stop the flow of fluid through the filtration device 203 and optionally divert the flow of fluid around the filtration device 203 to permit the device or a device component to be replaced. With the use of bypasses, the system can continue to run while the device or one of its components are removed and/or replaced.

FIG. 4 shows an exemplary system 301. The system 301 includes a filtration device 303 comprising one or more substrate-bound MTMs with target-binding domains and one or more therapeutic devices 331. The system is configured as a flow pathway, shown as a circuit, similar to system 201 of FIG. 3, however the filtration device 303 and therapeutic device 331 are in parallel.

The system 301 can include an inlet/outlet 313 through which a fluid may be introduced to and/or removed from the system 301. In some aspects, the inlet and outlet can be separate components. The fluid can be transported between filtration device 303 and therapeutic device 331 and within the system 301 generally through one or more channels 311. In some aspects, the fluid may be flowed through the channels 311 through the action of a pump 307. In some aspects, a pump is optional where the fluid source is at a higher pressure than the fluid deposit, for example, where the fluid source is an artery or vein of a subject, and the fluid deposit is a vein of the subject. A temperature regulating device 315 such as a heater or a refrigerant may be included within the system 301 to maintain the fluid at a desired temperature. The system 301 may include a bypass channel and one or more valves (not shown) to divert the flow of the fluid within the system 301. For example, a bypass channel may be included in the system 301 along with a valve such that activation of the valve diverts the fluid around the filtration device 303 or the therapeutic device 331, for example to allow the filtration device 303 to be removed without stopping the flow of the fluid.

The system 301 may include one or more inlets/outlets for adding and/or removing fluids from the system. The system may be a single-pass system where a fluid flows through the one or more devices 303 or 331 a single time before exiting the system for storage, re-introduction into a patient, analysis, or other post-filtration uses. In some aspects, the system may include a loop in order to pass fluid through one or both of the devices 303 and 331 two or more times to ensure maximum target binding and removal before removing the filtrate from the system and desired effect from the therapeutic device 331.

The filtration device 303 may be interchangeable within the system to allow for a full device (where MTM binding domains are saturated) to be removed and a new device (with free MTM binding domains) to be inserted. Valves and/or bypasses may be used to stop the flow of fluid through the filtration device 303 and optionally divert the flow of fluid around the filtration device 303 to permit the device or a device component to be replaced. With the use of bypasses, the system can continue to run while the device or one of its components are removed and/or replaced.

In some aspects, the therapeutic device comprises the filtration device. In some aspects, the filtration device can comprise a coating on any of one or more internal components of the therapeutic device. For example, where the therapeutic device is an oxygenation device comprising a membrane, the MTM can be coated on the membrane. In some aspects, the therapeutic device can comprise one or more substrate-bound MTMs with target-binding domains.

In some aspects, the therapeutic device can comprise an oxygenation device. Examples of an oxygenation device are described in U.S. Patent Application Pub. No. 20190167882, which is incorporated by reference in its entirety herein.

In some aspects, the therapeutic device can comprise an ECMO device. Examples of an ECMO device are described in U.S. Patent Application Pub. No. 20190167882, which is incorporated by reference in its entirety herein.

In some aspects, the therapeutic device can comprise a dialysis device, for example a hemodialysis device or a peritoneal dialysis device. In the case of a dialysis device, the MTMs can be further used to detect, measure and remove MAMPs from a dialysis fluid.

In some aspects, the therapeutic device can comprise an infusion device or a transfusion device. For example, the fluid can be a fluid (mixed with an agent such as a drug), the fluid source can be a patient, a different patient or a fluid storage device, and the fluid deposit can be the patient. In some aspects, the infusion device can comprise a syringe comprising a filtering device, where a fluid from a subject such as blood can be drawn into the syringe, filtered, and returned to the subject.

In some aspects, the therapeutic device can comprise a drainage device. In these aspects, the fluid can be a bodily fluid, the fluid source can be a patient, and the fluid deposit can be the patient, a different patient or a fluid storage system. For example, a patient with hydrocephalus may require drainage of cerebrospinal fluid using a therapeutic device such as a percutaneous drainage device, and a filtration device can be coupled to the drainage line to filter the fluid. The fluid can then be analyzed for diagnostic applications, as described herein. In some aspects, the percutaneous drainage device can be used to drain a biliary, pleural or an abscess.

In any of these aspects, the system can be configured for use in a clinical setting or an in-home setting.

In some aspects, the system comprises a pump (as described in FIGS. 2-4), where the pump can comprise a negative or positive pressure pump depending on the system configuration. In some aspects, the fluid source is a higher pressure than the fluid deposit, and a pump is optional. For example, a fluid source can be the artery or vein of a patient, and the fluid deposit can be the vein of the patient.

As one skilled in the art would recognize as necessary or best-suited for the systems and methods of the invention, devices and methods of the invention may include one or more servers and/or computing devices that may include one or more of processor (e.g., a central processing unit (CPU), a graphics processing unit (GPU), etc.), computer-readable storage device (e.g., main memory, static memory, etc.), or combinations thereof which communicate with each other via a bus. Computer systems may be operable to interpret data received from weight or imaging sensors to determine when a cartridge is saturated or has become inefficient and should be changed. Computer systems may also be operable to divert fluid flow through the control of various valves within the filtration system.

A processor may include any suitable processor known in the art, such as the processor sold under the trademark XEON E7 by Intel (Santa Clara, CA) or the processor sold under the trademark OPTERON 6200 by AMD (Sunnyvale, CA).

Memory preferably includes at least one tangible, non-transitory medium capable of storing: one or more sets of instructions executable to cause the system to perform functions described herein (e.g., software embodying any methodology or function found herein); data; or both. While the computer-readable storage device can, in an exemplary embodiment, be a single medium, the term “computer-readable storage device” should be taken to include a single medium or multiple media (e.g., a centralized or distributed database, and/or associated caches and servers) that store the instructions or data. The term “computer-readable storage device” shall accordingly be taken to include, without limit, solid-state memories (e.g., subscriber identity module (SIM) card, secure digital card (SD card), micro SD card, or solid-state drive (SSD)), optical and magnetic media, hard drives, disk drives, and any other tangible storage media.

Any suitable services can be used for storage such as, for example, Amazon Web Services, memory of server, cloud storage, another server, or other computer-readable storage. Cloud storage may refer to a data storage scheme wherein data is stored in logical pools and the physical storage may span across multiple servers and multiple locations. Storage may be owned and managed by a hosting company. Preferably, storage is used to store records as needed to perform and support operations described herein.

Input/output devices according to the invention may include one or more of a video display unit (e.g., a liquid crystal display (LCD) or a cathode ray tube (CRT) monitor), an alphanumeric input device (e.g., a keyboard), a cursor control device (e.g., a mouse or trackpad), a disk drive unit, a signal generation device (e.g., a speaker), a touchscreen, a button, an accelerometer, a microphone, a cellular radio frequency antenna, a network interface device, which can be, for example, a network interface card (NIC), Wi-Fi card, or cellular modem, or any combination thereof. Input/output devices such as user interfaces may be used to provide information regarding filtration status to a user and to receive input to direct the operation of temperature regulators, pumps, valves, and other components within the system when not operated automatically by the computing system.

One skilled in the art will recognize that any suitable development environment or programming language may be employed to allow the operability described herein for various systems and methods of the invention. For example, systems and methods herein can be implemented using Perl, Python, C++, C #, Java, JavaScript, Visual Basic, Ruby on Rails, Groovy and Grails, or any other suitable tool.

The present invention provides a method for filtering a fluid comprising providing a fluid from a fluid source to a filtration device of the invention, filtering the fluid in the device, and providing the fluid to a fluid deposit. In some aspects, filtering the fluid in the filtration device removed microbes and/or microbial components. The method can optionally include analyzing the fluid, where analyzing the fluid comprises detecting and/or identifying one or more microbes or microbe components present in the fluid, for example using a mass spectrometric analysis method.

The present invention provides a method for filtering and/or treating a fluid comprising providing a fluid from a fluid source to a therapeutic device of the invention, filtering and/or treating the fluid in the device, and providing the fluid to a fluid deposit. In some aspects, treating the fluid in the therapeutic device can include providing a therapy to the fluid using any of the therapeutic devices described herein and/or filtering the fluid using any of the therapeutic devices described herein. In some aspects, providing a therapy to a fluid comprises oxygenating the fluid and/or removing carbon dioxide from the fluid. In some aspects, providing a therapy to a fluid comprises adding an agent such as a drug to the fluid and/or infusing the fluid to a subject. In some aspects, providing a therapy to a fluid comprises removing, e.g., draining, the fluid from a subject. The method can optionally include analyzing the fluid, where analyzing the fluid comprises detecting and/or identifying one or more microbes or microbe components present in the fluid, for example using a mass spectrometric analysis method.

The present invention provides a method for treating a subject such as a human or an animal comprising removing a fluid from a fluid source, providing the fluid to a therapeutic device, treating the fluid in the device, and providing the fluid to a fluid deposit. In some aspects, treating the fluid in the therapeutic device can include providing a therapy to the fluid using any of the therapeutic devices described herein and/or filtering the fluid using any of the therapeutic device described herein. In some aspects, providing a therapy to a fluid comprises oxygenating the fluid and/or removing carbon dioxide from the fluid. In some aspects, providing a therapy to a fluid comprises adding an agent such as a drug to the fluid and/or infusing the fluid to a subject. In some aspects, providing a therapy to a fluid comprises removing, e.g., draining, the fluid from a subject. The method can optionally include analyzing the fluid, where analyzing the fluid comprises detecting and/or identifying one or more microbes or microbe components present in the fluid, for example using a mass spectrometric analysis method. An example includes treating a patient who is in need of oxygenated blood and is septic, where the method includes connecting a patient to a system where the system comprises a veno-venous oxygenation or ECMO device and a therapeutic device according to any of the configurations described herein. The patient is connected to the system via cannulation where venous blood is siphoned into the oxygenation or ECMO device and the therapeutic device. Carbon dioxide is removed from the blood and/or blood is oxygenated and filtered and returned to the systemic venous circulation of the patient. Optionally, a sample can be taken from the therapeutic device to identify the captured microbes or for antimicrobial susceptibility testing. Another exemplary method includes connecting a patient to a system where the system comprises an arterio-venous oxygenation or ECMO device and a therapeutic device according to any of the configurations described herein. The patient is connected to the system via cannulation where arterial blood is siphoned into the oxygenation or ECMO device and the therapeutic device. Carbon dioxide is removed from the blood and/or blood is oxygenated and filtered and returned to the systemic venous circulation of the patient. Optionally, a sample can be taken from the therapeutic device to identify the captured microbes or for antimicrobial susceptibility testing.

Devices, systems and methods of the invention may be used to prepare and analyze a variety of biological fluids. In some aspects, microbes are filtered from the fluid and may be subsequently analyzed and/or identified.

As described above, biological fluids can include a bodily fluid and may be collected in any clinically acceptable manner. Fluids can include, but are not limited to, mucous, phlegm, saliva, sputum, blood, plasma, serum, serum derivatives, bile, sweat, amniotic fluid, menstrual fluid, mammary fluid, peritoneal fluid, interstitial fluid, urine, semen, synovial fluid, interocular fluid, a joint fluid, an articular fluid, and cerebrospinal fluid (CSF). A fluid may also be a fine needle aspirate or biopsied tissue. Blood fluids can be obtained by standard phlebotomy procedures and may be separated into components such as plasma for analysis. Centrifugation can be used to separate out fluid components to obtain plasma, buffy coat, erythrocytes, cells, pathogens and other components.

The filtration and therapeutic devices, and systems of the invention using the devices, can be used in transplant applications such as in the transplant of T cells, cartilage, stem cells or in cell culture media. As an example, the system can be used to remove viruses, mycoplasma, bacteria, fungi before T cells are separated from a donor's blood. In some cases, the system can be used to remove viruses, mycoplasma, bacteria, fungi during the process of separating T cells from a donor's blood. In some cases, the system can be used to remove viruses, mycoplasma, bacteria, fungi after separating T cells from a donor's blood.

Systems comprising the filtration devices of the invention may also be used to filter environmental fluids including, for example, saturated soil water, groundwater, surface water, unsaturated soil water; and fluids from industrialized processes such as waste water. Agricultural fluids that can be filtered using the systems of the invention include, for example, crop fluids, such as grain and forage products, such as soybeans, wheat, and corn.

After filtration, the cartridge contents may be subjected to additional analysis to, for example, identify the captured microbes or for antimicrobial susceptibility testing, as discussed herein. Isolation can include elution of the microbe to release them from the bound substrate for further analysis.

As another non-limiting example, the devices and systems of the invention can be used to inhibit or prevent one or more pathogens from entering a subject, e.g., into the eye(s), nose, mouth, and/or respiratory system, including the airway, lungs and blood vessels, and blood; to decrease the pathogen load on the subject; and/or to treat a subject having one or more infections.

The systems and methods of the invention can also be used to detect, diagnose (e.g., detect and/or identify) and/or treat a subject, such as a human, having a microbial infection. Thus, each of the relevant procedures that take place, from first detecting a pathogen in a sample, to the treatment and removal of the pathogen from a bodily fluid of a subject, can be practiced using the systems and methods of the invention. In one example non-limiting, the invention includes a method of (i) detecting a pathogen in a sample from a subject using a rapid detection device, (ii) diagnosing the pathogen in the sample using a diagnostic device, and (iii) treating the patient using a filtration device to remove the pathogen from the subject. Additional steps can be included in the method. For example, in another example, the invention includes a method of (i) detecting a pathogen in a sample from a subject using a rapid detection device, (ii) diagnosing the pathogen in the sample using a diagnostic device, (iii) identifying the pathogen in the sample using an identification means, and (iv) treating the patient using a filtration device. The method can be practiced by obtaining additional samples from the subject, for example, after treatment, as a means for monitoring disease progression in the subject. Thus, and in another example, the invention includes a method of (i) detecting a pathogen in a sample from a subject using a rapid detection device, (ii) diagnosing the pathogen in the sample using a diagnostic device, (iii) identifying the pathogen in the sample using an identification means, (iv) optionally detecting the pathogen in a second sample from the same subject using a rapid detection device, (v) treating the patient using a filtration device, and (vi) optionally detecting the pathogen in a third sample from the same subject using a rapid detection device.

The rapid detection devices of the invention include lateral flow assays, wherein at least one component of the lateral flow assay comprises one or more MTMs. In some aspects, the rapid detection device can be used to recognize an active infection and assess infection severity, including sepsis, in a short period of time, for example, within minutes of receiving a sample.

The identification means of the invention include devices and assays to detect and/or identify a microbe or microbial component in a sample as described herein. Such means include: volatile organic compound methods, spectrometry (e.g., Raman spectroscopy; FFT (Fast-Fourier Transform); Fourier-Transform Infrared Spectroscopy (FTIR); infrared spectrometry; Nuclear Magnetic Resonance (NMR) spectrometry), electrochemical detection, polynucleotide detection, fluorescence anisotropy, fluorescence resonance energy transfer, electron transfer, enzyme assay, magnetism, electrical conductivity, electrochemical detection, isoelectric focusing, lateral flow assay (LFA), microfluidics, amino acid sequencing, nucleic acid sequencing, flow cytometry, chromatography, immunoprecipitation, immunoseparation, aptamer binding, filtration, electrophoresis, use of a CCD camera, immunoassay, ELISA, Gram staining, immunostaining, microscopy, immunofluorescence, size/weight/charge detection, CRISPR, western blot, polymerase chain reaction (PCR), RT-PCR, isothermal amplification, sequencing, next gen sequencing, fluorescence in situ hybridization, mass spectrometry, SPR, and LSPR.

The invention includes systems for conducting such methods. An exemplary system comprises a rapid detection device, a diagnostic device, and a filtration device, and optionally an identification means.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further aspects thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various aspects and equivalents thereof.

What is claimed is:

1. A collectin-based engineered microbe-targeting molecule (MTM) comprising the amino acid sequence of any one of SEQ ID NOs:6, 7, 8, and 9 or a sequence variant thereof having at least 85% sequence identity to any one of SEQ ID NOs:6, 7, 8, and 9.

2. The collectin-based engineered MTM according to claim 1 comprising the amino acid sequence of SEQ ID NO:6 or a sequence variant thereof having at least 85% sequence identity to SEQ ID NO:6.

3. The collectin-based engineered MTM according to claim 1 comprising the amino acid sequence of SEQ ID NO:7 or a sequence variant thereof having at least 85% sequence identity to SEQ ID NO:7.

4. The collectin-based engineered MTM according to claim 1 comprising the amino acid sequence of SEQ ID NO:8 or a sequence variant thereof having at least 85% sequence identity to SEQ ID NO:8.

5. The collectin-based engineered MTM according to claim 1 comprising the amino acid sequence of SEQ ID NO:9 or a sequence variant thereof having at least 85% sequence identity to SEQ ID NO:9.

6. A polynucleotide sequence encoding a collectin-based engineered MTM of any one of claims 1-5, or a complementary strand thereof.

7. A cloning or expression vector comprising a polynucleotide sequence of claim 6.

8. A cell comprising a polynucleotide sequence of claim 6.

9. A cell comprising a cloning or expression vector of claim 7.

10. A method of producing a collectin-based engineered MTM comprising culturing a cell of claim 8 under conditions promoting expression of the collectin-based engineered MTM, and recovering the collectin-based engineered MTM from the cell or cell culture.

11. A method of producing a collectin-based engineered MTM comprising culturing a cell of claim 9 under conditions promoting expression of the collectin-based engineered MTM, and recovering the collectin-based engineered MTM from the cell or cell culture.

12. A composition comprising one or more engineered MTMs, wherein the one or more engineered MTMs is selected from any of the engineered MTMs of claims 1-5.

13. The composition of claim 12, further comprising at least one naturally-occurring MTM.

14. The composition of claim 12, further comprising one or more antimicrobial agents.

15. The composition of claim 13, further comprising one or more antimicrobial agents.

16. A system comprising:
at least one filtration device comprising one or more collectin-based engineered MTM of any one of claims 1-5,
wherein the system is configured to receive a fluid from a fluid source and to return the fluid to a fluid deposit.

17. The system of claim 16, wherein the fluid source is a subject or a fluid storage system.

18. The system of claim 16, wherein the fluid deposit is a subject that is the fluid source, a subject that is not the fluid source, or a filtered fluid storage system.

19. The system of claim 16, wherein the fluid is blood and the fluid source is the artery of a subject and the fluid deposit is a vein of the subject, or wherein the fluid is blood and the fluid source is the vein of a subject and the fluid deposit is the vein of the subject.

20. The system of claim 16, further comprising at least one therapeutic device.

21. The system of claim 20, wherein the at least one therapeutic device comprises an oxygenation device, an ECMO device, a blood pump device, a heart-lung device, a dialysis device, a drainage device, a blood transfusion device, an infusion device, a temperature management device, a pressure management device, a filtration device, a plasma separator, a hemoperfusion cartridge, an adsorbent device, a monitoring device, a cytokine reduction system, a pathogen reduction system, a PAMP reduction system, a respiration device, a ventilation device, or a catheter or tube used in a medical procedure.

22. The system of claim 20, wherein at least one therapeutic device comprises a filtration device.

23. A method for filtering a fluid comprising:
providing a fluid from a fluid source to the system of claim 16;
filtering the fluid of one or more microbes or microbe in the system; and
providing the filtered fluid to a fluid deposit.

24. The method of claim 23, further comprising providing a therapy to the fluid using at least one therapeutic device.

25. The method of claim 24, wherein providing a therapy to the fluid comprises one or more of oxygenating the fluid, removing carbon dioxide from the fluid, adding an agent such as a drug to the fluid and infusing the fluid to a subject, or removing the fluid from a subject.

26. A method for treating a subject comprising:
removing a fluid from a fluid source;
providing the fluid to the system of claim 16;
treating the fluid in the system; and
providing the fluid to a fluid deposit.

27. The method of claim 26, wherein treating the fluid in the system comprises filtering the fluid of one or more microbes or microbe components using a filtration device.

28. The method of claim 27, wherein treating the fluid in the system further comprises providing a therapy to the fluid using at least one therapeutic device.

29. The method of claim 28, wherein providing a therapy to a fluid comprises one or more of oxygenating the fluid, removing carbon dioxide from the fluid, adding an agent such as a drug to the fluid and infusing the fluid to a subject, or removing the fluid from a subject.

30. A diagnostic device comprising at least one component coated with, or otherwise displaying, one or more collectin-based engineered MTM of any one of claims 1-5.

31. A method of detecting a microbe in a sample, comprising contacting a sample suspected of containing a microbe with a diagnostic device of claim 30 under conditions permitting binding of a microbe or a component of a microbe by MTMs displayed by the at least one component of the diagnostic device, thereby detecting a microbe in a sample.

32. A method of detecting a microbial infection in a subject, comprising contacting a biological sample of a subject suspected of having a microbial infection with a diagnostic device of claim 30 under conditions permitting binding of microbes or microbial components by MTMs displayed by the at least one component of the diagnostic device, thereby detecting a microbial infection in the subject.

33. A method of diagnosing a microbial infection in a subject, comprising contacting a biological sample of a subject suspected of having a microbial infection with a diagnostic device of claim 30 under conditions permitting binding of microbes or microbial components by MTMs displayed by the at least one component of the diagnostic device, thereby diagnosing a microbial infection in the subject.

34. A method of preparing a sample for detecting a microbial infection in a subject, comprising isolating from a sample a microbe or microbial component bound to the MTMs on the diagnostic device of claim 30, and digesting the isolated microbe or microbial component with an antimicrobial mixture.

35. A therapeutic device comprising at least one component coated with, or otherwise displaying, one or more collectin-based engineered MTM of any one of claims 1-5.

36. A method of treating a microbial infection in a subject, comprising contacting a bodily fluid of a subject having a microbial infection with a therapeutic device of claim 35 under conditions permitting binding of microbes by MTMs displayed by the at least one component of the therapeutic device, thereby treating a microbial infection in the subject.

37. A method of reducing microbes or microbial components in a bodily fluid of a subject, comprising contacting a bodily fluid of a subject with a therapeutic device of claim 35 under conditions permitting binding of microbes or microbial components by MTMs displayed by the at least one component of the therapeutic device, thereby reducing microbes or microbial components in a bodily fluid of the subject.

38. A filtration device comprising at least one component coated with, or otherwise displaying, one or more collectin-based engineered MTM of any one of claims 1-5.

39. A method of filtering a microbe or microbial component from a fluid, comprising contacting a fluid containing a microbe or microbial component with a filtration device of claim 38 under conditions permitting binding of a microbe or microbial component by MTMs displayed by the at least one component of the filtration device, thereby filtering a microbe or microbial component from a fluid.

40. A rapid detection device comprising a lateral flow assay, wherein at least one component of the lateral flow assay comprises one or more collectin-based engineered MTM of any one of claims 1-5.

41. A system comprising the diagnostic device of claim 30, the filtration device of claim 38, the rapid detection device of claim 40, and optionally an identification means, wherein the identification means utilizing any one of the following: volatile organic compound methods, spectrometry (e.g., Raman spectroscopy; FFT (Fast-Fourier Transform); Fourier-Transform Infrared Spectroscopy (FTIR); infrared spectrometry; Nuclear Magnetic Resonance (NMR) spectrometry), electrochemical detection, polynucleotide detection, fluorescence anisotropy, fluorescence resonance energy transfer, electron transfer, enzyme assay, magnetism, electrical conductivity, electrochemical detection, isoelectric focusing, lateral flow assay (LFA), microfluidics, amino acid sequencing, nucleic acid sequencing, flow cytometry, chromatography, immunoprecipitation, immunoseparation, aptamer binding, filtration, electrophoresis, use of a CCD camera, immunoassay, ELISA, Gram staining, immunostaining, microscopy, immunofluorescence, size/weight/charge detection, CRISPR, western blot, polymerase chain reaction (PCR), RT-PCR, isothermal amplification, sequencing, next gen sequencing, fluorescence in situ hybridization, mass spectrometry, SPR, and LSPR.

42. A method comprising,
(a) detecting a pathogen in a first sample from a subject using the rapid detection device of claim 40,
(b) diagnosing the pathogen in the first sample using a diagnostic device of claim 30,
(c) optionally identifying the pathogen in the first sample using an identification means,
(d) optionally detecting the pathogen in a second sample from the same subject using the rapid detection device of claim 40,
(e) treating the subject using the filtration device of claim 38, and
(f) optionally detecting the pathogen in a third sample from the same subject using the rapid detection device of claim 40,
wherein the identification means utilizing any one of the following: volatile organic compound methods, spectrometry (e.g., Raman spectroscopy; FFT (Fast-Fourier Transform); Fourier-Transform Infrared Spectroscopy (FTIR); infrared spectrometry; Nuclear Magnetic Resonance (NMR) spectrometry), electrochemical detection, polynucleotide detection, fluorescence anisotropy, fluorescence resonance energy transfer, electron transfer, enzyme assay, magnetism, electrical conductivity, electrochemical detection, isoelectric focusing, lateral flow assay (LFA), microfluidics, amino acid sequencing, nucleic acid sequencing, flow cytometry, chromatography, immunoprecipitation, immunoseparation, aptamer binding, filtration, electrophoresis, use of a CCD camera, immunoassay, ELISA, Gram staining, immunostaining, microscopy, immunofluorescence, size/weight/charge detection, CRISPR, western blot, polymerase chain reaction (PCR), RT-PCR, isothermal amplification, sequencing, next gen sequencing, fluorescence in situ hybridization, mass spectrometry, SPR, and LSPR.


SEQUENCE LISTING (20 sequences)

SEQ ID NO: 1 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 2 - protein, 228 aa, Homo sapiens
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 3 - protein, 141 aa, Homo sapiens
AASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 4 - protein, 115 aa, Homo sapiens
VGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 5 - protein, 148 aa, Homo sapiens
PDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 6 - protein, 381 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEINITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 7 - protein, 383 aa, Artificial Sequence
  Recombinantly-produced fusion protein
AKTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 8 - protein, 352 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATSKQVGNKFFLTNGEEVITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 9 - protein, 383 aa, Artificial Sequence
  Recombinantly-produced fusion protein
AKTEPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 10 - protein, 10 aa, Homo sapiens
NEGEPNNAGS

SEQ ID NO: 11 - protein, 15 aa, Homo sapiens
LLLKNGQWNDVPCST

SEQ ID NO: 12 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 13 - protein, 278 aa, Homo sapiens [sequence omitted]

SEQ ID NO: 14 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGALGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 15 - protein, 277 aa, Homo sapiens
MNGFASLLRRNQFILLVLFLLQIQSLGLDIDSRPTAEVCATHTISPGPKGDDGEKGDPGEEGKHGKVGRMGPKGIKGELGDMGDQGNIGKTGPIGKKGDKGEKGLLGIPGEKGKAGTVCDCGRYRKFVGQLDISIARLKTSMKFVKNVIAGIRETEEKFYYIVQEEKNYRESLTHCRIRGGMLAMPKDEAANTLIADYVAKSGFFRVFIGVNDLEREGQYMFTDNTPLQNYSNWNEGEPSDPYGHEDCVEMLSSGRWNDTECHLTMYFVCEFIKKKK

SEQ ID NO: 16 - protein, 742 aa, Homo sapiens
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGPSGDPGPPGPPGKEGLPGPQGPPGFQGLQGTVGEPGVPGPRGLPGLPGVPGMPGPKGPPGPPGPSGAVVPLALQNEPTPAPEDNGCPPHWKNFTDKCYYFSVEKEIFEDAKLFCEDKSSHLVFINTREEQQWIKKQMVGRESHWIGLTDSERENEWKWLDGTSPDYKNWKAGQPDNWGHGHGPGEDCAGLIYAGQWNDFQCEDVNNFICEKDRETVLSSAL

SEQ ID NO: 17 - protein, 321 aa, Bos taurus
MLPLPLSILLLLTQSQSFLGEEMDVYSEKTLTDPCTVVVCAPPADSLRGHDGRDGKEGPQGEKGDPGPPGMPGPAGREGPSGRQGSMGPPGTPGPKGEPGPEGGVGAPGMPGSPGPTGLKGERGTPGPGGAIGPQGPSGAMGPPGLKGDRGDPGEKGAKGETSVLEVDTLRQRMRNLEGEVQRLQNIVTQYRKAVLFPDGQAVGEKIFKTAGAVKSYSDAEQLCREAKGQLASPRSSAENEAVTQLVRAKNKHAYLSMNDISKEGKFTYPTGGSLDYSNWAPGEPNNRAKDEGPENCLEIYSDGNWNDIECREERLVICEF

SEQ ID NO: 18 - protein, 371 aa, Bos taurus
MLLLPLSVLLLLTQPWRSLGAEMKIYSQKTLANGCTLVVCRPPEGGLPGRDGQDGREGPQGEKGDPGSPGPAGRAGRPGPAGPIGPKGDNGSAGEPGPKGDTGPPGPPGMPGPAGREGPSGKQGSMGPPGTPGPKGDTGPKGGMGAPGMQGSPGPAGLKGERGAPGELGAPGSAGVAGPAGAIGPQGPSGARGPPGLKGDRGDPGERGAKGESGLADVNALKQRVTILEGQLQRLQNAFSRYKKAVLFPDGQAVGKKIFKTAGAVKSYSDAQQLCREAKGQLASPRSAAENEAVAQLVRAKNNDAFLSMNDISTEGKFTYPTGESLVYSNWASGEPNNNNAGQPENCVQIYREGKWNDVPCSEPLLVICEF

SEQ ID NO: 19 - protein, 271 aa, Homo sapiens
MRGNLALVGVLISLAFLSLLPSGHPQPAGDDACSVQILVPGLKGDAGEKGDKGAPGRPGRVGPTGEKGDMGDKGQKGSVGRHGKIGPIGSKGEKGDSGDIGPPGPNGEPGLPCECSQLRKAIGEMDNQVSQLTSELKFIKNAVAGVRETESKIYLLVKEEKRYADAQLSCQGRGGTLSMPKDEAANGLMAAYLAQAGLARVFIGINDLEKEGAFVYSDHSPMRTFNKWRSGEPNNAYDEEDCVEMVASGGWNDVACHTTMYFMCEFDKENM

SEQ ID NO: 20 - protein, 371 aa, Bos taurus
MLLLPLSVLLLLTQPWRSLGAEMTTFSQKILANACTLVMCSPLESGLPGHDGQDGRECPHGEKGDPGSPGPAGRAGRPGWVGPIGPKGDNGFVGEPGPKGDTGPRGPPGMPGPAGREGPSGKQGSMGPPGTPGPKGETGPKGGVGAPGIQGFPGPSGLKGEKGAPGETGAPGRAGVTGPSGAIGPQGPSGARGPPGLKGDRGDPGETGAKGESGLAEVNALKQRVTILDGHLRRFQNAFSQYKKAVLFPDGQAVGEKIFKTAGAVKSYSDAEQLCREAKGQLASPRSSAENEAVTQMVRAQEKNAYLSMNDISTEGRFTYPTGEILVYSNWADGEPNNSDEGQPENCVEIFPDGKWNDVPCSKQLLVICEF